(19) United States
(12) Patent Application Publication
(10) Pub. No.: US 2012/0076781 A1
(43) Pub. Date: Mar. 29, 2012

(54) METHODS OF TREATING CANCERS WITH HER3 ANTISENSE OLIGONUCLEOTIDES

(75) Inventors: Yixian Zhang, Piscataway, NJ (US); Zhengxing Qu, Warren, NJ (US); Lee Martin Greenberger, Montclair, NJ (US)

(73) Assignees: SANTARIS PHARMA A/S, Hoersholm (DK); ENZON PHARMACEUTICALS, INC., Bridgewater, NJ (US)

(21) Appl. No.: 13/264,265

(22) PCT Filed: Apr. 14, 2010
(86) PCT No.: PCT/US10/31005

§ 371 Date: Dec. 14, 2011

Related U.S. Application Data
(60) Provisional application No. 61/169,093, filed on Apr. 14, 2009.

(51) Int. Cl. A61K 31/713; A61K 39/395; A61P 35/02; A61P 35/00

(52) U.S. Cl. 424/133.1; 514/44 A

(57) ABSTRACT

One aspect of the invention provides methods for treating cancers which are resistant to treatment with a protein tyrosine kinase inhibitor by co-treatment with the protein tyrosine kinase inhibitor and one or more antisense oligomers that reduce the expression of HER3 and/or HER2 and/or EGFR. Another aspect of the invention provides methods for treating cancers by co-treatment with an inhibitor of HER2 and one or more antisense oligomers that reduce the expression of HER3.

### 1. CROSS-REFERENCE TO RELATED APPLICATIONS

This application claims priority to U.S. Provisional Patent Application Ser. No. 61/169,093 filed Apr. 14, 2009, which is hereby incorporated by reference in its entirety.

### 2. BACKGROUND

HER3 is a member of the ErbB family of receptor tyrosine kinases, which includes four different receptors: ErbB-1 (EGFR, HER1), ErbB-2 (neu, HER2), ErbB-3 (HER3) and ErbB-4 (HER4) (Yarden et al., Nat. Rev. Mol. Cell. Biol, 2001, 2(2):127-137). The receptor proteins of this family are composed of an extracellular ligand-binding domain, a single hydrophobic transmembrane domain and a cytoplasmic tyrosine kinase-containing domain. There are at least 12 growth factors in the EGF family that bind to one or more of the ErbB receptors and effect receptor homo- or hetero-dimerization. Dimerization triggers internalization and recycling of the ligand-bound receptor (or its degradation), as well as downstream intracellular signaling pathways that regulate, inter alia, cell survival, apoptosis and proliferative activity. HER3 (ErbB3) is understood by those skilled in the art to lack tyrosine kinase activity.

EGFR, HER2 and recently HER3 have been associated with tumor formation. Recent studies have shown that EGFR is over expressed in a number of malignant human tissues when compared to their normal tissue counterparts. A high incidence of over-expression, amplification, deletion and structural rearrangement of the gene coding for EGFR has been found in tumors of the breast, lung, ovaries and kidney. For example, EGFR is overexpressed in 80% of head and neck cancers, activated by amplification and/or mutation in about 50% of glioblastomas, and activated by mutation in 10-15% of non-small cell lung carcinomas (NSCLCs) in the west and in 30-50% of NSCLCs in Asia (Frederick, L, Wang, X Y, Eley, G, James, C D (2000) Cancer Res 60: 1383-1387; Riely et al. (2006) Clin. Cancer Res. 12(24):7232-7241). Amplification of the EGFR gene in glioblastoma multiforme tumors is one of the most consistent genetic alterations known. EGFR overexpression has also been noted in many non-small cell lung carcinomas. HER2 is amplified or overexpressed in approximately 25-30% of breast cancers (Slamon et al. (1989) Science 244:707-712). Elevated levels of HER3 mRNA have been detected in human mammary carcinomas.

U.S. Pat. No. 6,277,640 to Bennett et al. discloses antisense compounds, compositions and methods for inhibiting the expression of HER3.

Several protein tyrosine kinase (“PTK”) inhibitors have been approved as selective therapies for certain cancers in which protein tyrosine kinase expression is dysregulated. Gleevec® (imatinib), which was initially approved in 2001, has been approved for the treatment of certain types leukemia in adults and children, aggressive systemic mastocytosis, hypereosinophilic syndrome, metastatic dermatofibrosarcoma protuberans, and certain types of metastatic malignant gastrointestinal stromal tumors. The small molecule PTK inhibitor Iressa® (gefitinib) has been approved for the treatment of locally advanced or metastatic non-small lung cancer after failure of platinum and docetaxel therapies. Tarceva™ (erlotinib) has been approved as a monotherapy for the treatment of locally advanced or metastatic non-small cell lung cancer or in combination with gemcitabine for the treatment of locally advanced, unresectable or metastatic pancreatic cancer. However, the efficacy of such therapies is limited because a resistance to the inhibitors develops over time. Arora et al. (2005) J. Pharmacol. and Exp. Therapies 315(3):971-971-979. Recently, it has been shown that inhibition of HER2 and EGFR tyrosine kinase activity using protein tyrosine kinase inhibitors show limited effect on HER2-driven breast cancers due to a compensatory increase in HER3 expression and subsequent signaling through the PI3K/Akt pathway (Sergina et al., Nature, 2007, 445:437-441).

There is a need for agents capable of effectively inhibiting HER3 function in cancers that are resistant to or have become less responsive to treatment with protein tyrosine kinase inhibitors and/or that have become resistant to or less responsive to treatment with HER2 inhibitors.

### 3. SUMMARY

In one embodiment, the invention provides methods of treating cancer in a mammal, comprising administering to the mammal an effective amount of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analogue; wherein the sequence of the first region is at least 80% identical to the reverse complement of the best-aligned target region of a mammalian HER3 gene or a mammalian HER3 mRNA; and wherein the cancer is resistant to treatment with a protein tyrosine kinase inhibitor and/or HER2 inhibitor and/or HER2 pathway inhibitor. Said resistance may be at least partially reversed as a result of reducing expression of HER3 using the oligomer. A related variation includes administering both the HER3 antisense oligomer and the protein tyrosine kinase inhibitor and/or HER2 inhibitor and/or HER2 pathway inhibitor such that the respective inhibitory effects of the oligomer and said inhibitor are temporally overlapping. In this manner, the invention provides treatments that at least partially prevent the development of resistance to such an inhibitor by a cancer (if not already developed) or at least partially reverse resistance to such an inhibitor by a cancer (if already developed).

The oligomer may, for example, have the sequence of SEQ ID NO: 180. The cancer may, for example, be a cancer resistant to treatment with gefitinib.

In some embodiments, the invention provides a method of treating cancer in a mammal, comprising administering to the mammal an effective amount of an oligomer consisting of the sequence 5′-TₛAₛGₛcₛcₛtₛgₛtₛcₛaₛcₛtₛtₛ^{Me}CₛTₛ^{Me}C-3′ (SEQ ID NO: 180), wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base, and wherein the cancer is resistant to treatment with a protein tyrosine kinase inhibitor such as but not limited to gefitinib or lapitinib.

In various embodiments, the invention provides a method of treating cancer in a mammal, comprising administering to the mammal an effective amount of a conjugate of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; wherein the sequence of the first region is at least 80% identical to the reverse complement of the best-aligned target region of a mammalian HER3 gene or a mammalian HER3 mRNA; and wherein the cancer is resistant to treatment with a protein tyrosine kinase inhibitor.

In certain embodiments, the invention provides a method of inhibiting the proliferation of a mammalian cancer cell comprising contacting the cell with an effective amount of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; wherein the sequence of the first region is at least 80% identical to the reverse complement of the best-aligned target region of a mammalian HER3 gene or a mammalian HER3 mRNA; and wherein proliferation of the mammalian cancer cell is not inhibited by a protein tyrosine kinase inhibitor.

Still another embodiment of the invention provides methods for treating cancers in a mammal by administering antisense oligomers that down-modulate (reduce) the expression of HER3 while, concurrently or in conjunction therewith, the mammal is treated with at least one protein tyrosine kinase inhibitor (PTKI) such as but not limited to gefitinb or any of those described herein. Said oligomers and PTKI may or may not be co-administered; what is important is that oligomers and PTKI are active together in therapeutically effective amounts in the mammal patient at the same time and/or the respective inhibitory effects of each are temporally overlapping. The cancers may be those that have been become resistant to or less responsive to treatment with PTKI, or they may be cancers which have never developed resistance to one or more PTKIs. The cancer may, for example, be a cancer at least initially responsive to treatment with one or more PTKIs, such as breast cancer, or may be any of the cancers described herein. Where the cancer is not substantially resistant to treatment with a PTKI, one embodiment provides for at least partially preventing resistance (or further resistance) to a PTKI by reducing the expression of HER3 in any of the manners described.

A related embodiment provides the use of at least one antisense oligomer that down-modulates (reduces) the expression of HER3 as described herein for the preparation of a medicament for use concurrently with or in conjunction with a PTKI, such as but not limited to gefitinib, in treating a cancer in a mammal, such as a cancer of a human patient, for example, breast cancer. Another embodiment provides the use of at least one oligomer that reduces the expression of HER3 in the preparation of a medicament for the treatment of a PTKI-resistant cancer in a mammal such as a human, for example, a PTKI-resistant human breast cancer patient. A further embodiment of the invention provides an improved method for treating a cancer in a mammal, such as a human patient, with at least one PTKI such as but not limited to gefitinib, in which the improvement comprises concurrently inhibiting the expression of HER3 in the mammal (e.g., in the cancer cells in the mammal), for example, by administering to the mammal at least one antisense oligomer that down-modulates the expression of HER3 such as those described herein. The at least one PTKI may, for example, be any of those described herein. The cancer may, for example, be a cancer at least initially responsive to a PTKI, such as breast cancer, or may be any of the cancers described herein.

In some embodiments, the proliferation of the mammalian cancer cell is inhibited by at least 50% when compared to the proliferation of an untreated cell of the same type.

Still another embodiment of the invention provides methods for treating cancers in a mammal by administering antisense oligomers that down-modulate the expression of HER3 while, concurrently or in conjunction therewith, the mammal is treated with at least one inhibitor of HER2 or of the HER2 pathway. Said oligomers and inhibitor of HER2 may or may not be co-administered; what is important is that oligomers and inhibitor of HER2 or HER2 pathway are active together in therapeutically effective amounts in the mammal patient at the same time and/or the respective inhibitory effects of each are temporally overlapping. The cancers may be those that have been become resistant to or less responsive to treatment with HER2 inhibitors, such as HER2-binding antibodies or binding fragments thereof, for example, trastuzumab or pertuzumab, or HER2 pathway inhibitors such as lapatinib, or they may be cancers which have never developed resistance to HER2 inhibitors. The cancer may, for example, be a cancer at least initially responsive to inhibition of HER2 or the HER2 pathway, such as breast cancer, or may be any of the cancers described herein. Where the cancer is not substantially resistant to treatment with a HER2 inhibitor or HER2 pathway inhibitor, one embodiment provides for at least partially preventing resistance (or further resistance) to a HER2 inhibitor or HER2 pathway inhibitor by reducing the expression of HER3 in any of the manners described.

A related embodiment provides the use of at least one antisense oligomer that down-modulates (reduces) the expression of HER3 as described herein for the preparation of a medicament for use concurrently with or in conjunction with at least one inhibitor of HER2 in treating a cancer in a mammal, such as a human patient. Another embodiment provides the use of at least one oligomer that reduces the expression of HER3 in the preparation of a medicament for the treatment of a cancer that has become resistant to or less responsive to treatment with an inhibitor of HER2 or the HER2 pathway, such as but not limited to trastuzumab or pertuzumab, or HER2 pathway inhibitors such as lapatinib, in a mammal such as a human, for example, a human with breast cancer that has become resistant to or less responsive to treatment with a HER2 inhibitor or inhibitor of the HER2 pathway. A further embodiment of the invention provides an improved method for treating a cancer in a mammal, such as a human patient, with an inhibitor of HER2 or the HER2 pathway, in which the improvement comprises concurrently inhibiting the expression of HER3 in the mammal (e.g., in the cancer cells in the mammal), for example, by administering to the mammal at least one antisense oligomer that down-modulates the expression of HER3 such as those described herein. The inhibitor of HER2 or the HER2 pathway may, for example, be any of those described herein. The cancer may, for example, be a cancer at least initially responsive to inhibition of HER2 or the HER2 pathway, such as breast cancer, or may be any of the cancers described herein.

For any of the aforementioned embodiments and variations thereof, the one or more antisense oligomers that reduce the expression of HER3 may, for example, be gapmers having terminal LNA monomers at each of the 5′ and 3′ ends, such as 1, 2, 3 or 4 contiguous LNA monomers at each end, which bound a central portion of DNA monomers. At least some, for example all, of the inter-monomer linkages maybe phosphorothioate linkages.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### 4. BRIEF DESCRIPTION OF THE FIGURES

FIG. 1. The HER3 target sequences that are targeted by the oligomers having the sequence of SEQ ID NOS: 1, 16, 17, 18, 19, 34, 49, 50, 51, 52, 53, 54, 55, 56, 57, 58, 59, 74, 75, 76, 91, 92, 107, 122, 137, 138, 139, and 140, respectively, are shown in bold and underlined, indicating their position in the HER3 transcript (GenBank Accession number NM_{—}001982-SEQ ID NO:197).

FIG. 2. HER3 mRNA expression in 15PC3, 24 hours after transfection, SEQ ID NOS:169-179

FIG. 3. EGFR mRNA expression in 15PC3, 24 hours after transfection, SEQ ID NOS:169-179

FIG. 4. HER-2 mRNA expression in 15PC3, 24 hours after transfection, SEQ ID NOS:169-179

FIG. 5: HER3 mRNA expression in 15PC3, 24 hours after transfection, SEQ ID NOS: 180-194

FIG. 6: Data show apoptosis induction measured as activated Caspase 3/7 at different time points in HUH7 cells transfected with oligonucleotides at 5 and 25 nM concentrations. Results are plotted relative to cells mock treated with a scrambled control oligonucleotide having SEQ ID NO: 235.

FIG. 7: Data show viable cells measured as OD490 using MTS assay at different time points in HUH-7 cells transfected with oligonucleotides at 5 and 25 nM concentrations. SEQ ID NO: 235 is a scrambled control oligonucleotide.

FIG. 8A: Data show percent change in tumor volume in 15PC3 xenograft tumors transplanted onto female nude mice treated with SEQ ID NO: 180 i.v. at 25 and 50 mg/kg q3d×10. Saline treated mice were used as control.

FIG. 8B: Data show HER3 mRNA expression in 15PC3 xenograft tumors transplanted onto female nude mice treated with SEQ ID NO: 180 i.v. at 25 and 50 mg/kg q3d×10. Results are normalized to GAPDH and presented as % of saline treated controls.

FIG. 9: Data show HER3 mRNA expression in mouse liver after treatment i.v. with 1 or 5 mg/kg oligonucleotides on three consecutive days having sequences shown in SEQ ID NO: 180 or SEQ ID NO: 234. Results are normalized to GAPDH and presented as % of saline treated controls.

FIG. 10: Data show the generation of HCC827 human lung adenocarcinoma cells that are resistant to gefitinib at a concentration as high as 10 μM.

FIG. 11: Data show that levels of phosphorylated EFGR are much lower in gefitinib-resistant HCC827 cells than in parent HCC827 gefitinib-sensitive cells.

FIG. 12: Data show that levels of unphosphorylated and phosphorylated EGFR are significantly reduced in HCC827 gefitinib-resistant clones, either in the presence (“+”) or absence (“−”) of gefitinib, as compared to the levels of unphosphorylated and phosphorylated EGFR in untreated (“−”) parent cells. In contrast, the levels of ErbB3 or MET, which are also involved in the EGFR signaling pathway, are not significantly decreased in the resistant clones compared to the parent cells.

FIG. 13: Data show that treatment with 1 μM of the oligonucleotide having SEQ ID NO: 180 over a 10-day period has a greater effect on inhibition of the growth of gefitinib-resistant HCC827 cells (greater than 80% reduction in growth as compared to untreated control) than on the growth of HCC827 cells that are sensitive to gefitinib.

FIG. 14: Data show that HER3 expression-reducing LNA antisense oligomer, but not trastuzumab, is able to prevent feedback upregulation of HER3 and P-HER3 expression by lapatinib in three human cancer cell lines.

FIG. 15: Data show that synergistic promotion of apoptosis in three human cancer cell lines is greater for a combination of lapatinib and a HER3 expression-reducing LNA antisense oligomer than for a combination of lapatinib and trastuzumab.

FIG. 16: Data show that antisense HER3 inhibitor SEQ ID NO: 180 inhibits tumor growth in an in vivo mouse xenograft model of human non-small cell lung cancer.

### 5. DETAILED DESCRIPTION

In certain embodiments, the invention provides methods for modulating the expression of HER3 (and/or EGFR and/or HER2) in cells that are resistant to treatment with a protein tyrosine kinase inhibitor. In some embodiments, the resistant cells are cancer cells. In various embodiments, methods are provided for treating or preventing diseases associated with HER3 over-expression, such as cancers that are resistant to treatment with protein tyrosine kinase inhibitors, by administering oligonucleotides (oligomers) which specifically hybridize under intracellular conditions to nucleic acids encoding. In some embodiments, oligonucleotides for use in the methods described herein down-regulate the expression of HER3. In other embodiments, oligonucleotides for use in the methods described herein down-regulate the expression of HER3, HER2 and/or EGFR.

The term “HER3” is used herein interchangeably with the term “ErbB3”.

5.1. Methods

In various embodiments, the invention encompasses methods of inhibiting the expression and/or activity of HER3 in a cell that is resistant to treatment with a protein tyrosine kinase inhibitor and/or HER2 or HER2 pathway inhibitor, comprising contacting the cell with an effective amount of an oligomeric compound (or a conjugate thereof) so as to effect the inhibition (e.g., down-regulation) of HER3 (and optionally one or more of HER2 and EGFR) expression and/or activity in a cell. In certain embodiments, HER3 (and optionally one or more of HER2 and EGFR) mRNA expression is inhibited. In other embodiments, HER3 (and optionally one or more of HER2 and EGFR) protein expression is inhibited. In various embodiments, the cell is a mammalian cell, such as a human cell. In various embodiments, the cell is a cancer cell.

In certain embodiments, the contacting occurs in vitro. In other embodiments, the contacting is effected in vivo by administering compositions as described herein to a mammal. In various embodiments, the invention provides a method of inhibiting (e.g., by down-regulating) the expression of HER3 protein and/or mRNA, and the expression of HER2 protein and/or mRNA in a cell. The sequence of the human HER2 mRNA is shown in SEQ ID NO: 199. In still further embodiments, the invention provides a method of inhibiting (e.g., by down-regulating) the expression of HER3 protein and/or mRNA in a cell, and the expression of EGFR protein and/or mRNA in a cell. The sequence of the human EGFR mRNA is shown in SEQ ID NO: 198. In yet further embodiments, the invention provides a method of inhibiting (e.g., by down-regulating) the expression of HER3, HER2 and EGFR mRNA and/or protein in a cell.

As used interchangeably herein, the terms “protein tyrosine kinase inhibitor,” “PTK inhibitor”, and “tyrosine kinase inhibitor” refer to molecules that bind to and inhibit the activity of one or more tyrosine kinase domains. The protein tyrosine kinase inhibitor is not the oligomer targeting HER3 as described herein below. In some embodiments the protein tyrosine kinase inhibitor is a monoclonal antibody. In other embodiments the protein tyrosine kinase inhibitor is a small molecule, having a molecular weight of less than 1000 Da, such as between 300-700 Da.

In certain embodiments, the PTK inhibitor is targeted to the tyrosine kinases of one or more EGFR family members. In various embodiments, the PTK inhibitor is targeted to the tyrosine kinases of one or more proteins that interact with or are regulated by one or more EGFR family members, e.g., proteins involved in one or more signaling cascades that originate with one or more EGFR family members. In some embodiments, the tyrosine kinase is a receptor tyrosine kinase, i.e., is an intra-cellular domain of a larger protein that has an extra-cellular ligand binding domain and is activated by the binding of one or more ligands. In certain embodiments, the protein tyrosine kinase is a non-receptor tyrosine kinase. Tyrosine kinase enzymes regulate the activities of other proteins in one or more signaling pathways by phosphorylating them.

As used herein, a cell that is resistant to treatment with a protein tyrosine kinase inhibitor refers to a cell whose growth or proliferation is not substantially reduced when contacted with a protein tyrosine kinase inhibitor. As used herein, the growth or proliferation of a cell is resistant to treatment with a PTK inhibitor if, when contacted with the PTK inhibitor, the growth or proliferation is reduced by less than 30%, such as by less than 20%, such as less than by 10%, as compared to the growth or proliferation of same type of cell that has not been contacted with the PTK inhibitor and lacks such resistance. In some embodiments, resistant cells are those that are inherently resistant to treatment with PTK inhibitors. In some embodiments, resistant cells are cells that have acquired resistance from prior exposure to a PTK inhibitor, either as a monotherapy or as part of a combination therapy with one or more additional agents, e.g., chemotherapeutic agents or antisense oligonucleotides. Similarly, as used herein, a cell that is resistant to treatment with a HER2 inhibitor, or HER2 pathway inhibitor generally, refers to a cell whose growth or proliferation is not substantially reduced when contacted with such an inhibitor. As used herein, the growth or proliferation of a cell is resistant to treatment with a HER2 inhibitor or HER2 pathway inhibitor if, when contacted with the inhibitor, the growth or proliferation is reduced by less than 30%, such as by less than 20%, such as less than by 10%, as compared to the growth or proliferation of same type of cell that has not been contacted with the inhibitor and lacks such resistance. In some embodiments, resistant cells are those that are inherently resistant to treatment with a HER2 inhibitor or HER2 pathway inhibitor. In some embodiments, resistant cells are cells that have acquired resistance from prior exposure to a HER2 inhibitor or HER2 pathway inhibitor.

In some embodiments, the cell has acquired resistance after having been exposed to a PTK inhibitor selected from gefitinib (ZD-1839, Iressa®), imatinib (Gleevec®), erlotinib (OSI-1774, Tarceva™), canertinib (CI-1033), vandetanib (ZD6474, Zactima®), tyrphostin AG-825 (CAS 149092-50-2), lapatinib (GW-572016), sorafenib (BAY43-9006), AG-494 (CAS 133550-35-3), RG-13022 (CAS 149286-90-8), RG-14620 (CAS 136831-49-7), BIBW 2992 (Tovok), tyrphostin 9 (CAS 136831-49-7), tyrphostin 23 (CAS 118409-57-7), tyrphostin 25 (CAS 118409-58-8), tyrphostin 46 (CAS 122520-85-8), tyrphostin 47 (CAS 122520-86-9), tyrphostin 53 (CAS 122520-90-5), butein (1-(2,4-dihydroxyphenyl)-3-(3,4-dihydroxyphenyl)-2-propen-1-one 2′,3,4,4′-Tetrahydroxychalcone; CAS 487-52-5), curcumin ((E,E)-1,7-bis(4-Hydroxy-3-methoxyphenyl)-1,6-heptadiene-3,5-dione; CAS 458-37-7), N4-(1-Benzyl-1H-indazol-5-yl)-N6,N6-dimethyl-pyrido-[3,4-d]-pyrimidine-4,6-diamine (202272-68-2), AG-1478, AG-879, Cyclopropanecarboxylic acid-(3-(6-(3-trifluoromethyl-phenylamino)-pyrimidin-4-ylamino)-phenyl)-amide (CAS 879127-07-8), N8-(3-Chloro-4-fluorophenyl)-N2-(1-methylpiperidin-4-yl)-pyrimido[5,4-d]pyrimidine-2,8-diamine, 2HCl (CAS 196612-93-8), 4-(4-Benzyloxyanilino)-6,7-dimethoxyquinazoline (CAS 179248-61-4), N-(4-((3-Chloro-4-fluorophenyl)amino)pyrido[3,4-d]pyrimidin-6-yl)-2-butynamide (CAS 881001-19-0), EKB-569, HKI-272, and HKI-357.

In various embodiments, the cell has acquired resistance after having been exposed to a PTK inhibitor selected from gefitinib, imatinib, erlotinib, lapatinib, canertinib and sorafenib. In one variation, the cell has acquired resistance after having been exposed to gefitinib.

In certain embodiments, the cell has acquired resistance after having been exposed to HER2 inhibitor such as a HER2-binding and -inhibiting antibody or -binding and -inhibiting antibody fragment. In one variation, the cell has acquired resistance after having been exposed to trastuzumab and/or pertuzumab.

In certain embodiments, the invention relates to a method of treating a disease in a patient, wherein the disease is resistant to treatment with a PTK inhibitor and/or HER2 or HER2 pathway inhibitor, comprising administering to a patient in need thereof a pharmaceutical composition comprising an effective amount of at least one oligomer, or a conjugate thereof, and a pharmaceutically acceptable excipient. As used herein, the terms “treating” and “treatment” refer to both treatment of an existing disease (e.g., a disease or disorder as referred to herein below), or prevention of a disease, i.e., prophylaxis.

In certain embodiments, the methods of the invention are useful for inhibiting proliferation of cells that are resistant to PTK inhibitor(s) and/or HER2 and/or HER2 pathway inhibitor(s). In various embodiments the anti-proliferative effect is an at least 10% reduction, an at least 20% reduction, an at least 30% reduction, an at least 40% reduction, an at least 50% reduction, an at least 60% reduction, an at least 70% reduction, an at least 80% reduction, or an at least 90% reduction in cell proliferation as compared to a cell sample that is untreated. In other embodiments, the anti-proliferative effect is an at least 10% reduction, an at least 20% reduction, an at least 30% reduction, an at least 40% reduction, an at least 50% reduction, an at least 60% reduction, an at least 70% reduction, an at least 80% reduction, or an at least 90% reduction in cell proliferation as compared to a cell sample that is treated with a small molecule protein tyrosine kinase inhibitor. In various embodiments, the cell is a cancer cell. In some embodiments, the cancer cell is selected from a breast cancer cell, a prostate cancer cell, a lung cancer cell, and an epithelial carcinoma cell.

Accordingly, the methods of the invention are useful for treating a hyperproliferative disease, such as cancer, which is resistant to treatment with a protein tyrosine kinase inhibitor and/or to treatment with a HER2 or HER2 pathway inhibitor. In some embodiments, the resistant cancer to be treated is selected from the group consisting of lymphomas and leukemias (e.g. non-Hodgkin's lymphoma, Hodgkin's lymphoma, acute leukemia, acute lymphocytic leukemia, acute myelocytic leukemia, chronic myeloid leukemia, chronic lymphocytic leukemia, multiple myeloma), colon carcinoma, rectal carcinoma, epithelial carcinoma, pancreatic cancer, breast cancer, ovarian cancer, prostate cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma, heavy chain disease, metastases, or any disease or disorder characterized by uncontrolled or abnormal cell growth.

In certain embodiments, the resistant cancer is selected from the group consisting of lung cancer, prostate cancer, breast cancer, ovarian cancer, colon cancer, epithelial carcinoma, and stomach cancer.

In certain other embodiments, the lung cancer is non-small cell lung cancer. One such embodiment of the invention provides a method for the treatment of non-small cell lung cancer that includes administering to a mammal such as a human patient in need of treatment for said cancer, a therapeutically effective amount of at least one antisense oligomer or a conjugate thereof that reduces the expression of HER3 and optionally one or more inhibitors of HER2 or the HER2 pathway. In one variation, the at least one oligomer or conjugate thereof includes or is SEQ ID NO: 180 or a conjugate thereof

In certain embodiments, the invention also provides for the use of the compounds or conjugates described herein for the manufacture of a medicament for the treatment of a PTK inhibitor-resistant, HER2 inhibitor-resistant or HER2 pathway inhibitor-resistant disorder as referred to herein, or for a method of the treatment of such a disorder as referred to herein.

In various embodiments, the treatment of PTK inhibitor-resistant, HER2 inhibitor-resistant or HER2 pathway inhibitor-resistant disorders according to the invention may be combined with one or more other anti-cancer treatments, such as radiotherapy, chemotherapy or immunotherapy.

In certain embodiments, the PTK inhibitor-resistant disease is associated with a mutation in the HER3 gene (and/or the HER2 gene and/or the EGFR gene) or a gene whose protein product is associated with or interacts with HER3. In some embodiments, the mutated gene codes for a protein with a mutation in the tyrosine kinase domain. In various embodiments, the mutation in the tyrosine kinase domain is in the binding site of a small molecule PTK inhibitor and/or the ATP binding site. Therefore, in various embodiments, the target mRNA is a mutated form of the HER3 (and/or HER2 and/or EGFR) sequence; for example, it comprises one or more single point mutations, such as SNPs associated with cancer.

In certain embodiments, the PTK inhibitor-resistant disease is associated with abnormal levels of a mutated form of HER3. In certain embodiments, the PTK inhibitor-resistant disease is associated with abnormal levels of a wild-type form of HER3. One aspect of the invention is directed to a method of treating a patient suffering from or susceptible to conditions associated with abnormal levels of HER3, comprising administering to the patient a therapeutically effective amount of an oligomer targeted to HER3 or a conjugate thereof. In some embodiments, the oligomer comprises one or more LNA units as described herein below.

In various embodiments, the invention is directed to a method of treating a patient suffering from or susceptible to conditions associated with abnormal levels of a mutated form of HER2, or abnormal levels of a wild-type form of HER2, wherein the condition is resistant to treatment with a protein tyrosine kinase inhibitor, comprising administering to the mammal a therapeutically effective amount of an oligomer targeted to HER3 (and optionally to one or more of HER2 and EGFR) or a conjugate thereof. In some embodiments, the oligomer comprises one or more LNA units as described herein below.

In still other embodiments, the invention is directed to a method of treating a patient suffering from or susceptible to conditions associated with abnormal levels of a mutated EGFR, or abnormal levels of a wild-type EGFR, wherein the condition is resistant to treatment with a protein tyrosine kinase inhibitor, comprising administering to the patient a therapeutically effective amount of an oligomer targeted to HER3 (and optionally to one or more of HER2 and EGFR) or a conjugate thereof. In some embodiments, the oligomer comprises one or more LNA units as described herein below.

In various embodiments, the invention described herein encompasses a method of preventing or treating a disease that is resistant to treatment with a protein tyrosine kinase inhibitor comprising administering to a human in need of such therapy a therapeutically effective amount a HER3 modulating oligomer (and optionally one or more of HER2 and EGFR) or a conjugate thereof.

In various embodiments, the oligomer, or conjugate thereof, induces a desired therapeutic effect in humans through, for example, hydrogen bonding to a target nucleic acid. The oligomer causes a decrease (e.g., inhibition) in the expression of a target via hydrogen bonding (e.g., hybridization) to the mRNA of the target thereby resulting in a reduction in gene expression.

It is highly preferred that the compounds of the invention are capable of hybridizing to the target nucleic acid, such as HER3 mRNA, by Watson-Crick base pairing.

5.2. Oligomers

In a first aspect, oligomeric compounds (referred to herein as oligomers), are provided that are useful, e.g., in modulating the function of nucleic acid molecules encoding mammalian HER3, such as the HER3 nucleic acid shown in SEQ ID No: 197, and naturally occurring allelic variants of such nucleic acid molecules encoding mammalian HER3. The oligomers are composed of covalently linked monomers.

The term “monomer” includes both nucleosides and deoxynucleosides (collectively, “nucleosides”) that occur naturally in nucleic acids and that do not contain either modified sugars or modified nucleobases, i.e., compounds in which a ribose sugar or deoxyribose sugar is covalently bonded to a naturally-occurring, unmodified nucleobase (base) moiety (i.e., the purine and pyrimidine heterocycles adenine, guanine, cytosine, thymine or uracil) and “nucleoside analogues,” which are nucleosides that either do occur naturally in nucleic acids or do not occur naturally in nucleic acids, wherein either the sugar moiety is other than a ribose or a deoxyribose sugar (such as bicyclic sugars or 2′ modified sugars, such as 2′ substituted sugars), or the base moiety is modified (e.g., 5-methylcytosine), or both.

An “RNA monomer” is a nucleoside containing a ribose sugar and an unmodified nucleobase.

A “DNA monomer” is a nucleoside containing a deoxyribose sugar and an unmodified nucleobase.

A “Locked Nucleic Acid monomer,” “locked monomer,” or “LNA monomer” is a nucleoside analogue having a bicyclic sugar, as further described herein below.

The terms “corresponding nucleoside analogue” and “corresponding nucleoside” indicate that the base moiety in the nucleoside analogue and the base moiety in the nucleoside are identical. For example, when the “nucleoside” contains a 2-deoxyribose sugar linked to an adenine, the “corresponding nucleoside analogue” contains, for example, a modified sugar linked to an adenine base moiety.

The terms “oligomer,” “oligomeric compound,” and “oligonucleotide” are used interchangeably in the context of the methods described herein, and refer to a molecule formed by covalent linkage of two or more contiguous monomers by, for example, a phosphate group (forming a phosphodiester linkage between nucleosides) or a phosphorothioate group (forming a phosphorothioate linkage between nucleosides). The oligomer consists of, or comprises, 10-50 monomers, such as 10-30 monomers.

In some embodiments, an oligomer comprises nucleosides, or nucleoside analogues, or mixtures thereof as referred to herein. An “LNA oligomer” or “LNA oligonucleotide” refers to an oligonucleotide containing one or more LNA monomers.

Nucleoside analogues that are optionally included within oligomers may function similarly to corresponding nucleosides, or may have specific improved functions. Oligomers wherein some or all of the monomers are nucleoside analogues are often preferred over native forms because of several desirable properties of such oligomers, such as the ability to penetrate a cell membrane, good resistance to extra- and/or intracellular nucleases and high affinity and specificity for the nucleic acid target. LNA monomers are particularly preferred, for example, for conferring several of the above-mentioned properties.

In various embodiments, one or more nucleoside analogues present within the oligomer are “silent” or “equivalent” in function to the corresponding natural nucleoside, i.e., have no functional effect on the way the oligomer functions to inhibit target gene expression. Such “equivalent” nucleoside analogues are nevertheless useful if, for example, they are easier or cheaper to manufacture, or are more stable under storage or manufacturing conditions, or can incorporate a tag or label. Typically, however, the analogues will have a functional effect on the way in which the oligomer functions to inhibit expression; for example, by producing increased binding affinity to the target region of the target nucleic acid and/or increased resistance to intracellular nucleases and/or increased ease of transport into the cell.

Thus, in various embodiments, oligomers for use in the methods of the invention comprise nucleoside monomers and at least one nucleoside analogue monomer, such as an LNA monomer, or other nucleoside analogue monomers.

The term “at least one” comprises the integers larger than or equal to 1, such as 1, 2, 3, 4, 5, 6, 7, 8, 9, 10, 11, 12, 13, 14, 15, 16, 17, 18, 19, 20 and so forth. In various embodiments, such as when referring to the nucleic acid or protein targets of the compounds of the invention, the term “at least one” includes the terms “at least two” and “at least three” and “at least four.” Likewise, in some embodiments, the term “at least two” comprises the terms “at least three” and “at least four.”

In some embodiments, the oligomer consists of 10-50 contiguous monomers, such as 10, 11, 12, 13, 14, 15, 16, 17, 18, 19, 20, 21, 22, 23, 24, 25, 26, 27, 28, 29 or 30 contiguous monomers.

In some embodiments, the oligomer consists of 10-25 monomers, preferably, 10-16 monomers, and more preferably, 12-16 monomers.

In various embodiments, the oligomers comprise or consist of 10-25 contiguous monomers, 10-24 contiguous monomers, 12-25 or 12-24 or 10-22 contiguous monomers, such as 12-18 contiguous monomers, such as 13-17 or 12-16 contiguous monomers, such as 13, 14, 15, 16 contiguous monomers.

In various embodiments, the oligomers comprise or consist of 10-22 contiguous monomers, or 10-18, such as 12-18 or 13-17 or 12-16, such as 13, 14, 15 or 16 contiguous monomers.

In some embodiments, the oligomers comprise or consist of 10-16 or 12-16 or 12-14 contiguous monomers. In other embodiments, the oligomers comprise or consist of 14-18 or 14-16 contiguous monomers.

In various embodiments, the oligomers comprise or consist of 10, 11, 12, 13, or 14 contiguous monomers.

In various embodiments, the oligomer consists of no more than 22 contiguous monomers, such as no more than 20 contiguous monomers, such as no more than 18 contiguous monomers, such as 15, 16 or 17 contiguous monomers. In certain embodiments, the oligomer comprises less than 20 contiguous monomers.

In various embodiments, the oligomer does not comprise RNA monomers.

It is preferred that the oligomers for use in the methods described herein are linear molecules or are linear as synthesized. The oligomer is, in such embodiments, a single stranded molecule, and typically does not comprise a short region of, for example, at least 3, 4 or 5 contiguous monomers, which are complementary to another region within the same oligomer such that the oligomer forms an internal duplex. In various embodiments, the oligomer is not substantially double-stranded, i.e., is not a siRNA.

In some embodiments, the oligomer consists of a contiguous stretch of monomers, the sequence of which is identified by a SEQ ID NO. disclosed herein (see, e.g., Tables 1-4). In other embodiments, the oligomer comprises a first region, the region consisting of a contiguous stretch of monomers, and one or more additional regions which consist of at least one additional monomer. In some embodiments, the sequence of the first region is identified by a SEQ ID NO. disclosed herein.

5.3. Gapmer Design

Typically, the oligomer for use in the methods of the invention is a gapmer.

A “gapmer” is an oligomer which comprises a contiguous stretch of monomers capable of recruiting an RNAse (e.g. RNAseH) as further described herein below, such as a region of at least 6 or 7 DNA monomers, referred to herein as region B, wherein region B is flanked both on its 5′ and 3′ ends by regions respectively referred to as regions A and C, each of regions A and C comprising or consisting of nucleoside analogues, such as affinity-enhancing nucleoside analogues, such as 1-6 nucleoside analogues.

Typically, the gapmer comprises regions, from 5′ to 3′, A-B-C, or optionally A-B-C-D or D-A-B-C, wherein: region A consists of or comprises at least one nucleoside analogue, such as at least one LNA monomer, such as 1-6 nucleoside analogues, such as LNA monomers; and region B consists of or comprises at least five contiguous monomers which are capable of recruiting RNAse (when formed in a duplex with a complementary target region of the target RNA molecule, such as the mRNA target), such as DNA monomers; and region C consists of or comprises at least one nucleoside analogue, such as at least one LNA monomer, such as 1-6 nucleoside analogues, such as LNA monomers, and; region D, when present, consists of or comprises 1, 2 or 3 monomers, such as DNA monomers.

In various embodiments, region A consists of 1, 2, 3, 4, 5 or 6 nucleoside analogues, such as LNA monomers, such as 2-5 nucleoside analogues, such as 2-5 LNA monomers, such as 3 or 4 nucleoside analogues, such as 3 or 4 LNA monomers; and/or region C consists of 1, 2, 3, 4, 5 or 6 nucleoside analogues, such as LNA monomers, such as 2-5 nucleoside analogues, such as 2-5 LNA monomers, such as 3 or 4 nucleoside analogues, such as 3 or 4 LNA monomers.

In certain embodiments, region B consists of or comprises 5, 6, 7, 8, 9, 10, 11 or 12 contiguous monomers which are capable of recruiting RNAse, or 6-10, or 7-9, such as 8 contiguous monomers which are capable of recruiting RNAse. In certain embodiments, region B consists of or comprises at least one DNA monomer, such as 1-12 DNA monomers, preferably 4-12 DNA monomers, more preferably 6-10 DNA monomers, such as 7-10 DNA monomers, most preferably 8, 9 or 10 DNA monomers.

In certain embodiments, region A consists of 3 or 4 nucleoside analogues, such as LNA monomers, region B consists of 7, 8, 9 or 10 DNA monomers, and region C consists of 3 or 4 nucleoside analogues, such as LNA monomers. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 monomers, such as DNA monomers.

Further gapmer designs are disclosed in WO 2004/046160, which is hereby incorporated by reference.

U.S. provisional application, 60/977,409, hereby incorporated by reference, refers to “shortmer” gapmer oligomers. In some embodiments, oligomers presented here may be such shortmer gapmers.

In certain embodiments, the oligomer consists of 10, 11, 12, 13 or 14 contiguous monomers, wherein the regions of the oligomer have the pattern (5′-3′), A-B-C, or optionally A-B-C-D or D-A-B-C, wherein; region A consists of 1, 2 or 3 nucleoside analogue monomers, such as LNA monomers; region B consists of 7, 8 or 9 contiguous monomers which are capable of recruiting RNAse when formed in a duplex with a complementary RNA molecule (such as a mRNA target); and region C consists of 1, 2 or 3 nucleoside analogue monomers, such as LNA monomers. When present, region D consists of a single DNA monomer.

In certain embodiments, region A consists of 1 LNA monomer. In certain embodiments, region A consists of 2 LNA monomers. In certain embodiments, region A consists of 3 LNA monomers. In certain embodiments, region C consists of 1 LNA monomer. In certain embodiments, region C consists of 2 LNA monomers. In certain embodiments, region C consists of 3 LNA monomers. In certain embodiments, region B consists of 7 nucleoside monomers. In certain embodiments, region B consists of 8 nucleoside monomers. In certain embodiments, region B consists of 9 nucleoside monomers. In certain embodiments, region B comprises 1-9 DNA monomers, such as 2, 3, 4, 5, 6, 7 or 8 DNA monomers. In certain embodiments, region B consists of DNA monomers. In certain embodiments, region B comprises at least one LNA monomer which is in the alpha-L configuration, such as 2, 3, 4, 5, 6, 7, 8 or 9 LNA monomers in the alpha-L-configuration. In certain embodiments, region B comprises at least one alpha-L-oxy LNA monomer. In certain embodiments, all the LNA monomers in region B that are in the alpha-L-configuration are alpha-L-oxy LNA monomers. In certain embodiments, the number of monomers present in the A-B-C regions of the oligomers is selected from the group consisting of (nucleotide analogue monomers-region B-nucleoside analogue monomers): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, and 3-10-1. In certain embodiments, the number of monomers present in the A-B-C regions of the oligomers described herein is selected from the group consisting of: 2-7-1, 1-7-2, 2-7-2, 3-7-3, 2-7-3, 3-7-2, 3-7-4, and 4-7-3. In certain embodiments, each of regions A and C consists of two LNA monomers, and region B consists of 8 or 9 nucleoside monomers, preferably DNA monomers.

In various embodiments, other gapmer designs include those where regions A and/or C consists of 3, 4, 5 or 6 nucleoside analogues, such as monomers containing a 2′-O-methoxyethyl-ribose sugar (2′MOE) or monomers containing a 2′-fluoro-deoxyribose sugar, and region B consists of 8, 9, 10, 11 or 12 nucleosides, such as DNA monomers, where regions A-B-C have 5-10-5 or 4-12-4 monomers. Further gapmer designs are disclosed in WO 2007/146511A2, hereby incorporated by reference.

5.4. Linkage Groups

The monomers of the oligomers described herein are coupled together via linkage groups. Suitably, each monomer is linked to the 3′ adjacent monomer via a linkage group.

The terms “linkage group” or “internucleoside linkage” mean a group capable of covalently coupling together two contiguous monomers. Specific and preferred examples include phosphate groups (forming a phosphodiester between adjacent nucleoside monomers) and phosphorothioate groups (forming a phosphorothioate linkage between adjacent nucleoside monomers).

Suitable linkage groups include those listed in WO 2007/031091, for example the linkage groups listed on the first paragraph of page 34 of WO 2007/031091 (hereby incorporated by reference).

It is, in various embodiments, preferred to modify the linkage group from its normal phosphodiester to one that is more resistant to nuclease attack, such as phosphorothioate or boranophosphate—these two, being cleavable by RNase H, permitting RNase-mediated antisense inhibition of expression of the target gene.

In some embodiments, suitable sulphur (S) containing linkage groups as provided herein are preferred. In various embodiments, phosphorothioate linkage groups are preferred, particularly for the gap region (B) of gapmers. In certain embodiments, phosphorothioate linkages are used to link together monomers in the flanking regions (A and C). In various embodiments, phosphorothioate linkages are used for linking regions A or C to region D, and for linking together monomers within region D.

In various embodiments, regions A, B and C comprise linkage groups other than phosphorothioate, such as phosphodiester linkages, particularly, for instance when the use of nucleoside analogues protects the linkage groups within regions A and C from endo-nuclease degradation—such as when regions A and C comprise LNA monomers.

In various embodiments, adjacent monomers of the oligomer are linked to each other by means of phosphorothioate groups.

It is recognized that the inclusion of phosphodiester linkages, such as one or two linkages, into an oligomer with a phosphorothioate backbone, particularly with phosphorothioate linkage groups between or adjacent to nucleoside analogue monomers (typically in region A and/or C), can modify the bioavailability and/or bio-distribution of an oligomer—see WO 2008/053314, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleoside linkage groups are phosphorothioate.

When referring to specific gapmer oligonucleotide sequences, such as those provided herein, it will be understood that, in various embodiments, when the linkages are phosphorothioate linkages, alternative linkages, such as those disclosed herein, may be used, for example phosphate (phosphodiester) linkages may be used, particularly for linkages between nucleoside analogues, such as LNA monomers.

5.5. Target Nucleic Acid

The terms “nucleic acid” and “polynucleotide” are used interchangeably herein, and are defined as a molecule formed by covalent linkage of two or more monomers, as above-described. Including 2 or more monomers, “nucleic acids” may be of any length, and the term is generic to “oligomers”, which have the lengths described herein. The terms “nucleic acid” and “polynucleotide” include single-stranded, double-stranded, partially double-stranded, and circular molecules.

In various embodiments, the term “target nucleic acid,” as used herein, refers to the nucleic acid (such as DNA or RNA) encoding mammalian HER3 polypeptide (e.g., such as human HER3 mRNA having the sequence in SEQ ID NO 197, or mammalian mRNAs having GenBank Accession numbers NM_{—}001005915, NM_{—}001982 and alternatively-spliced forms NP_{—}001973.2 and NP_{—}001005915.1 (human); NM_{—}017218 (rat); NM_{—}010153 (mouse); NM_{—}001103105 (cow); or predicted mRNA sequences having GenBank Accession numbers XM_{—}001491896 (horse), XM_{—}001169469 and XM_{—}509131 (chimpanzee)).

In various embodiments, “target nucleic acid” also includes a nucleic acid encoding a mammalian HER2 polypeptide (e.g., such mammalian mRNAs having GenBank Accession numbers NM_{—}001005862 and NM_{—}004448 (human); NM_{—}017003 and NM_{—}017218 (rat); NM_{—}001003817 (mouse); NM_{—}001003217 (dog); and NM_{—}001048163 (cat)).

In various embodiments, “target nucleic acid” also includes a nucleic acid encoding a mammalian EGFR polypeptide (e.g., such as mammalian mRNAs having GenBank Accession numbers NM_{—}201284, NM_{—}201283, NM_{—}201282 and NM_{—}005228 (human); NM_{—}007912 and NM_{—}207655 (mouse); NM_{—}031507 (rat); and NM_{—}214007 (pig)).

It is recognized that the above-disclosed GenBank Accession numbers refer to cDNA sequences and not to mRNA sequences per se. The sequence of a mature mRNA can be derived directly from the corresponding cDNA sequence, with thymine bases (T) being replaced by uracil bases (U).

In various embodiments, “target nucleic acid” also includes HER3 (or HER2 or EGFR) encoding nucleic acids or naturally occurring variants thereof, and RNA nucleic acids derived therefrom, preferably mRNA, such as pre-mRNA, although preferably mature mRNA. In various embodiments, for example when used in research or diagnostics the “target nucleic acid” is a cDNA or a synthetic oligonucleotide derived from the above DNA or RNA target nucleic acids. The oligomers described herein are typically capable of hybridizing to the target nucleic acid.

The term “naturally occurring variant thereof” refers to variants of the HER3 (or HER2 or EGFR) polypeptide or nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the HER3 (or HER2 or EGFR) encoding genomic DNA which is found at the Chromosome Chr 12: 54.76-54.78 Mb by chromosomal translocation or duplication, and the RNA, such as mRNA derived therefrom. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

In certain embodiments, oligomers described herein bind to a region of the target nucleic acid (the “target region”) by either Watson-Crick base pairing, Hoogsteen hydrogen bonding, or reversed Hoogsteen hydrogen bonding, between the monomers of the oligomer and monomers of the target nucleic acid. Such binding is also referred to as “hybridization.” Unless otherwise indicated, binding is by Watson-Crick pairing of complementary bases (i.e., adenine with thymine (DNA) or uracil (RNA), and guanine with cytosine), and the oligomer binds to the target region because the sequence of the oligomer is identical to, or partially-identical to, the sequence of the reverse complement of the target region; for purposes herein, the oligomer is said to be “complementary” or “partially complementary” to the target region, and the percentage of “complementarity” of the oligomer sequence to that of the target region is the percentage “identity” to the reverse complement of the sequence of the target region.

Unless otherwise made clear by context, the “target region” herein will be the region of the target nucleic acid having the sequence that best aligns with the reverse complement of the sequence of the specified oligomer (or region thereof), using the alignment program and parameters described herein below.

In determining the degree of “complementarity” between oligomers for use in the methods described herein (or regions thereof) and the target region of the nucleic acid which encodes mammalian HER3 (or HER2 or EGFR), such as those disclosed herein, the degree of “complementarity” (also, “homology”) is expressed as the percentage identity between the sequence of the oligomer (or region thereof) and the reverse complement of the sequence of the target region that best aligns therewith. The percentage is calculated by counting the number of aligned bases that are identical as between the 2 sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer and the target region.

Amino acid and polynucleotide alignments, percentage sequence identity, and degree of complementarity may be determined for purposes of the invention using the ClustalW algorithm using standard settings: see http://www.ebi.ac.uk/emboss/align/index.html, Method: EMBOSS::water (local): Gap Open=10.0, Gap extend=0.5, using Blosum 62 (protein), or DNAfull for nucleotide/nucleobase sequences.

As will be understood, depending on context, “mismatch” refers to a nonidentity in sequence (as, for example, between the nucleobase sequence of an oligomer and the reverse complement of the target region to which it binds; as for example, between the base sequence of two aligned HER3 encoding nucleic acids), or to noncomplementarity in sequence (as, for example, between an oligomer and the target region to which binds).

Suitably, the oligomer (or conjugate, as further described, below) is capable of inhibiting (such as, by down-regulating) expression of the HER3 (or HER2 or EGFR) gene.

In various embodiments, the oligomers described herein effect inhibition of HER3 (or HER2 or EGFR) mRNA expression of at least 10% as compared to the normal expression level, at least 20%, more preferably at least 30%, 40%, 50%, 60%, 70%, 80%, 90% or 95% as compared to the normal expression level. In various embodiments, the oligomers effect inhibition of HER3 (or HER2 or EGFR) protein expression of at least 10% as compared to the normal expression level, at least 20%, more preferably at least 30%, 40%, 50%, 60%, 70%, 80%, 90% or 95% as compared to the normal expression level. In some embodiments, such inhibition is seen when using 1 nM of the oligomer or conjugate for use in the methods of the invention. In various embodiments, such inhibition is seen when using 25 nM of the oligomer or conjugate.

In various embodiments, the inhibition of mRNA expression is less than 100% (i.e., less than complete inhibition of expression), such as less than 98%, inhibition, less than 95% inhibition, less than 90% inhibition, less than 80% inhibition, such as less than 70% inhibition. In various embodiments, the inhibition of protein expression is less than 100% (i.e., less than complete inhibition of expression), such as less than 98%, inhibition, less than 95% inhibition, less than 90% inhibition, less than 80% inhibition, such as less than 70% inhibition.

Alternatively, modulation of expression levels can be determined by measuring levels of mRNA, e.g. by northern blotting or quantitative RT-PCR. When measuring via mRNA levels, the level of inhibition when using an appropriate dosage, such as 1 and 25 nM, is, in various embodiments, typically to a level of 10-20% of the normal levels in the absence of the compound.

Modulation (i.e., inhibition or increase) of expression level may also be determined by measuring protein levels, e.g. by methods such as SDS-PAGE followed by western blotting using suitable antibodies raised against the target protein.

In some embodiments, the invention provides oligomers that inhibit (e.g., down-regulate) the expression of one or more alternatively-spliced isoforms of HER3 mRNA and/or proteins derived therefrom. In some embodiments, the invention provides oligomers that inhibit expression of one or more of the alternatively-spliced protein isoforms of HER3 (GenBank Accession nos. NP_{—}001973.2 and NP_{—}001005915.1) and/or expression of the nucleic acids that encode the HER3 protein isoforms (GenBank Accession nos. NM_{—}001982 and NM_{—}001005915.1). In some embodiments, the mRNA encoding HER3 isoform 1 is the target nucleic acid. In other embodiments, the mRNA encoding HER3 isoform 2 is the target nucleic acid. In certain embodiments, the nucleic acids encoding HER3 isoform 1 and HER3 isoform 2 are target nucleic acids, for example, the oligomer having the sequence of SEQ ID NO: 180.

In various embodiments, oligomers, or a first region thereof, have a base sequence that is complementary to the sequence of a target region in a HER3 nucleic acid, which oligomers down-regulate HER3 mRNA and/or HER3 protein expression and down-regulate the expression of mRNA and/or protein of one or more other ErbB receptor tyrosine kinase family members, such as HER2 and/or EGFR. Oligomers, or a first region thereof, that effectively bind to the target regions of two different ErbB receptor family nucleic acids (e.g., HER2 and HER3 mRNA) and that down-regulating the mRNA and/or protein expression of both targets are termed “bispecific.” Oligomers, or a first region thereof, that bind to the target regions of three different ErbB receptor family members and are capable of effectively down-regulating all three genes are termed “trispecific”. In various embodiments, an antisense oligonucleotide may be polyspecific, i.e. capable of binding to target regions of target nucleic acids of multiple members of the ErbB family of receptor tyrosine kinases and down-regulating their expression. As used herein, the terms “bispecific” and “trispecific” are understood not to be limiting in any way. For example, a “bispecific oligomer” may have some effect on a third target nucleic acid, while a “trispecific oligomer” may have a very weak and therefore insignificant effect on one of its three target nucleic acids.

In various embodiments, bispecific oligomers, or a first region thereof, are capable of binding to a target region in a HER3 nucleic acid and a target region in a HER2 target nucleic acid and effectively down-regulating the expression of HER3 and HER2 mRNA and/or protein. In certain embodiments, the bispecific oligomers do not down-regulate expression of HER3 mRNA and/or protein and HER2 mRNA and/or protein to the same extent. In other preferred embodiments, the bispecific oligomers, or a first region thereof, are capable of binding to a target region in a HER3 target nucleic acid and a target region in an EGFR target nucleic acid and effectively down-regulating the expression of HER3 mRNA and/or protein and EGFR mRNA and/or protein. In various embodiments, the bispecific oligomers do not down-regulate expression of HER3 mRNA and/or protein and EGFR mRNA and/or protein to the same extent. In still other embodiments, trispecific oligomers, or a first region thereof, are capable of binding to a target region in a HER3 target nucleic acid, and to target regions in two other ErbB family of receptor tyrosine kinase target nucleic acids and effectively down-regulating the expression of HER3 mRNA and/or protein and mRNA and/or protein of the two other members of the ErbB family of receptor tyrosine kinases. In various preferred embodiments, the trispecific oligomers, or a first region thereof, are capable of effectively down-regulating the expression of HER3 mRNA and/or protein, the expression of HER2 mRNA and/or protein, and the expression of EGFR mRNA and/or protein. In various embodiments, the trispecific oligomers do not down-regulate expression of HER3 mRNA and/or protein, HER2 mRNA and/or protein and EGFR mRNA and/or protein to the same extent.

In various embodiments, the invention therefore provides a method of inhibiting (e.g., by down-regulating) the expression of HER3 protein and/or mRNA in a cancer cell which is expressing HER3 protein and/or mRNA and which is resistant to treatment with a protein tyrosine kinase inhibitor, the method comprising contacting the cell with an amount of an oligomer or conjugate as described herein effective to inhibit (e.g., to down-regulate) the expression of HER3 protein and/or mRNA in said cell. Suitably the cell is a mammalian cell, such as a human cell. The contacting may occur, in certain embodiments, in vitro. In other embodiments, the contacting may be effected in vivo, by administering the compound or conjugate described herein to a mammal. In various embodiments, the invention provides a method of inhibiting (e.g., by down-regulating) the expression of HER3 protein and/or mRNA and the expression of HER2 protein and/or mRNA in a cell that is resistant to treatment with a protein tyrosine kinase inhibitor. The sequence of the human HER2 mRNA is shown in SEQ ID NO: 199. In still further embodiments, the invention provides a method of inhibiting (e.g., by down-regulating) the expression of HER3 protein and/or mRNA and the expression of EGFR protein and/or mRNA in a cell that is resistant to treatment with a protein tyrosine kinase inhibitor. The sequence of the human EGFR mRNA is shown in SEQ ID NO: 198. In yet further embodiments, the invention provides a method of inhibiting (e.g., by down-regulating) the expression of HER3, HER2 and EGFR mRNA and/or protein in a cell that is resistant to treatment with a protein tyrosine kinase inhibitor.

An oligomer as described herein typically binds to a target region of the human HER3 and/or the human HER2 and/or the human EGFR mRNA, and as such, comprises or consists of a region having a base sequence that is complementary or partially complementary to the base sequence of, e.g., SEQ ID NO 197, SEQ ID NO: 198 and/or SEQ ID NO: 199. In certain embodiments, the sequence of the oligomers described herein may optionally comprise 1, 2, 3, 4 or more base mismatches when compared to the sequence of the best-aligned target region of SEQ ID NOs: 197, 198 or 199.

In some embodiments, the oligomers described herein have sequences that are identical to a sequence selected from the group consisting of SEQ ID NOs: 200-227, 1-140 and 228-233 (see Table 1 herein below). In other embodiments, the oligomers have sequences that differ in one, two, or three bases when compared to a sequence selected from the group consisting of SEQ ID NOs: 200-227, 1-140 and 228-233. In some embodiments, the oligomers consist of or comprise 10-16 contiguous monomers. Examples of the sequences of oligomers consisting of 16 contiguous monomers are SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, and 140. Shorter sequences can be derived therefrom, e.g., the sequence of the shorter oligomer may be identically present in a region of an oligomer selected from those having base sequences of SEQ ID NOs: 200-227, 1-140 and 228-233. Longer oligomers may include a region having a sequence of at least 10 contiguous monomers that is identically present in SEQ ID NOs: 200-227, 1-140 and 228-233.

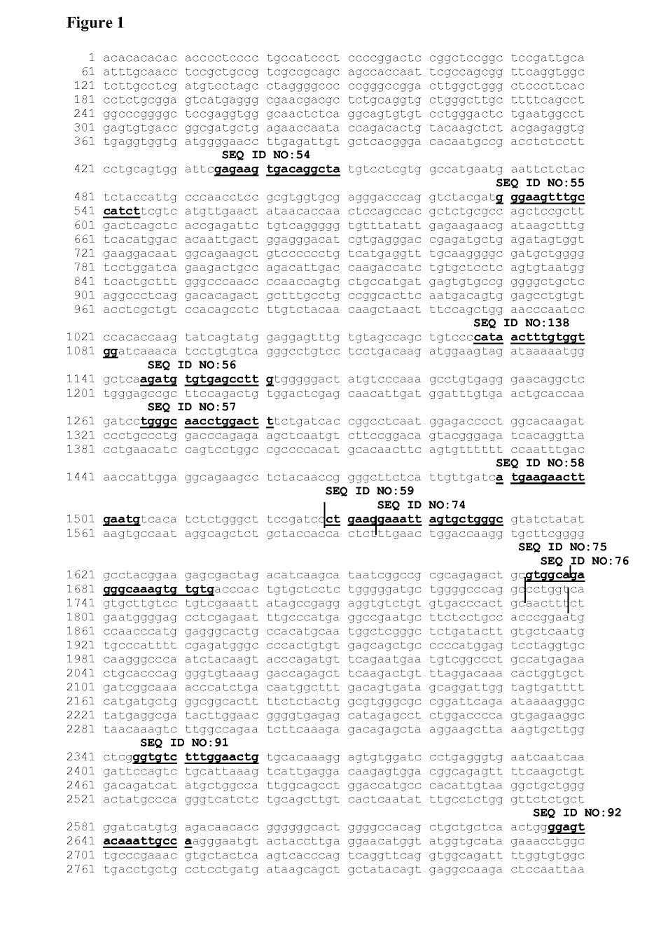

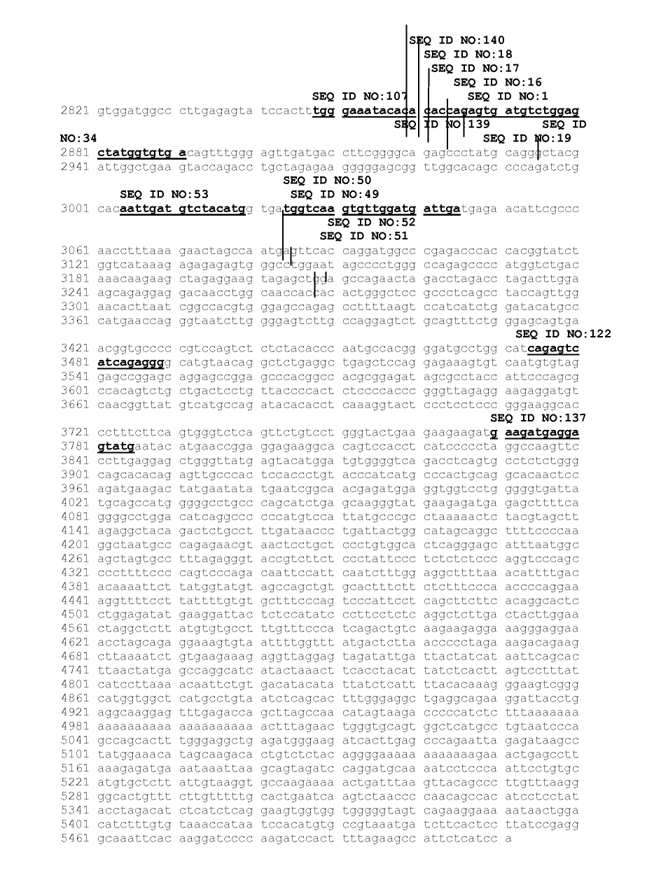

Further provided are target nucleic acids (e.g., DNA or mRNA encoding HER3), that contain target regions that are complementary or partially-complementary to one or more of the oligomers of SEQ ID NOs: 1-140, wherein the oligomers are capable of inhibiting expression (e.g., by down-regulation) of HER3 protein or mRNA. For example, target regions of human HER3 mRNA which are complementary to the antisense oligomers having sequences of SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, and 140 are shown in FIG. 1 (bold and underlined, with the corresponding oligomer SEQ ID NOs indicated above).

In various embodiments, the oligomers have the base sequences shown in SEQ ID NOs: 141-168. In certain embodiments, the oligomers are LNA oligomers, for example, those having the sequences of SEQ ID NOS: 169-196 and 234, in particular those having the base sequences of SEQ ID NOs: 169, 170, 173, 174, 180, 181, 183, 185, 187, 188, 189, 190, 191, 192 and 194. In various embodiments, the oligomers are LNA oligomers such as those having base sequences of SEQ ID NOs: 169, 170, 172, 174, 175, 176 and 179. In some embodiments, the oligomers or a region thereof consist of or comprise a base sequence as shown in SEQ ID NOs: 169, 180 or 234. In some embodiments, conjugates include an oligomer having a base sequence as shown in SEQ ID NOs: 169, 180 or 234.

In certain embodiments, the oligomer described herein may, suitably, comprise a region having a particular sequence, such as a sequence selected from SEQ ID NOs: 200-227, that is identically present in a shorter oligomer. Preferably, the region comprises 10-16 monomers. For example, the oligomers having the base sequences of SEQ ID NOs: 200-227 each comprise a region wherein the sequence of the region is identically present in shorter oligomers having sequences of SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, and 140, respectively. In some embodiments, oligomers which have fewer than 16 monomers, such as 10, 11, 12, 13, 14, or 15 monomers, have a region of at least 8, at least 9, at least 10, at least 11, at least 12, at least 13, at least 14 or 15, contiguous monomers of which the sequence is identically present in oligomers having sequences of SEQ ID NOS: 1, 16, 17, 18, 19, 34, 49, 50, 51, 52, 53, 54, 55, 56, 57, 58, 59, 74, 75, 76, 91, 92, 107, 122, 137, 138, 139, or 140. Hence, in various embodiments, the sequences of shorter oligomers are derived from the sequences of longer oligomers. In some embodiments, the sequences of oligomers having SEQ ID NOs disclosed herein, or the sequences of at least 10 contiguous monomers thereof, are identically present in longer oligomers. Typically an oligomer comprises a first region having a sequence that is identically present in SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, or 140, and if the oligomer is longer than the first region that is identically present in SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, or 140, the flanking regions of the oligomer have sequences that are complementary to the sequences flanking the target region of the target nucleic acid. Two such oligomers are SEQ ID NO: 1 and SEQ ID NO: 54.

In various embodiments, the oligomer comprises or consists of a sequence of monomers which is fully complementary (perfectly complementary) to a target region of a target nucleic acid which encodes a mammalian HER3.

However, in some embodiments, the sequence of the oligomer includes 1, 2, 3, or 4 (or more) mismatches as compared to the best-aligned target region of a HER3 target nucleic acid, and still sufficiently binds to the target region to effect inhibition of HER3 mRNA or protein expression. The destabilizing effect of mismatches on the Watson-Crick hydrogen-bonded duplex may, for example, be compensated by increased length of the oligomer and/or an increased number of nucleoside analogues, such as LNA monomers, present within the oligomer.

In various embodiments, the oligomer base sequence comprises no more than 3, such as no more than 2 mismatches compared to the base sequence of the best-aligned target region of, for example, a target nucleic acid which encodes a mammalian HER3.

The base sequences of the oligomers described herein or of a region thereof are preferably at least 80% identical to a sequence selected from the group consisting of SEQ ID NOS: 200-227, 1-140 and 228-233, such as at least 85%, at least 90%, at least 91%, at least 92%, at least 93%, at least 94%, at least 95%, at least 96%, at least 97%, at least 98%, at least 99%, even 100% identical.

The base sequences of the oligomers described herein or of a first region thereof are preferably at least 80% complementary to a sequence of a target region present in SEQ ID NOs: 197, 198 and/or 199 such as at least 85%, at least 90%, at least 91%, at least 92%, at least 93%, at least 94%, at least 95%, at least 96%, at least 97%, at least 98%, at least 99%, even 100% complementary.

In various embodiments, the sequence of the oligomer (or a first region thereof) is selected from the group consisting of SEQ ID NOs: 200-227, 1-140 and 228-233, or is selected from the group consisting of at least 10 contiguous monomers of SEQ ID NOs: 200-227, 1-140 and 228-233. In other embodiments, the sequence of the oligomer or a first region thereof optionally comprises 1, 2 or 3 base moieties that differ from those in oligomers having sequences of SEQ ID NOs: 200-227, 1-140 and 228-233, or the sequences of at least 10 contiguous monomers thereof, when optimally aligned with said selected sequence or region thereof.

In certain embodiments, the monomer region consists of 11, 12, 13, 14, 15, 16, 17, 18, 19, 20, 21, 22, 23, 24, 25, 26, 27, 28, or 29 contiguous monomers, such as between 10-15, 12-25, 12-22, such as between 12-18 monomers. Suitably, in various embodiments, the region is of the same length as the oligomer.

In some embodiments, the oligomer comprises additional monomers at the 5′ or 3′ ends, such as, independently, 1, 2, 3, 4 or 5 additional monomers 5′ end and/or 3′ end of the oligomer, which are non-complementary to the sequence of the target region. In various embodiments, the oligomer comprises a region that is complementary to the target, which is flanked 5′ and/or 3′ by additional monomers. In various embodiments, the 3′ end of the region is flanked by 1, 2 or 3 DNA or RNA monomers. 3′ DNA monomers are frequently used during solid state synthesis of oligomers. In various embodiments, which may be the same or different, the 5′ end of the oligomer is flanked by 1, 2 or 3 DNA or RNA monomers. In certain embodiments, the additional 5′ or 3′ monomers are nucleosides, such as DNA or RNA monomers. In various embodiments, the 5′ or 3′ monomers may represent region D as referred to in the context of gapmer oligomers herein.

In certain embodiments, the oligomer consists of, or comprises, contiguous monomers having a nucleobase sequence according to SEQ ID NO:200, or according to a region thereof.

In certain embodiments, the oligomer consists of, or comprises, contiguous monomers having a nucleobase sequence according to SEQ ID NO:201, or according to a region thereof.

In certain embodiments, the oligomer consists of, or comprises, contiguous monomers having a nucleobase sequence according to SEQ ID NO:202, or according to a region thereof.

In certain embodiments, the oligomer consists of, or comprises, contiguous monomers having a nucleobase sequence according to SEQ ID NO:203, or according to a region thereof.

In certain embodiments, the oligomer consists of, or comprises, contiguous monomers having a nucleobase sequence according to SEQ ID NO:204, or according to a region thereof.

In certain embodiments, the oligomer consists of, or comprises, contiguous monomers having a nucleobase sequence according to SEQ ID NO:205, or according to a region thereof.

In certain embodiments, the oligomer consists of, or comprises, contiguous monomers having a nucleobase sequence according to SEQ ID NO:206, or according to a region thereof.

In certain embodiments, the oligomer consists of, or comprises, contiguous monomers having a nucleobase sequence according to SEQ ID NO:207, or according to a region thereof.

In certain embodiments, the oligomer consists of, or comprises, contiguous monomers having a nucleobase sequence according to SEQ ID NO:208, or according to a region thereof.

In certain embodiments, the oligomer consists of, or comprises, contiguous monomers having a nucleobase sequence according to SEQ ID NO:209, or according to a region thereof.

In certain embodiments, the oligomer consists of, or comprises, contiguous monomers having a nucleobase sequence according to SEQ ID NO:210, or according to a region thereof.

In certain embodiments, the oligomer consists of, or comprises, contiguous monomers having a nucleobase sequence according to SEQ ID NO:211, or according to a region thereof.

In certain embodiments, the oligomer consists of, or comprises, contiguous monomers having a nucleobase sequence according to SEQ ID NO:212, or according to a region thereof.

In certain embodiments, the oligomer consists of, or comprises, contiguous monomers having a nucleobase sequence according to SEQ ID NO:213, or according to a region thereof.

In certain embodiments, the oligomer consists of, or comprises, contiguous monomers having a nucleobase sequence according to SEQ ID NO:214, or according to a region thereof.

In certain embodiments, the oligomer consists of, or comprises, contiguous monomers having a nucleobase sequence according to SEQ ID NO:215, or according to a region thereof.

In certain embodiments, the oligomer consists of, or comprises, contiguous monomers having a nucleobase sequence according to SEQ ID NO:216, or according to a region thereof.

In certain embodiments, the oligomer consists of, or comprises, contiguous monomers having a nucleobase sequence according to SEQ ID NO:217, or according to a region thereof.

In certain embodiments, the oligomer consists of, or comprises, contiguous monomers having a nucleobase sequence according to SEQ ID NO:218, or according to a region thereof.

In certain embodiments, the oligomer consists of, or comprises, contiguous monomers having a nucleobase sequence according to SEQ ID NO:219, or according to a region thereof.

In certain embodiments, the oligomer consists of, or comprises, contiguous monomers having a nucleobase sequence according to SEQ ID NO:220, or according to a region thereof.

In certain embodiments, the oligomer consists of, or comprises, contiguous monomers having a nucleobase sequence according to SEQ ID NO:221, or according to a region thereof.

In certain embodiments, the oligomer consists of, or comprises, contiguous monomers having a nucleobase sequence according to SEQ ID NO:222, or according to a region thereof.

In certain embodiments, the oligomer consists of, or comprises, contiguous monomers having a nucleobase sequence according to SEQ ID NO:223, or according to a region thereof.

In certain embodiments, the oligomer consists of, or comprises, contiguous monomers having a nucleobase sequence according to SEQ ID NO:224, or according to a region thereof.

In certain embodiments, the oligomer consists of, or comprises, contiguous monomers having a nucleobase sequence according to SEQ ID NO:225, or according to a region thereof.

In certain embodiments, the oligomer consists of, or comprises, contiguous monomers having a nucleobase sequence according to SEQ ID NO:226, or according to a region thereof.

In certain embodiments, the oligomer consists of, or comprises, contiguous monomers having a nucleobase sequence according to SEQ ID NO:227, or according to a region thereof.

Sequence alignments (such as those described above) can be used to identify regions of the nucleic acids encoding HER3 (or HER2 or EGFR) from human and one or more different mammalian species, such as monkey, mouse and/or rat, where there are sufficient stretches of nucleic acid identity between or among the species to allow the design of oligonucleotides which target (that is, which bind with sufficient specificity to inhibit expression of) both the human HER3 (or HER2 or EGFR) target nucleic acid and the corresponding nucleic acids present in the different mammalian species.

In some embodiments, such oligomers consist of or comprise regions of at least 10, such as at least 12, such as at least 14, such as at least 16, such as at least 18, such as 11, 12, 13, 14, 15, 16, 17 or 18 contiguous monomers which are 100% complementary in sequence to the sequence of the target regions of the nucleic acid encoding HER3 (or HER2 or EGFR) from humans and of the nucleic acid(s) encoding HER3 (or HER2 or EGFR) from a different mammalian species.

In some embodiments, the oligomer for use in the methods described herein comprises or consists of a region of contiguous monomers having a sequence that is at least 80%, such as at least 85%, such as at least 90%, such as at least 95%, such as at least 98% or 100% complementary to the sequence of the target regions of both the nucleic acid encoding human HER3 (or HER2 or EGFR) and a nucleic acid(s) encoding HER3 (or HER2 or EGFR) from a different mammalian species, such as the mouse nucleic acid encoding HER3 (or HER2 or EGFR). It is preferable that the contiguous nucleobase sequence of the oligomer is 100% complementary to the target region of the human HER3 (or HER2 or EGFR) mRNA.

In some embodiments, oligomers described herein bind to a target region of a HER3 target nucleic acid and down-regulate the expression of HER3 mRNA and/or protein. In various embodiments, oligomers described herein that bind to a target region of a HER3 nucleic acid have the sequences shown, for example, in SEQ ID NOs:169-196 and 234.

In some embodiments, a first region of a bispecific oligomer described herein binds to a target region of a HER 3 nucleic acid and a second region of the bispecific oligomer binds to a target region of a HER2 nucleic acid and said oligomer down-regulates the expression of HER3 and HER2. In various embodiments, the bispecific oligomer down-regulates the expression of HER 3 and HER2 to a different extent. In some embodiments, the first region and the second region of the oligomer are the same. In various embodiments, the first region and the second region of the oligomer overlap. In certain embodiments, the bispecific oligomers that bind to a target region of HER3 nucleic acid and a target region of HER2 nucleic acid have the sequences shown, for example, in SEQ ID NOs:177 and 178. In still other embodiments, a bispecific oligomer binds to a target region of HER3 nucleic acid and to a target region of EGFR nucleic acid and down-regulates the expression of HER3 and EGFR. In some embodiments, bispecific oligomers that bind to a target region of HER3 nucleic acid and to a target region of EGFR nucleic acid have the sequences shown, for example, in SEQ ID NOs: 171 and 173. In some embodiments, a first region of a bispecific oligomer described herein binds to a target region of HER 3 nucleic acid and a second region of the bispecific oligomer binds to a target region of EGFR nucleic acid and said oligomer down-regulates the expression of HER3 and EGFR. In various embodiments, the bispecific oligomer down-regulates the expression of HER3 and EGFR to a different extent. In some embodiments, the first region and the second region of the oligomer are the same. In various embodiments, the first region and the second region of the oligomer overlap. In yet further embodiments, trispecific oligomers described herein bind to a target region of HER3 nucleic acid, to a target region of HER2 nucleic acid and to a target region of EGFR nucleic acid and down-regulate the expression of all three genes. In some embodiments, trispecific oligomers that bind to HER3, HER2 and EGFR have the sequences shown, for example, in SEQ ID NOs: 169, 170, 172, 174-176 and 179. In some embodiments, a first region of a trispecific oligomer binds to a target region of HER 3 nucleic acid, a second region of the trispecific oligomer binds to a target region of EGFR nucleic acid, and a third region of the trispecific oligomer binds to a target region of HER2 nucleic acid, and said oligomers down-regulate the expression of HER3, HER2 and EGFR. In various embodiments, the trispecific oligomer down-regulates the expression of HER3, HER2 and EGFR to different extents. In some embodiments, the first, second and third regions of the oligomer are the same. In various embodiments, the first, second and third regions of the oligomer overlap. In various embodiments, bispecific or trispecific oligomers have 1, 2, 3, 4, 5 or more mismatches when compared to the best-aligned target regions of, e.g., target nucleic acids having sequences shown in SEQ ID NO: 197, 198 and/or 199.

5.6. Nucleosides and Nucleoside Analogues

In various embodiments, at least one of the monomers present in the oligomer is a nucleoside analogue that contains a modified base, such as a base selected from 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In various embodiments, at least one of the monomers present in the oligomer is a nucleoside analogue that contains a modified sugar.

In some embodiments, the linkage between at least 2 contiguous monomers of the oligomer is other than a phosphodiester linkage.

In certain embodiments, the oligomer includes at least one monomer that has a modified base, at least one monomer (which may be the same monomer) that has a modified sugar and at least one inter-monomer linkage that is non-naturally occurring.

Specific examples of nucleoside analogues useful in the oligomers described herein are described by e.g. Freier & Altmann; Nucl. Acid Res., 1997, 25, 4429-4443 and Uhlmann; Curr. Opinion in Drug Development, 2000, 3(2), 293-213, and in Scheme 1 (in which some nucleoside analogues are shown as nucleotides):

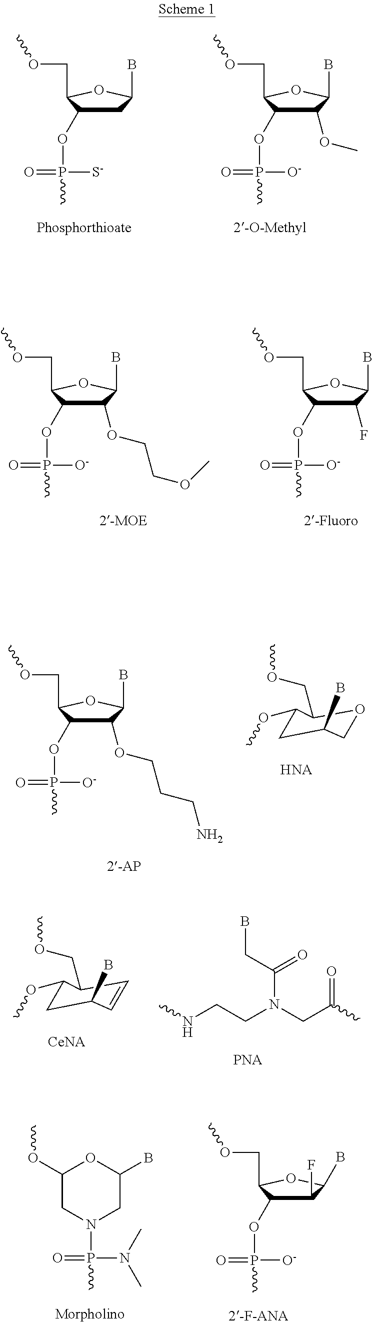

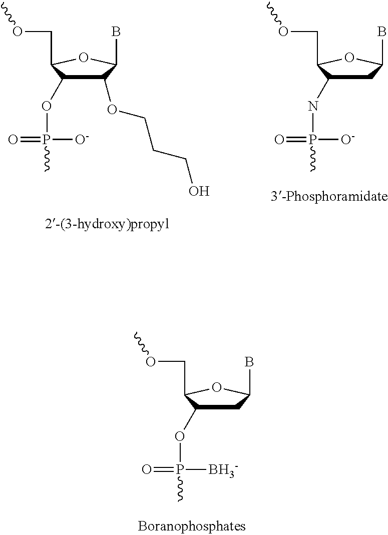

The oligomer may thus comprise or consist of a simple sequence of nucleosides—preferably DNA monomers, but also possibly RNA monomers, or a combination of nucleosides and one or more nucleoside analogues. In some embodiments, such nucleoside analogues suitably enhance the affinity of the oligomer for the target region of the target nucleic acid.

Examples of suitable and preferred nucleoside analogues are described in WO 2007/031091, incorporated herein by reference in its entirety, or are referenced therein.

In some embodiments, the nucleoside analogue comprises a sugar moiety modified to provide a 2′-substituent group, such as 2′-O-alkyl-ribose sugars, 2′-amino-deoxyribose sugars, and 2′-fluoro-deoxyribose sugars.

In some embodiments, the nucleoside analogue comprises a sugar in which a bridged structure, creating a bicyclic sugar (LNA), is present, which enhances binding affinity and may also provide some increased nuclease resistance. In various embodiments, the LNA monomer is selected from oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). In certain embodiments, the LNA monomers are beta-D-oxy-LNA. LNA monomers are further described below.

In various embodiments, incorporation of affinity-enhancing nucleoside analogues in the oligomer, such as LNA monomers or monomers containing 2′-substituted sugars, or incorporation of modified linkage groups provides increased nuclease resistance. In various embodiments, incorporation of such affinity-enhancing nucleoside analogues allows the size of the oligomer to be reduced, and also reduce the upper limit to the size of the oligomer before non-specific or aberrant binding takes place.

In certain embodiments, the oligomer comprises at least 2 nucleoside analogues. In some embodiments, the oligomer comprises from 3-8 nucleoside analogues, e.g. 6 or 7 nucleoside analogues. In preferred embodiments, at least one of the nucleoside analogues is a locked nucleic acid (LNA) monomer; for example at least 3 or at least 4, or at least 5, or at least 6, or at least 7, or 8 nucleoside analogues are LNA monomers. In some embodiments all the nucleosides analogues are LNA monomers.

It will be recognized that when referring to a preferred oligomer base sequence, in certain embodiments the oligomers comprise a corresponding nucleoside analogue, such as a corresponding LNA monomer or other corresponding nucleoside analogue, which raises the duplex stability (Tₘ) of the oligomer/target region duplex (i.e. affinity enhancing nucleoside analogues).

In various preferred embodiments, any mismatches (that is, noncomplementarities) between the base sequence of the oligomer and the base sequence of the target region, if present, are located other than in the regions of the oligomer that contain affinity-enhancing nucleoside analogues (e.g., regions A or C), such as within region B as referred to herein, and/or within region D as referred to herein, and/or in regions which are 5′ or 3′ to the region of the oligomer that is complementary to the target region.

In some embodiments the nucleoside analogues present within the oligomer (such as in regions A and C mentioned herein) are independently selected from, for example: monomers containing 2′-O-alkyl-ribose sugars, monomers containing 2′-amino-deoxyribose sugars, monomers containing 2′-fluoro-deoxyribose sugars, LNA monomers, monomers containing arabinose sugars (“ANA monomers”), monomers containing 2′-fluoro-arabinose sugars, monomers containing d-arabino-hexitol sugars (“HNA monomers”), intercalating monomers as defined in Christensen, Nucl. Acids. Res. 30: 4918-4925 (2002), hereby incorporated by reference, and monomers containing 2′MOE sugars. In certain embodiments, there is only one of the above types of nucleoside analogues present in the oligomer, or region thereof.

In certain embodiments, the nucleoside analogues contain 2′-O-methoxyethyl-ribose sugars (2′MOE), or 2′-fluoro-deoxyribose sugars or LNA sugars, and as such the oligonucleotide of the invention may comprise nucleoside analogues which are independently selected from these three types. In certain oligomer embodiments containing nucleoside analogues, at least one of said nucleoside analogues contains a 2′-MOE-ribose sugar, such as 2, 3, 4, 5, 6, 7, 8, 9 or 10 nucleoside analogues containing 2′-MOE-ribose sugars. In certain embodiments, at least one of said nucleoside analogues contains a 2′-fluoro-deoxyribose sugar, such as 2, 3, 4, 5, 6, 7, 8, 9 or 10 nucleoside analogues containing 2′-fluoro-deoxyribose sugars.

In various embodiments, the oligomer as described herein comprises at least one Locked Nucleic Acid (LNA) monomer, such as 1, 2, 3, 4, 5, 6, 7, or 8 LNA monomers, such as 3-7 or 4-8 LNA monomers, or 3, 4, 5, 6 or 7 LNA monomers. In various embodiments, all of the nucleoside analogues are LNA monomers. In some embodiments, the oligomer comprises both beta-D-oxy-LNA monomers, and one or more of the following LNA monomers: thio-LNA monomers, amino-LNA monomers, oxy-LNA monomers, and/or ENA monomers in either the beta-D or alpha-L configuration, or combinations thereof. In certain embodiments, the cytosine base moieties of all LNA monomers in the oligomer are 5-methylcytosines. In certain embodiments of the invention, the oligomer comprises both LNA and DNA monomers. Typically, the combined total of LNA and DNA monomers is 10-25, preferably 10-20, even more preferably 12-16. In certain embodiments of the invention, the oligomer or region thereof consists of at least one LNA monomer, and the remaining monomers are DNA monomers. In certain embodiments, the oligomer comprises only LNA monomers and nucleosides (such as RNA or DNA monomers, most preferably DNA monomers) optionally linked with modified linkage groups such as phosphorothioate.

In various embodiments, at least one of the nucleoside analogues present in the oligomer has a modified base selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

5.7. LNA

The term “LNA monomer” refers to a nucleoside analogue containing a bicyclic sugar (an “LNA sugar”). The terms “LNA oligonucleotide” and “LNA oligomer” refer to an oligomer containing one or more LNA monomers.

The LNA used in the oligonucleotide compounds of the invention preferably has the structure of the general formula I

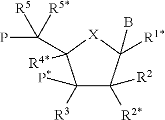

wherein X is selected from —O—, —S—, —N(R^{N*})—, —C(R⁶R^{6*})—;

B is selected from hydrogen, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;

P designates the radical position for an internucleoside linkage to a succeeding monomer, or a 5′-terminal group, such internucleoside linkage or 5′-terminal group optionally including the substituent R⁵ or equally applicable the substituent R^{5*};

P* designates an internucleoside linkage to a preceding monomer, or a 3′-terminal group;

R^{4*} and R^{2*} together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,
wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), and

each of the substituents R^{1*}, R², R³, R⁵, R^{5*}, R⁶ and R^{6*}, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof;

In certain embodiments, R^{5*} is selected from H, —CH₃, —CH₂—CH₃, —CH₂—O—CH₃, and —CH═CH₂.

In various embodiments, R^{4*} and R^{2*} together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂),

In further embodiments R^{4*} and R^{2*} together designate a biradical selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, —CH(CH₂—O—CH₃)—O—.

For all chiral centers, asymmetric groups may be found in either R or S orientation.

Preferably, the LNA monomer used in the oligomers described herein comprises at least one LNA monomer according to any of the formulas

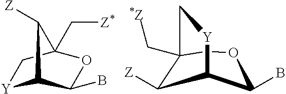

wherein Y is —O—, —O—CH₂—, —S—, —NH—, or N(R^{H}); Z and Z* are independently selected among an internucleoside linkage, a terminal group or a protecting group; B constitutes an unmodified base moiety or a modified base moiety that either occurs naturally in nucleic acids or does not occur naturally in nucleic acids, and R^{H} is selected from hydrogen and C₁₋₄-alkyl.

Specifically preferred LNA monomers are shown in Scheme 2:

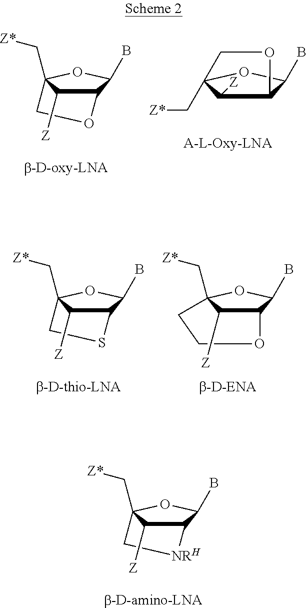

The term “thio-LNA” refers to an LNA monomer in which Y in the general formula above is selected from S or —CH₂—S—. Thio-LNA can be in either the beta-D or the alpha-L-configuration.

The term “amino-LNA” refers to an LNA monomer in which Y in the general formula above is selected from —N(H)—, N(R)—, CH₂—N(H)—, and —CH₂—N(R)— where R is selected from hydrogen and C₁₋₄-alkyl. Amino-LNA can be in either the beta-D or the alpha-L-configuration.

The term “oxy-LNA” refers to an LNA monomer in which Y in the general formula above represents —O— or —CH₂—O—. Oxy-LNA can be in either the beta-D or the alpha-L-configuration.

The term “ENA” refers to an LNA monomer in which Y in the general formula above is —CH₂—O— (where the oxygen atom of —CH₂—O— is attached to the 2′-position relative to the base B).

In a preferred embodiment the LNA monomer is selected from a beta-D-oxy-LNA monomer, an alpha-L-oxy-LNA monomer, a beta-D-amino-LNA monomer and a beta-D-thio-LNA monomer, in particular a beta-D-oxy-LNA monomer.

In the present context, the term “C1-4-alkyl” means a linear or branched saturated hydrocarbon chain wherein the chain has from one to four carbon atoms, such as methyl, ethyl, n-propyl, isopropyl, n-butyl, isobutyl, sec-butyl and tert-butyl.

5.8. RNAse H Recruitment

In some embodiments, an oligomer functions via non-RNase-mediated degradation of a target mRNA, such as by steric hindrance of translation, or other mechanisms; however, in various embodiments, oligomers described herein are capable of recruiting an endoribonuclease (RNase), such as RNase H.

Typically, the oligomer comprises a region of at least 6, such as at least 7 contiguous monomers, such as at least 8 or at least 9 contiguous monomers, including 7, 8, 9, 10, 11, 12, 13, 14, 15 or 16 contiguous monomers, which, when forming a duplex with the target region of the target RNA, is capable of recruiting RNase. The region of the oligomer which is capable of recruiting RNAse may be region B, as referred to in the context of a gapmer as described herein. In certain embodiments, the region of the oligomer which is capable of recruiting RNAse, such as region B, consists of 10, 11, 12, 13, 14, 15, 16, 17, 18, 19 or 20 monomers.

EP 1 222 309 provides in vitro methods for determining RNaseH activity, which may be used to determine the ability of the oligomers to recruit RNaseH. An oligomer is deemed capable of recruiting RNase H if, when contacted with the complementary target region of the RNA target, it has an initial rate, as measured in pmol/l/min, of at least 1%, such as at least 5%, such as at least 10% or less than 20% of an oligonucleotide having the same base sequence but containing only DNA monomers, with no 2′ substitutions, with phosphorothioate linkage groups between all monomers in the oligonucleotide, using the methodology provided by Example 91-95 of EP 1 222 309, incorporated herein by reference.

In various embodiments, an oligomer is deemed essentially incapable of recruiting RNaseH if, when contacted with the complementary target region of the RNA target, and RNaseH, the RNaseH initial rate, as measured in pmol/l/min, is less than 1%, such as less than 5%, such as less than 10% or less than 20% of the initial rate determined using an oligonucleotide having the same base sequence, but containing only DNA monomers, with no 2′ substitutions, with phosphorothioate linkage groups between all monomers in the oligonucleotide, using the methodology provided by Example 91-95 of EP 1 222 309.

In other embodiments, an oligomer is deemed capable of recruiting RNaseH if, when contacted with the complementary target region of the RNA target, and RNaseH, the RNaseH initial rate, as measured in pmol/l/min, is at least 20%, such as at least 40%, such as at least 60%, such as at least 80% of the initial rate determined using an oligonucleotide having the same base sequence, but containing only DNA monomers, with no 2′ substitutions, with phosphorothioate linkage groups between all monomers in the oligonucleotide, using the methodology provided by Example 91-95 of EP 1 222 309.

Typically, the region of the oligomer that forms a duplex with the complementary target region of the target RNA and is capable of recruiting RNase contains DNA monomers and LNA monomers and forms a DNA/RNA like duplex with the target region. The LNA monomers are preferably in the alpha-L configuration, particularly preferred being alpha-L-oxy LNA.

In various embodiments, the oligomer comprises both nucleosides and nucleoside analogues, and is in the form of a gapmer as defined above, a headmer or a mixmer.

A “headmer” is defined as an oligomer that comprises a first region and a second region that is contiguous thereto, with the 5′-most monomer of the second region linked to the 3′-most monomer of the first region. The first region comprises a contiguous stretch of non-RNase-recruiting nucleoside analogues, and the second region comprises a contiguous stretch (such as at least 7 contiguous monomers) of DNA monomers or nucleoside analogue monomers recognizable and cleavable by the RNAse.

A “tailmer” is defined as an oligomer that comprises a first region and a second region that is contiguous thereto, with the 5′-most monomer of the second region linked to the 3′-most monomer of the first region. The first region comprises a contiguous stretch (such as at least 7 such monomers) of DNA monomers or nucleoside analogue monomers recognizable and cleavable by the RNase, and the second region comprises a contiguous stretch of non-RNase recruiting nucleoside analogue monomers.

Other “chimeric” oligomers, called “mixmers”, consist of an alternating composition of (i) DNA monomers or nucleoside analogue monomers recognizable and cleavable by RNase, and (ii) non-RNase recruiting nucleoside analogue monomers.

In some embodiments, in addition to enhancing affinity of the oligomer for the target region, some nucleoside analogues also mediate RNase (e.g., RNase H) binding and cleavage. Since α-L-LNA monomers recruit RNase activity to a certain extent, in some embodiments, gap regions (e.g., region B as referred to herein below) of oligomers containing α-L-LNA monomers consist of fewer monomers recognizable and cleavable by the RNase, and more flexibility in the mixmer construction is introduced.

5.9. Conjugates

In the context of this disclosure, the term “conjugate” indicates a compound formed by the covalent attachment (“conjugation”) of an oligomer, as described herein, to one or more moieties that are not themselves nucleic acids or monomers (“conjugated moiety”). Examples of such conjugated moieties include macromolecular compounds such as proteins, fatty acid chains, sugar residues, glycoproteins, polymers, or combinations thereof. Typically, proteins may be antibodies for a target protein. Typical polymers may be polyethylene glycol. WO 2007/031091 provides suitable moieties and conjugates, which are hereby incorporated by reference.

Accordingly, provided herein are conjugates comprising an oligomer as herein described, and at least one conjugated moiety that is not a nucleic acid or monomer, covalently attached to said oligomer. Therefore, in certain embodiments, where the oligomer consists of contiguous monomers having a specified sequence of bases, as herein disclosed, the conjugate may also comprise at least one conjugated moiety that is covalently attached to said oligomer.

In certain embodiments, the oligomer is conjugated to a moiety that increases the cellular uptake of oligomeric compounds.

In various embodiments, conjugates may enhance the activity, cellular distribution or cellular uptake of the oligomers described herein. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

In certain embodiments, the oligomers are conjugated to active drug substances, for example, aspirin, ibuprofen, a sulfa drug, an antidiabetic, an antibacterial or an antibiotic.

In certain embodiments, the conjugated moiety is a sterol, such as cholesterol.

In various embodiments, the conjugated moiety comprises or consists of a positively charged polymer, such as a positively charged peptide of, for example 1-50, such as 2-20 such as 3-10 amino acid residues in length, and/or polyalkylene oxide such as polyethylene glycol (PEG) or polypropylene glycol—see WO 2008/034123, hereby incorporated by reference. Suitably, the positively charged polymer, such as a polyalkylene oxide may be attached to the oligomer via a linker such as the releasable inker described in WO 2008/034123.

5.10. Activated Oligomers

The term “activated oligomer,” as used herein, refers to an oligomer as described herein that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that in some embodiments is hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl.

In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers are functionalized at the 5′ end in order to allow covalent attachment of the conjugated moiety to the 5′ end of the oligomer. In other embodiments, oligomers can be functionalized at the 3′ end. In still other embodiments, oligomers can be functionalized along the backbone or on the heterocyclic base moiety. In yet other embodiments, oligomers can be functionalized at more than one position independently selected from the 5′ end, the 3′ end, the backbone and the base.

In some embodiments, activated oligomers as described herein are synthesized by incorporating during the synthesis one or more monomers that is covalently attached to a functional moiety. In other embodiments, activated oligomers are synthesized with monomers that have not been functionalized, and the oligomer is functionalized upon completion of synthesis.

In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH). In some embodiments, sulfhydryl-activated oligonucleotides are conjugated with polymer moieties such as polyethylene glycol or peptides (via formation of a disulfide bond).

Activated oligomers covalently linked to at least one functional moiety can be synthesized by any method known in the art, and in particular by methods disclosed in U.S. Patent Publication No. 2004/0235773, which is incorporated herein by reference in its entirety, and in Zhao et al. (2007) J. Controlled Release 119:143-152; and Zhao et al. (2005) Bioconjugate Chem. 16:758-766.

In still other embodiments, the oligomers described herein are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers have a functionalizing reagent coupled to a hydroxyl group on the backbone of the oligomer. In yet further embodiments, the oligomer is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers described herein have amine-containing functional moieties on the nucleobase, including on the N6 purine amino groups, on the exocyclic N2 of guanine, or on the N4 or 5 positions of cytosine. In some embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.).

5.11. Compositions

In various embodiments, the oligomer as described herein is used in pharmaceutical formulations and compositions. Suitably, such compositions comprise a pharmaceutically acceptable diluent, carrier, salt or adjuvant. WO2007/031091, which is hereby incorporated by reference, provides suitable and preferred pharmaceutically acceptable diluents, carriers and adjuvants. Suitable dosages, formulations, administration routes, compositions, dosage forms, combinations with other therapeutic agents, pro-drug formulations are also provided in WO2007/031091, which are also hereby incorporated by reference. Details on techniques for formulation and administration also may be found in the latest edition of “REMINGTON'S PHARMACEUTICAL SCIENCES” (Maack Publishing Co, Easton Pa.).

In some embodiments, an oligomer described herein is covalently linked to a conjugated moiety to aid in delivery of the oligomer across cell membranes. An example of a conjugated moiety that aids in delivery of the oligomer across cell membranes is a lipophilic moiety, such as cholesterol. In various embodiments, an oligomer as described herein is formulated with lipid formulations that form liposomes, such as Lipofectamine 2000 or Lipofectamine RNAiMAX, both of which are commercially available from Invitrogen. In some embodiments, the oligomers described herein are formulated with a mixture of one or more lipid-like non-naturally occurring small molecules (“lipidoids”). Libraries of lipidoids can be synthesized by conventional synthetic chemistry methods and various amounts and combinations of lipidoids can be assayed in order to develop a vehicle for effective delivery of an oligomer of a particular size to the targeted tissue by the chosen route of administration. Suitable lipidoid libraries and compositions can be found, for example in Akinc et al. (2008) Nature Biotechnol., available at http://www.nature.com/nbt/journal/vaop/ncurrent/abs/nbt1402.html, which is incorporated by reference herein.

As used herein, the term “pharmaceutically acceptable salts” refers to salts that retain the desired biological activity of the herein identified compounds and exhibit acceptable levels of undesired toxic effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N′-dibenzylethylene-diamine, D-glucosamine, tetraethylammonium, or ethylenediamine; or (c) combinations of (a) and (b); e.g., a zinc tannate salt or the like.

The amount of the at least one oligomer that is effective for the treatment or prevention of a disease that is resistant to treatment with a PTK inhibitor can be determined by standard clinical techniques. Generally the dosage ranges can be estimated based on EC₅₀ found to be effective in in vitro and in vivo animal models. The precise doses to be employed will also depend on, e.g., the routes of administration and the seriousness of the disease, and can be decided according to the judgment of a practitioner and/or each patient's circumstances. In other examples thereof, variations will necessarily occur depending upon, inter alia, the weight and physical condition (e.g., hepatic and renal function) of the patient being treated, the affliction to be treated, the severity of the symptoms, the frequency of the dosage interval, and the presence of any deleterious side-effects.

In various embodiments, the dosage of an oligomer is from about 0.01 μg to about 1 g per kg of body weight, and may be given once or more daily, weekly, monthly or yearly, or even once every 2 to 10 years or by continuous infusion for hours up to several months. In certain embodiments, repetition rates for dosing can be estimated based on measured residence times and concentrations of the active agent in bodily fluids or tissues. Following successful treatment, the patient can undergo maintenance therapy with the HER3-targeted therapy to prevent the recurrence of the disease state.

5.12. Combination with Other Antisense oligomers and Chemotherapeutic Agents

In some embodiments, oligomers described herein are targeted to HER3, HER2 and/or EGFR nucleic acids. Thus, in some embodiments, the invention relates to methods of treating a disease that is resistant to treatment with a PTK inhibitor by administering more than one oligomer to target two or even all three target nucleic acids. In various embodiments, an oligomer which targets HER3 is administered with a second oligomer which targets either EGFR or HER2. In various other embodiments, an oligomer which targets HER3 is administered with a second oligomer which targets HER2 and a third oligomer that targets EGFR. In the methods described herein, such oligomers can be administered concurrently, or sequentially.

In various embodiments the invention relates to methods of treating a PTK inhibitor-resistant disease by administering a pharmaceutical composition that comprises an oligomer targeted to HER3, and a further therapeutic agent which targets and down-regulates HER2 expression, such as an antisense oligomer which targets HER2 mRNA.

In other embodiments, which may be the same or different, the invention relates to a method of treating a PTK inhibitor-resistant disease by administering a pharmaceutical composition comprising an oligomer targeted to HER3, and a further therapeutic agent which targets and down-regulates EGFR expression, such as an antisense oligomer which target EGFR mRNA.

In some embodiments, oligomers that target HER2 and/or EGFR mRNA (or conjugates thereof), have the same designs (e.g., gapmers, headmers, tailmers) as oligomers that target HER3. In various embodiments, oligomers that target HER2 and/or EGFR mRNA (or conjugates thereof), have different designs from oligomers that target HER3.

In certain embodiments, the invention relates to a method of treating a PTK inhibitor-resistant disease by administering one or more oligomers as described herein and one or more additional chemotherapeutic agents, including but not limited to, alkylating agents, anti-metabolites, epipodophyllotoxins, anthracyclines, vinca alkaloids, plant alkaloids and terpenoids, monoclonal antibodies, taxanes, topoisomerase inhibitors, and platinum compounds.

5.13. Kits

The invention also provides methods of treating a disease that is resistant to treatment with a protein tyrosine kinase inhibitor using a kit comprising a first component and a second component. In various embodiments, said first component comprises an oligomer as described herein that is capable of inhibiting (e.g., by down-regulating) expression of HER3, or a conjugate and/or pharmaceutical composition thereof. In other embodiments, the second component comprises a second active ingredient. In some embodiments, the second component is a therapeutic agent that is an oligonucleotide as described herein. In other embodiments, the therapeutic agent is other than an oligonucleotide (e.g., a small molecule therapeutic agent such as taxol). In some embodiments, kits described herein are used in methods of treating a hyperproliferative disorder, such as cancer which is resistant to treatment with a PTK inhibitor, which comprises administering to a patient in need thereof an effective amount of a first component and a second component of the kit. In various embodiments, the first and second components are administered simultaneously. In other embodiments, the first and second components are administered sequentially and in any order.

In some embodiments, the kit comprises a first component that comprises an oligomer that is capable of inhibiting (e.g., by down-regulating) expression of HER3, or a conjugate and/or pharmaceutical composition thereof, and a second component that is an antisense oligonucleotide capable of inhibiting (e.g., by down-regulating) the expression of HER2 and/or EGFR expression as described herein, or a conjugate and/or pharmaceutical composition thereof

### 6. EXAMPLES

### 6.1. Example 1

### Monomer Synthesis

The LNA monomer building blocks and derivatives thereof were prepared according to published procedures. See WO07/031,081 and the references cited therein.

### 6.2. Example 2

### Oligonucleotide Synthesis

Oligonucleotides were synthesized according to the method described in WO07/031,081. Table 1 shows examples of antisense oligonucleotide motifs of the invention.

### 6.3. Example 3

### Design of the Oligonucleotides

In accordance with the invention, a series of oligonucleotides were designed to target different regions of human EGFR (GenBank Accession number NM_{—}005228, SEQ ID NO: 198) and human HER2 (GenBank Accession number NM_{—}004448, SEQ ID NO: 199) in addition to human HER3 (GenBank Accession number NM_{—}001982, SEQ ID NO: 197).

Of the sequences shown in Table 1, below, SEQ ID NOs: 1-50, 53, 139 and 140 were designed to target human EGFR and human HER2 in addition to human HER3. The percentage of sequence homology with HER3, EGFR and HER2 is indicated. The sequences of the oligomers contain 0-2 mismatches when compared to the sequences of the best-aligned target regions of EGFR, and 1-2 mismatches when compared to the sequences of the best-aligned target regions of HER2.

TABLE 1
Antisense Oligonucleotide Sequences

| | | Length | | Compl | Compl |
|---|---|---|---|---|---|
| SEQ ID NO | Sequence (5′-3′) | (bases) | Target site HER3 | EGFR | HER2 |
| SEQ ID NO: 1 | GCTCCAGACATCACTC | 16 | 2866-2881 | 100% | 87.5% |
| SEQ ID NO: 2 | GCTCCAGACATCACT | 15 | | | |
| SEQ ID NO: 3 | CTCCAGACATCACTC | 15 | | | |
| SEQ ID NO: 4 | GCTCCAGACATCAC | 14 | | | |
| SEQ ID NO: 5 | CTCCAGACATCACT | 14 | | | |
| SEQ ID NO: 6 | TCCAGACATCACTC | 14 | | | |
| SEQ ID NO: 7 | GCTCCAGACATCA | 13 | | | |
| SEQ ID NO: 8 | CTCCAGACATCAC | 13 | | | |
| SEQ ID NO: 9 | TCCAGACATCACT | 13 | | | |
| SEQ ID NO: 10 | CCAGACATCACTC | 13 | | | |
| SEQ ID NO: 11 | GCTCCAGACATC | 12 | | | |
| SEQ ID NO: 12 | CTCCAGACATCA | 12 | | | |
| SEQ ID NO: 13 | TCCAGACATCAC | 12 | | | |
| SEQ ID NO: 14 | CCAGACATCACT | 12 | | | |
| SEQ ID NO: 15 | CAGACATCACTC | 12 | | | |
| SEQ ID NO: 16 | CTCCAGACATCACTCT | 16 | 2865-2880 | 100% | 93.8% |
| SEQ ID NO: 17 | CAGACATCACTCTGGT | 16 | 2862-2877 | 100% | 93.8% |
| SEQ ID NO: 18 | AGACATCACTCTGGTG | 16 | 2861-2876 | 100% | 93.8% |
| SEQ ID NO: 19 | ATAGCTCCAGACATCA | 16 | 2869-2884 | 93.8% | 87.5% |
| SEQ ID NO: 20 | ATAGCTCCAGACATC | 15 | | | |
| SEQ ID NO: 21 | TAGCTCCAGACATCA | 15 | | | |
| SEQ ID NO: 22 | ATAGCTCCAGACAT | 14 | | | |
| SEQ ID NO: 23 | TAGCTCCAGACATC | 14 | | | |
| SEQ ID NO: 24 | AGCTCCAGACATCA | 14 | | | |
| SEQ ID NO: 25 | ATAGCTCCAGACA | 13 | | | |
| SEQ ID NO: 26 | TAGCTCCAGACAT | 13 | | | |
| SEQ ID NO: 27 | AGCTCCAGACATC | 13 | | | |
| SEQ ID NO: 28 | GCTCCAGACATCA | 13 | | | |
| SEQ ID NO: 29 | ATAGCTCCAGAC | 12 | | | |
| SEQ ID NO: 30 | TAGCTCCAGACA | 12 | | | |
| SEQ ID NO: 31 | AGCTCCAGACAT | 12 | | | |
| SEQ ID NO: 32 | GCTCCAGACATC | 12 | | | |
| SEQ ID NO: 33 | CTCCAGACATCA | 12 | | | |
| SEQ ID NO: 34 | TCACACCATAGCTCCA | 16 | 2876-2891 | 87.5% | 93.8% |
| SEQ ID NO: 35 | TCACACCATAGCTCC | 15 | | | |
| SEQ ID NO: 36 | CACACCATAGCTCCA | 15 | | | |
| SEQ ID NO: 37 | TCACACCATAGCTC | 14 | | | |
| SEQ ID NO: 38 | CACACCATAGCTCC | 14 | | | |
| SEQ ID NO: 39 | ACACCATAGCTCCA | 14 | | | |
| SEQ ID NO: 40 | TCACACCATAGCT | 13 | | | |
| SEQ ID NO: 41 | CACACCATAGCTC | 13 | | | |
| SEQ ID NO: 42 | ACACCATAGCTCC | 13 | | | |
| SEQ ID NO: 43 | CACCATAGCTCCA | 13 | | | |
| SEQ ID NO: 44 | TCACACCATAGC | 12 | | | |
| SEQ ID NO: 45 | CACACCATAGCT | 12 | | | |
| SEQ ID NO: 46 | ACACCATAGCTC | 12 | | | |
| SEQ ID NO: 47 | CACCATAGCTCC | 12 | | | |
| SEQ ID NO: 48 | ACCATAGCTCCA | 12 | | | |
| SEQ ID NO: 49 | CATCCAACACTTGACC | 16 | 3025-3040 | 93.8% | 93.8% |
| SEQ ID NO: 50 | ATCCAACACTTGACCA | 16 | 3024-3039 | 93.8% | 93.8% |
| SEQ ID NO: 51 | CAATCATCCAACACTT | 16 | 3029-3044 | 87.5% | 93.8% |
| SEQ ID NO: 52 | TCAATCATCCAACACT | 16 | 3030-3045 | 87.5% | 93.8% |
| SEQ ID NO: 53 | CATGTAGACATCAATT | 16 | 3004-3019 | 87.5% | 93.8% |
| SEQ ID NO: 54 | TAGCCTGTCACTTCTC | 16 | 435-450 | 68.8% | 75% |
| SEQ ID NO: 228 | TAGCCTGTCACTTCT | 15 | | | |
| SEQ ID NO: 229 | AGCCTGTCACTTCTC | 15 | | | |
| SEQ ID NO: 230 | TAGCCTGTCACTTC | 14 | | | |
| SEQ ID NO: 231 | AGCCTGTCACTTCT | 14 | | | |
| SEQ ID NO: 232 | TAGCCTGTCACTT | 13 | | | |
| SEQ ID NO: 233 | TAGCCTGTCACT | 12 | | | |
| SEQ ID NO: 55 | AGATGGCAAACTTCCC | 16 | 530-545 | 68.8% | 68.8% |
| SEQ ID NO: 56 | CAAGGCTCACACATCT | 16 | 1146-1161 | 75% | 68.8% |
| SEQ ID NO: 57 | AAGTCCAGGTTGCCCA | 16 | 1266-1281 | 75% | 75% |
| SEQ ID NO: 58 | CATTCAAGTTCTTCAT | 16 | 1490-1505 | 75% | 68.8% |
| SEQ ID NO: 59 | CACTAATTTCCTTCAG | 16 | 1529-1544 | 81.3% | 68.8% |
| SEQ ID NO: 60 | CACTAATTTCCTTCA | 15 | | | |
| SEQ ID NO: 61 | ACTAATTTCCTTCAG | 15 | | | |
| SEQ ID NO: 62 | CACTAATTTCCTTC | 14 | | | |
| SEQ ID NO: 63 | ACTAATTTCCTTCA | 14 | | | |
| SEQ ID NO: 64 | CTAATTTCCTTCAG | 14 | | | |
| SEQ ID NO: 65 | CACTAATTTCCTT | 13 | | | |
| SEQ ID NO: 66 | ACTAATTTCCTTC | 13 | | | |
| SEQ ID NO: 67 | CTAATTTCCTTCA | 13 | | | |
| SEQ ID NO: 68 | TAATTTCCTTCAG | 13 | | | |
| SEQ ID NO: 69 | CACTAATTTCCT | 12 | | | |
| SEQ ID NO: 70 | ACTAATTTCCTT | 12 | | | |
| SEQ ID NO: 71 | CTAATTTCCTTC | 12 | | | |
| SEQ ID NO: 72 | TAATTTCCTTCA | 12 | | | |
| SEQ ID NO: 73 | AATTTCCTTCAG | 12 | | | |
| SEQ ID NO: 74 | GCCCAGCACTAATTTC | 16 | 1535-1550 | 75% | 68.8% |
| SEQ ID NO: 75 | CTTTGCCCTCTGCCAC | 16 | 1673-1688 | 75% | 75% |
| SEQ ID NO: 76 | CACACACTTTGCCCTC | 16 | 1679-1694 | 68.8% | 75% |
| SEQ ID NO: 77 | CACACACTTTGCCCT | 15 | | | |
| SEQ ID NO: 78 | ACACACTTTGCCCTC | 15 | | | |
| SEQ ID NO: 79 | CACACACTTTGCCC | 14 | | | |
| SEQ ID NO: 80 | ACACACTTTGCCCT | 14 | | | |
| SEQ ID NO: 81 | CACACTTTGCCCTC | 14 | | | |
| SEQ ID NO: 82 | CACACACTTTGCC | 13 | | | |
| SEQ ID NO: 83 | ACACACTTTGCCC | 13 | | | |
| SEQ ID NO: 84 | CACACTTTGCCCT | 13 | | | |
| SEQ ID NO: 85 | ACACTTTGCCCTC | 13 | | | |
| SEQ ID NO: 86 | CACACACTTTGC | 12 | | | |
| SEQ ID NO: 87 | ACACACTTTGCC | 12 | | | |
| SEQ ID NO: 88 | CACACTTTGCCC | 12 | | | |
| SEQ ID NO: 89 | ACACTTTGCCCT | 12 | | | |
| SEQ ID NO: 90 | CACTTTGCCCTC | 12 | | | |
| SEQ ID NO: 91 | CAGTTCCAAAGACACC | 16 | 2345-2360 | 75% | 68.8% |
| SEQ ID NO: 92 | TGGCAATTTGTACTCC | 16 | 2636-2651 | 75% | 68.8% |
| SEQ ID NO: 93 | TGGCAATTTGTACTC | 15 | | | |
| SEQ ID NO: 94 | GGCAATTTGTACTCC | 15 | | | |
| SEQ ID NO: 95 | TGGCAATTTGTACT | 14 | | | |
| SEQ ID NO: 96 | GGCAATTTGTACTC | 14 | | | |
| SEQ ID NO: 97 | GCAATTTGTACTCC | 14 | | | |
| SEQ ID NO: 98 | TGGCAATTTGTAC | 13 | | | |
| SEQ ID NO: 99 | GGCAATTTGTACT | 13 | | | |
| SEQ ID NO: 100 | GCAATTTGTACTC | 13 | | | |
| SEQ ID NO: 101 | CAATTTGTACTCC | 13 | | | |
| SEQ ID NO: 102 | TGGCAATTTGTA | 12 | | | |
| SEQ ID NO: 103 | GGCAATTTGTAC | 12 | | | |
| SEQ ID NO: 104 | GCAATTTGTACT | 12 | | | |
| SEQ ID NO: 105 | CAATTTGTACTC | 12 | | | |
| SEQ ID NO: 106 | AATTTGTACTCC | 12 | | | |
| SEQ ID NO: 107 | GTGTGTGTATTTCCCA | 16 | 2848-2863 | 75% | 68.8% |
| SEQ ID NO: 108 | GTGTGTGTATTTCCC | 15 | | | |
| SEQ ID NO: 109 | TGTGTGTATTTCCCA | 15 | | | |
| SEQ ID NO: 110 | GTGTGTGTATTTCC | 14 | | | |
| SEQ ID NO: 111 | TGTGTGTATTTCCC | 14 | | | |
| SEQ ID NO: 112 | GTGTGTATTTCCCA | 14 | | | |
| SEQ ID NO: 113 | GTGTGTGTATTTC | 13 | | | |
| SEQ ID NO: 114 | TGTGTGTATTTCC | 13 | | | |
| SEQ ID NO: 115 | GTGTGTATTTCCC | 13 | | | |
| SEQ ID NO: 116 | TGTGTATTTCCCA | 13 | | | |
| SEQ ID NO: 117 | GTGTGTGTATTT | 12 | | | |
| SEQ ID NO: 118 | TGTGTGTATTTC | 12 | | | |
| SEQ ID NO: 119 | GTGTGTATTTCC | 12 | | | |
| SEQ ID NO: 120 | TGTGTATTTCCC | 12 | | | |
| SEQ ID NO: 121 | GTGTATTTCCCA | 12 | | | |
| SEQ ID NO: 122 | CCCTCTGATGACTCTG | 16 | 3474-3489 | 68.8% | 68.8% |
| SEQ ID NO: 123 | CCCTCTGATGACTCT | 15 | | | |
| SEQ ID NO: 124 | CCTCTGATGACTCTG | 15 | | | |
| SEQ ID NO: 125 | CCCTCTGATGACTC | 14 | | | |
| SEQ ID NO: 126 | CCTCTGATGACTCT | 14 | | | |
| SEQ ID NO: 127 | CTCTGATGACTCTG | 14 | | | |
| SEQ ID NO: 128 | CCCTCTGATGACT | 13 | | | |
| SEQ ID NO: 129 | CCTCTGATGACTC | 13 | | | |
| SEQ ID NO: 130 | CTCTGATGACTCT | 13 | | | |
| SEQ ID NO: 131 | TCTGATGACTCTG | 13 | | | |
| SEQ ID NO: 132 | CCCTCTGATGAC | 12 | | | |
| SEQ ID NO: 133 | CCTCTGATGACT | 12 | | | |
| SEQ ID NO: 134 | CTCTGATGACTC | 12 | | | |
| SEQ ID NO: 135 | TCTGATGACTCT | 12 | | | |
| SEQ ID NO: 136 | CTGATGACTCTG | 12 | | | |
| SEQ ID NO: 137 | CATACTCCTCATCTTC | 16 | 3770-3785 | 81.3% | 81.3% |
| SEQ ID NO: 138 | CCACCACAAAGTTATG | 16 | 1067-1082 | 81.3% | 68.8% |
| SEQ ID NO: 139 | CATCACTCTGGTGTGT | 16 | 2858-2873 | 93.8% | 93.8% |
| SEQ ID NO: 140 | GACATCACTCTGGTGT | 16 | 2860-2875 | 93.8% | 87.5% |

In Table 2, bold letters represent shorter sequences shown in Table 1.

TABLE 2
HERS 24 mer Sequences

| 16 mer SEQ IDs | Corresponding 24 mer sequence comprising 16 mer | 24 mer SEQ ID |
|---|---|---|
| SEQ ID NO: 1 | catagctccagacatcactctggt | SEQ ID NO: 200 |
| SEQ ID NO: 16 | atagctccagacatcactctggtg | SEQ ID NO: 201 |
| SEQ ID NO: 17 | gctccagacatcactctggtgtgt | SEQ ID NO: 202 |
| SEQ ID NO: 18 | ctccagacatcactctggtgtgtg | SEQ ID NO: 203 |
| SEQ ID NO: 19 | caccatagctccagacatcactct | SEQ ID NO: 204 |
| SEQ ID NO: 34 | actgtcacaccatagctccagaca | SEQ ID NO: 205 |
| SEQ ID NO: 49 | caatcatccaacacttgaccatca | SEQ ID NO: 206 |
| SEQ ID NO: 50 | aatcatccaacacttgaccatcac | SEQ ID NO: 207 |
| SEQ ID NO: 51 | tcatcaatcatccaacacttgacc | SEQ ID NO: 208 |
| SEQ ID NO: 52 | ctcatcaatcatccaacacttgac | SEQ ID NO: 209 |
| SEQ ID NO: 53 | tcaccatgtagacatcaattgtgc | SEQ ID NO: 210 |
| SEQ ID NO: 54 | gacatagcctgtcacttctcgaat | SEQ ID NO: 211 |
| SEQ ID NO: 55 | acgaagatggcaaacttcccatcg | SEQ ID NO: 212 |
| SEQ ID NO: 56 | cccacaaggctcacacatcttgag | SEQ ID NO: 213 |
| SEQ ID NO: 57 | cagaaagtccaggttgcccaggat | SEQ ID NO: 214 |
| SEQ ID NO: 58 | gtgacattcaagttcttcatgatc | SEQ ID NO: 215 |
| SEQ ID NO: 59 | ccagcactaatttccttcagggat | SEQ ID NO: 216 |
| SEQ ID NO: 74 | atacgcccagcactaatttccttc | SEQ ID NO: 217 |
| SEQ ID NO: 75 | cacactttgccctctgccacgcag | SEQ ID NO: 218 |
| SEQ ID NO: 76 | gggtcacacactttgccctctgcc | SEQ ID NO: 219 |
| SEQ ID NO: 91 | tgcacagttccaaagacacccgag | SEQ ID NO: 220 |
| SEQ ID NO: 92 | cccttggcaatttgtactccccag | SEQ ID NO: 221 |
| SEQ ID NO: 107 | tctggtgtgtgtatttcccaaagt | SEQ ID NO: 222 |
| SEQ ID NO: 122 | atgcccctctgatgactctgatgc | SEQ ID NO: 223 |
| SEQ ID NO: 137 | tattcatactcctcatcttcatct | SEQ ID NO: 224 |
| SEQ ID NO: 138 | tgatccaccacaaagttatgggga | SEQ ID NO: 225 |
| SEQ ID NO: 139 | cagacatcactctggtgtgtgtat | SEQ ID NO: 226 |
| SEQ ID NO: 140 | tccagacatcactctggtgtgtgt | SEQ ID NO: 227 |

In SEQ ID NOs: 141-168 shown in Table 3, uppercase letters indicate nucleoside analogue monomers and the subscript “s” represents a phosphorothioate linkage. Lowercase letters represent DNA monomers. The absence of “s” between monomers (if any) indicates a phosphodiester linkage.

TABLE 3
Oligonucleotide gapmers

| SEQ ID NO | Sequence (5′-3′) |
|---|---|
| SEQ ID NO: 141 | G_{S}C_{S}T_{S}c_{S}c_{S}a_{S}g_{S}a_{S}c_{S}a_{S}t_{S}c_{S}a_{S}C_{S}T_{S}C |
| SEQ ID NO: 142 | C_{S}T_{S}C_{S}c_{S}a_{S}g_{S}a_{S}c_{S}a_{S}t_{S}c_{S}a_{S}c_{S}T_{S}C_{S}T |
| SEQ ID NO: 143 | C_{S}A_{S}G_{S}a_{S}c_{S}a_{S}t_{S}c_{S}a_{S}c_{S}t_{S}c_{S}t_{S}G_{S}G_{S}T |
| SEQ ID NO: 144 | A_{S}G_{S}A_{S}c_{S}a_{S}t_{S}c_{S}a_{S}c_{S}t_{S}c_{S}t_{S}g_{S}G_{S}T_{S}G |
| SEQ ID NO: 145 | A_{S}T_{S}A_{S}g_{S}c_{S}t_{S}c_{S}c_{S}a_{S}g_{S}a_{S}c_{S}a_{S}T_{S}C_{S}A |
| SEQ ID NO: 146 | T_{S}C_{S}A_{S}c_{S}a_{S}c_{S}c_{S}a_{S}t_{S}a_{S}g_{S}c_{S}t_{S}C_{S}C_{S}A |
| SEQ ID NO: 147 | C_{S}A_{S}T_{S}c_{S}c_{S}a_{S}a_{S}c_{S}a_{S}c_{S}t_{S}t_{S}g_{S}A_{S}C_{S}C |
| SEQ ID NO: 148 | A_{S}T_{S}C_{S}c_{S}a_{S}a_{S}c_{S}a_{S}c_{S}t_{S}t_{S}g_{S}a_{S}C_{S}C_{S}A |
| SEQ ID NO: 149 | C_{S}A_{S}A_{S}t_{S}c_{S}a_{S}t_{S}c_{S}c_{S}a_{S}a_{S}c_{S}a_{S}C_{S}T_{S}T |
| SEQ ID NO: 150 | T_{S}C_{S}A_{S}a_{S}t_{S}c_{S}a_{S}t_{S}c_{S}c_{S}a_{S}a_{S}c_{S}A_{S}C_{S}T |
| SEQ ID NO: 151 | C_{S}A_{S}T_{S}g_{S}t_{S}a_{S}g_{S}a_{S}c_{S}a_{S}t_{S}c_{S}a_{S}A_{S}T_{S}T |
| SEQ ID NO: 152 | T_{S}A_{S}G_{S}c_{S}c_{S}t_{S}g_{S}t_{S}c_{S}a_{S}c_{S}t_{S}t_{S}C_{S}T_{S}C |
| SEQ ID NO: 153 | A_{S}G_{S}A_{S}t_{S}g_{S}g_{S}c_{S}a_{S}a_{S}a_{S}c_{S}t_{S}t_{S}C_{S}C_{S}C |
| SEQ ID NO: 154 | C_{S}A_{S}A_{S}g_{S}g_{S}c_{S}t_{S}c_{S}a_{S}c_{S}a_{S}c_{S}a_{S}T_{S}C_{S}T |
| SEQ ID NO: 155 | A_{S}A_{S}G_{S}t_{S}c_{S}c_{S}a_{S}g_{S}g_{S}t_{S}t_{S}g_{S}c_{S}C_{S}C_{S}A |
| SEQ ID NO: 156 | C_{S}A_{S}T_{S}t_{S}c_{S}a_{S}a_{S}g_{S}t_{S}t_{S}c_{S}t_{S}t_{S}C_{S}A_{S}T |
| SEQ ID NO: 157 | C_{S}A_{S}C_{S}t_{S}a_{S}a_{S}t_{S}t_{S}t_{S}c_{S}c_{S}t_{S}t_{S}C_{S}A_{S}G |
| SEQ ID NO: 158 | G_{S}C_{S}C_{S}c_{S}a_{S}g_{S}c_{S}a_{S}c_{S}t_{S}a_{S}a_{S}t_{S}T_{S}T_{S}C |
| SEQ ID NO: 159 | C_{S}T_{S}T_{S}t_{S}g_{S}c_{S}c_{S}c_{S}t_{S}c_{S}t_{S}g_{S}c_{S}C_{S}A_{S}C |
| SEQ ID NO: 160 | C_{S}A_{S}C_{S}a_{S}c_{S}a_{S}c_{S}t_{S}t_{S}t_{S}g_{S}c_{S}c_{S}C_{S}T_{S}C |
| SEQ ID NO: 161 | C_{S}A_{S}G_{S}t_{S}t_{S}c_{S}c_{S}a_{S}a_{S}a_{S}g_{S}a_{S}c_{S}A_{S}C_{S}C |
| SEQ ID NO: 162 | T_{S}G_{S}G_{S}c_{S}a_{S}a_{S}t_{S}t_{S}t_{S}g_{S}t_{S}a_{S}c_{S}T_{S}C_{S}C |
| SEQ ID NO: 163 | G_{S}T_{S}G_{S}t_{S}g_{S}t_{S}g_{S}t_{S}a_{S}t_{S}t_{S}t_{S}c_{S}C_{S}C_{S}A |
| SEQ ID NO: 164 | C_{S}C_{S}C_{S}t_{S}c_{S}t_{S}g_{S}a_{S}t_{S}g_{S}a_{S}c_{S}t_{S}C_{S}T_{S}G |
| SEQ ID NO: 165 | C_{S}A_{S}T_{S}a_{S}c_{S}t_{S}c_{S}c_{S}t_{S}c_{S}a_{S}t_{S}c_{S}T_{S}T_{S}C |
| SEQ ID NO: 166 | C_{S}C_{S}A_{S}c_{S}c_{S}a_{S}c_{S}a_{S}a_{S}a_{S}g_{S}t_{S}t_{S}A_{S}T_{S}G |
| SEQ ID NO: 167 | C_{S}A_{S}T_{S}c_{S}a_{S}c_{S}t_{S}c_{S}t_{S}g_{S}g_{S}t_{S}g_{S}T_{S}G_{S}T |
| SEQ ID NO: 168 | G_{S}A_{S}C_{S}a_{S}t_{S}c_{S}a_{S}c_{S}t_{S}c_{S}t_{S}g_{S}g_{S}T_{S}G_{S}T |

### 6.4. Example 4

### In Vitro Model: Cell Culture

The effect of antisense oligonucleotides on target nucleic acid expression can be tested in any of a variety of cell types provided that the target nucleic acid is present at measurable levels. The target can be expressed endogenously or by transient or stable transfection of a nucleic acid encoding said target. The expression level of target nucleic acid can be routinely determined using, for example, Northern blot analysis, Real-Time PCR, or ribonuclease protection assays. The following cell types are provided for illustrative purposes, but other cell types can be routinely used, provided that the target is expressed in the chosen cell type.

Cells were cultured in the appropriate medium as described below and maintained at 37° C. at 95-98% humidity and 5% CO₂. Cells were routinely passaged 2-3 times weekly.

15PC3: The human prostate cancer cell line 15PC3 was cultured in DMEM (Sigma)+10% fetal bovine serum (FBS)+2 mM Glutamax I+gentamicin (25 μg/ml).

HUH7: The human hepatocarcinoma cell line was cultured in DMEM (Sigma)+10% fetal bovine serum (FBS)+2 mM Glutamax I+gentamicin (25 μg/ml)+1× Non Essential Amino Acids.

### 6.5. Example 5

### In Vitro Model: Treatment with Antisense Oligonucleotides

The cells were treated with oligonucleotides using the cationic liposome formulation LipofectAMINE 2000 (Gibco) as transfection vehicle. Cells were seeded in 6-well cell culture plates (NUNC) and treated when 80-90% confluent. Oligomer concentrations ranged from 1 nM to 25 nM final concentration. Formulation of oligomer-lipid complexes was carried out essentially as described by the manufacturer using serum-free OptiMEM (Gibco) and a final lipid concentration of 5 μg/mL LipofectAMINE 2000. Cells were incubated at 37° C. for 4 hours and treatment was stopped by removal of oligomer-containing culture medium. Cells were washed and serum-containing medium was added. After oligomer treatment, cells were allowed to recover for 20 hours before they were harvested for RNA analysis.

### 6.6. Example 6

### In Vitro Model: Extraction of RNA and cDNA Synthesis

Total RNA was extracted from cells transfected as described above and using the Qiagen RNeasy kit (Qiagen cat. no. 74104) according to the manufacturer's instructions. First strand synthesis was performed using Reverse Transcriptase reagents from Ambion according to the manufacturer's instructions.

For each sample 0.5 μg total RNA was adjusted to (10.8 μl) with RNase free H₂O and mixed with 2 μl random decamers (50 μM) and 4 μl dNTP mix (2.5 mM each dNTP) and heated to 70° C. for 3 min after which the samples were rapidly cooled on ice. After cooling the samples on ice, 2 μl 10× Buffer RT, 1 μl MMLV Reverse Transcriptase (100 U/μl) and 0.25 μl RNase inhibitor (10 U/μl) was added to each sample, followed by incubation at 42° C. for 60 min, heat inactivation of the enzyme at 95° C. for 10 min, and then cooling the sample to 4° C.

### 6.7. Example 7

### In Vitro Model: Analysis of Oligonucleotide Inhibition of HER3, EGFR and HER2 Expression by Real-Time PCR

Antisense modulation of HER3, EGFR and HER2 expression can be assayed in a variety of ways known in the art. For example, HER3, EGFR and HER2 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA. Methods of RNA isolation and RNA analysis, such as Northern blot analysis, are routine in the art and are taught in, for example, Current Protocols in Molecular Biology, John Wiley and Sons.

Real-time quantitative (PCR) can be conveniently accomplished using the commercially available Multi-Color Real Time PCR Detection System, available from Applied Biosystem.

Real-time Quantitative PCR Analysis of HER3, EGFR and HER2 mRNA Levels

The sample content of human HER3, EGFR and HER2 mRNA was quantified using the human HER3, EGFR and HER2 ABI Prism Pre-Developed TaqMan Assay Reagents (Applied Biosystems cat. no. Hs00951444_ml (HER3), Hs00193306_ml (EGFR) and Hs00170433_ml (HER2) according to the manufacturer's instructions.

Glyceraldehyde-3-phosphate dehydrogenase (GAPDH) mRNA quantity was used as an endogenous control for normalizing any variance in sample preparation. The sample content of human GAPDH mRNA was quantified using the human GAPDH ABI Prism Pre-Developed TaqMan Assay Reagent (Applied Biosystems cat. no. 4310884E) according to the manufacturer's instructions.

Real-time Quantitative PCR is a technique well known in the art and is taught in for example in Heid et al. Real time quantitative PCR, Genome Research (1996), 6: 986-994.

### Real Time PCR

The cDNA from the first strand synthesis performed as described in Example 5 was diluted 2-20 times, and analyzed by real time quantitative PCR using Taqman 7500 FAST or 7900 FAST from Applied Biosystems. The primers and probe were mixed with 2× Taqman Fast Universal PCR master mix (2×) (Applied Biosystems Cat. #4364103) and added to 4 μl cDNA to a final volume of 10 μl. Each sample was analysed in duplicate. Assaying 2-fold dilutions of a cDNA that had been prepared on material purified from a cell line expressing the RNA of interest generated standard curves for the assays. Sterile H₂O was used instead of cDNA for the no-template control. PCR program: 95° C. for 30 seconds, followed by 40 cycles of 95° C., 3 seconds, 60° C., 20-30 seconds. Relative quantities of target mRNA sequence were determined from the calculated Threshold cycle using the Applied Biosystems Fast System SDS Software Version 1.3.1.21. or SDS Software Version 2.3.

### 6.8. Example 8

### In Vitro Analysis: Antisense Inhibition of Human HER3, EGFR and HER2 Expression by Oligonucleotide Compounds

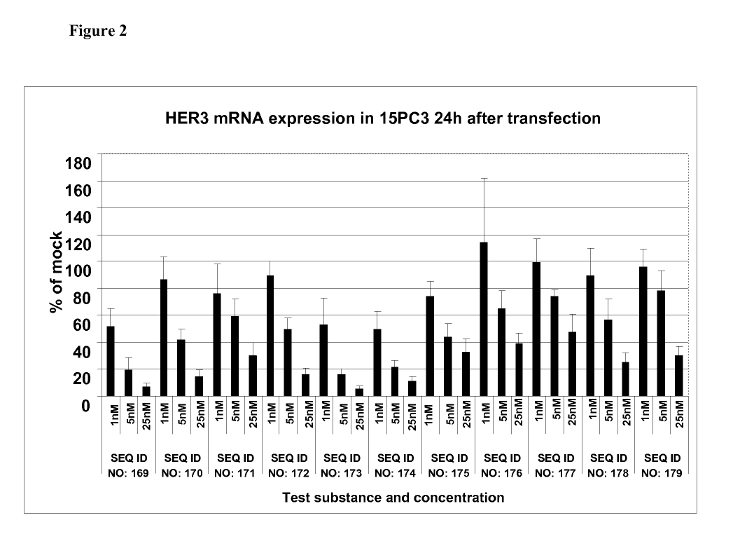

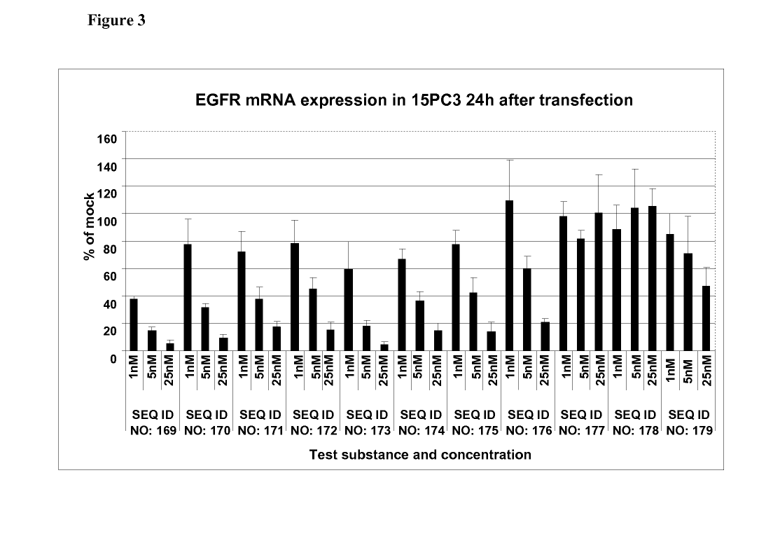

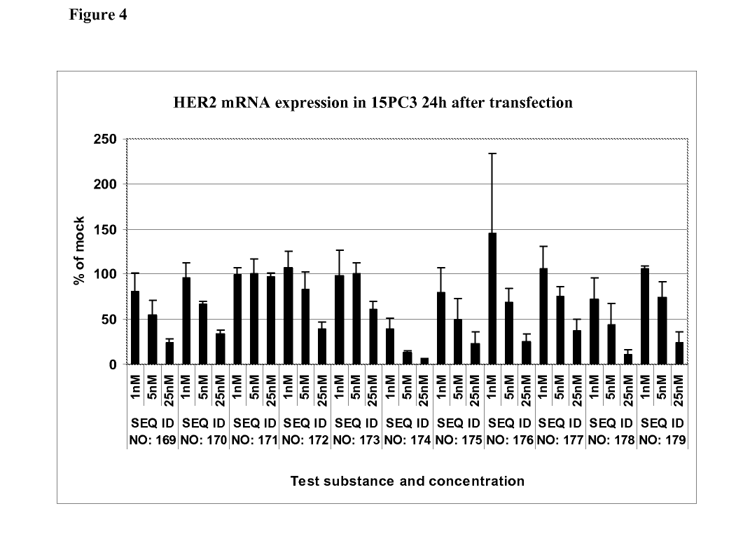

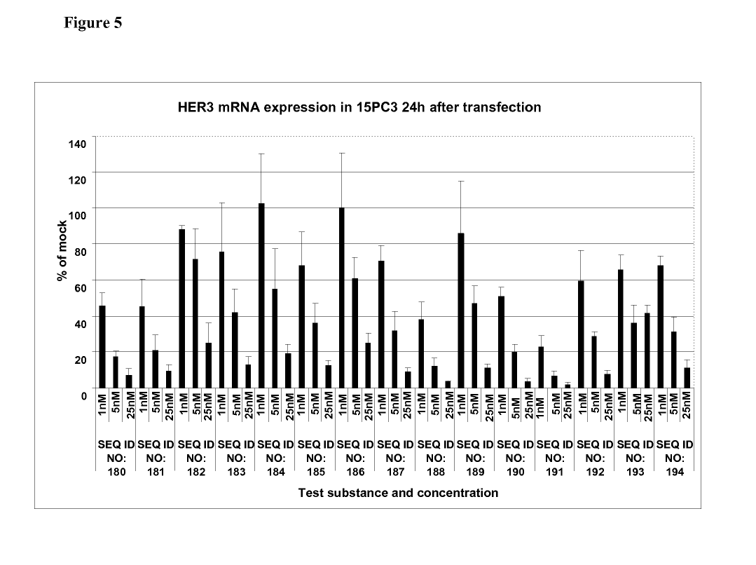

Oligonucleotides presented in Table 4 were evaluated for their potential to down-regulate HER3, EGFR and HER2 mRNA at concentrations of 1, 5 and 25 nM in 15PC3 cells (or HUH-7 as indicated by *) (see FIGS. 2, 3, 4 and 5). SEQ ID NOs: 235 and 236 were used as scrambled controls.

The data in Table 4 are presented as percentage down-regulation of mRNA relative to mock transfected cells at 25 nM. Lower-case letters represent DNA monomers, bold, upper-case letters represent β-D-oxy-LNA monomers. All cytosines in LNA monomers are 5-methylcytosines. Subscript “s” represents a phosphorothioate linkage.

TABLE 4
Inhibition of human HER3, EGFR and HER2 expression by antisense
oligonucleotides

| Test substance | Sequence (5′-3′) | HER3 | EGFR | HER2 |
|---|---|---|---|---|
| SEQ ID NO: 169 | G_{S}C_{S}T_{S}c_{S}c_{S}a_{S}g_{S}a_{S}c_{S}a_{S}t_{S}c_{S}a_{S}C_{S}T_{S}C | 93.4% | 95.2% | 75.8% |
| SEQ ID NO: 170 | C_{S}T_{S}C_{S}c_{S}a_{S}g_{S}a_{S}c_{S}a_{S}t_{S}c_{S}a_{S}c_{S}T_{S}C_{S}T | 85.8% | 91.5% | 65.8% |
| SEQ ID NO: 171 | C_{S}A_{S}G_{S}a_{S}c_{S}a_{S}t_{S}c_{S}a_{S}c_{S}t_{S}c_{S}t_{S}G_{S}G_{S}T | 70.6% | 84.2% | 2.8% |
| SEQ ID NO: 172 | A_{S}G_{S}A_{S}c_{S}a_{S}t_{S}c_{S}a_{S}c_{S}t_{S}c_{S}t_{S}g_{S}G_{S}T_{S}G | 84.2% | 86.2% | 61% |
| SEQ ID NO: 173 | A_{S}T_{S}A_{S}g_{S}c_{S}t_{S}c_{S}c_{S}a_{S}g_{S}a_{S}c_{S}a_{S}T_{S}C_{S}A | 94.5% | 96.4% | 39.2% |
| SEQ ID NO: 174 | T_{S}C_{S}A_{S}c_{S}a_{S}c_{S}c_{S}a_{S}t_{S}a_{S}g_{S}c_{S}t_{S}C_{S}C_{S}A | 88.8% | 86.4% | 94.8% |
| SEQ ID NO: 175 | C_{S}A_{S}T_{S}c_{S}c_{S}a_{S}a_{S}c_{S}a_{S}c_{S}t_{S}t_{S}g_{S}A_{S}C_{S}C | 65.5% | 86.1% | 76.9% |
| SEQ ID NO: 176 | A_{S}T_{S}C_{S}c_{S}a_{S}a_{S}c_{S}a_{S}c_{S}t_{S}t_{S}g_{S}a_{S}C_{S}C_{S}A | 61.6% | 79.4% | 74.8% |
| SEQ ID NO: 177 | C_{S}A_{S}A_{S}t_{S}c_{S}a_{S}t_{S}c_{S}c_{S}a_{S}a_{S}c_{S}a_{S}C_{S}T_{S}T | 51.1% | 0% | 63.4% |
| SEQ ID NO: 178 | T_{S}C_{S}A_{S}a_{S}t_{S}c_{S}a_{S}t_{S}c_{S}c_{S}a_{S}a_{S}c_{S}A_{S}C_{S}T | 76.7% | 0% | 88.6% |
| SEQ ID NO: 179 | C_{S}A_{S}T_{S}g_{S}t_{S}a_{S}g_{S}a_{S}c_{S}a_{S}t_{S}c_{S}a_{S}A_{S}T_{S}T | 70.5% | 52.6% | 75.6% |
| SEQ ID NO: 180 | T_{S}A_{S}G_{S}c_{S}c_{S}t_{S}g_{S}t_{S}c_{S}a_{S}c_{S}t_{S}t_{S}C_{S}T_{S}C | 92.8% | N.D. | N.D. |
| SEQ ID NO: 181 | A_{S}G_{S}A_{S}t_{S}g_{S}g_{S}c_{S}a_{S}a_{S}a_{S}c_{S}t_{S}t_{S}C_{S}C_{S}C | 90.6% | N.D. | N.D. |
| SEQ ID NO: 182 | C_{S}A_{S}A_{S}g_{S}g_{S}c_{S}t_{S}c_{S}a_{S}c_{S}a_{S}c_{S}a_{S}T_{S}C_{S}T | 74.6% | N.D. | N.D. |
| SEQ ID NO: 183 | A_{S}A_{S}G_{S}t_{S}c_{S}c_{S}a_{S}g_{S}g_{S}t_{S}t_{S}g_{S}c_{S}C_{S}C_{S}A | 85.9% | N.D. | N.D. |
| SEQ ID NO: 184 | C_{S}A_{S}T_{S}t_{S}c_{S}a_{S}a_{S}g_{S}t_{S}t_{S}c_{S}t_{S}t_{S}C_{S}A_{S}T | 81.1% | N.D. | N.D. |
| SEQ ID NO: 185 | C_{S}A_{S}C_{S}t_{S}a_{S}a_{S}t_{S}t_{S}t_{S}c_{S}c_{S}t_{S}t_{S}C_{S}A_{S}G | 89.1% | N.D. | N.D. |
| SEQ ID NO: 186 | G_{S}C_{S}C_{S}c_{S}a_{S}g_{S}c_{S}a_{S}c_{S}t_{S}a_{S}a_{S}t_{S}T_{S}T_{S}C | 79.9% | N.D. | N.D. |
| SEQ ID NO: 187 | C_{S}T_{S}T_{S}t_{S}g_{S}c_{S}c_{S}c_{S}t_{S}c_{S}t_{S}g_{S}c_{S}C_{S}A_{S}C | 90.4% | N.D. | N.D. |
| SEQ ID NO: 188 | C_{S}A_{S}C_{S}a_{S}c_{S}a_{S}c_{S}t_{S}t_{S}t_{S}g_{S}c_{S}c_{S}C_{S}T_{S}C | 96.1% | N.D. | N.D. |
| SEQ ID NO: 189 | C_{S}A_{S}G_{S}t_{S}t_{S}c_{S}c_{S}a_{S}a_{S}a_{S}g_{S}a_{S}c_{S}A_{S}C_{S}C | 88.9% | N.D. | N.D. |
| SEQ ID NO: 190 | T_{S}G_{S}G_{S}c_{S}a_{S}a_{S}t_{S}t_{S}t_{S}g_{S}t_{S}a_{S}c_{S}T_{S}C_{S}C | 95.7% | N.D. | N.D. |
| SEQ ID NO: 191 | G_{S}T_{S}G_{S}t_{S}g_{S}t_{S}g_{S}t_{S}a_{S}t_{S}t_{S}t_{S}c_{S}C_{S}C_{S}A | 97.7% | N.D. | N.D. |
| SEQ ID NO: 192 | C_{S}C_{S}C_{S}t_{S}c_{S}t_{S}g_{S}a_{S}t_{S}g_{S}a_{S}c_{S}t_{S}C_{S}T_{S}G | 92.3% | N.D. | N.D. |
| SEQ ID NO: 193 | C_{S}A_{S}T_{S}a_{S}c_{S}t_{S}c_{S}c_{S}t_{S}c_{S}a_{S}t_{S}c_{S}T_{S}T_{S}C | 64% | N.D. | N.D. |
| SEQ ID NO: 194 | C_{S}C_{S}A_{S}c_{S}c_{S}a_{S}c_{S}a_{S}a_{S}a_{S}g_{S}t_{S}t_{S}A_{S}T_{S}G | 87.5% | N.D. | N.D. |
| SEQ ID NO: 195 | C_{S}A_{S}T_{S}c_{S}a_{S}c_{S}t_{S}c_{S}t_{S}g_{S}g_{S}t_{S}g_{S}T_{S}G_{S}T | 64.4%* | N.D. | N.D. |
| SEQ ID NO: 196 | G_{S}A_{S}C_{S}a_{S}t_{S}c_{S}a_{S}c_{S}t_{S}c_{S}t_{S}g_{S}g_{S}T_{S}G_{S}T | 77.0%* | N.D. | N.D. |
| SEQ ID NO: 234 | T_{S}A_{S}g_{S}c_{S}c_{S}t_{S}g_{S}t_{S}c_{S}a_{S}C_{S}T_{S}T | | | |
| SEQ ID NO: 235 | C_{S}G_{S}T_{S}c_{S}a_{S}g_{S}t_{S}a_{S}t_{S}g_{S}c_{S}g_{S}A_{S}A_{S}T_{S}c | | | |
| SEQ ID NO: 236 | C_{S}G_{S}C_{S}A_{S}g_{S}a_{S}t_{S}t_{S}a_{S}g_{S}a_{S}a_{S}A_{S}C_{S}C_{S}t | | | |
| SEQ ID NO: 249 | T_{S}A_{S}G_{S}c_{S}c_{S}t_{S}t_{S}t_{S}g_{S}a_{S}c_{S}c_{S}t_{S}C_{S}T_{S}C | | | |

As shown in Table 4, oligonucleotides having the sequences shown in SEQ ID NOs: 169, 170, 173, 174, 180, 181, 183, 185, 187, 188, 189, 190, 191, 192 and 194 demonstrated about 85% or greater inhibition of HER3 mRNA expression at 25 nM in 15PC3 cells in these experiments, and are therefore preferred.

Also preferred are oligonucleotides based on the illustrated antisense oligomer sequences, for example varying the length (shorter or longer) and/or monomer content (e.g., the type and/or proportion of nucleoside analogue monomers), which also provide good inhibition of HER3 expression.

### 6.9. Example 9

### Apoptosis Induction by LNA Oligonucleotides

HUH7 cells were seeded in 6-well culture plates (NUNC) the day before transfection at a density of 2.5×10⁵ cells/well. The cells were treated with oligonucleotides using the cationic liposome formulation LipofectAMINE 2000 (Gibco) as transfection vehicle when 75-90% confluent. The oligomer concentrations used were 5 nM and 25 nM (final concentration in well). Formulation of oligomer-lipid complexes was carried out essentially as described by the manufacturer using serum-free OptiMEM (Gibco) and a final lipid concentration of 5 μg/mL LipofectAMINE 2000. Cells were incubated at 37° C. for 4 hours and treatment was stopped by removal of oligomer-containing culture medium. After washing with Optimem, 300 μl of trypsin was added to each well until the cells detached from the wells. The trypsin was inactivated by adding 3 ml HUH7 culture medium to the well and a single cell suspension was made by gently pipetting the cell suspension up and down. The scrambled oligomer SEQ ID NO: 235 was used as control.

Following this, 100 μl of the cell suspension was added to each well of a white 96-well plate from Nunc (cat #136101) (four plates were prepared, for measurement at different time points). The plates were then incubated at 37° C., 95% humidity and 5% CO₂ until the assays were performed.

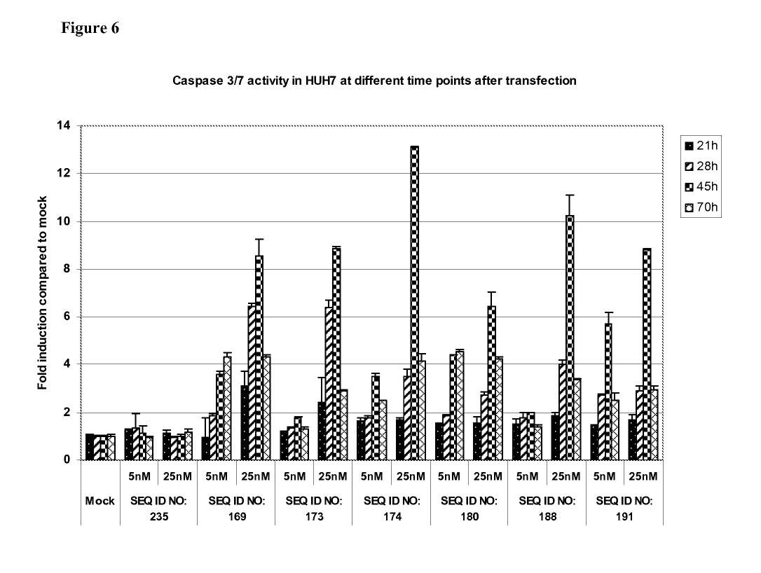

Caspase assay: The activities of apoptosis-specific caspases 3 and 7 were measured using a luminogenic Caspase-Glo 3/7-substrate assay (Cat#G8091 from Promega). The plate to be analyzed was equilibrated to room temperature for 15 min. The Caspase-Glo® 3/7 buffer was mixed with the Caspase-Glo® 3/7 substrate to form a Caspase-Glo® working solution which was equilibrated to room temperature. Then, 100 μl of the Caspase-Glo® working solution was carefully added to the medium in each well of the 96-well plate (avoiding bubbles and contamination between wells). The plate was carefully shaken for 1 min, after which it was incubated at room temperature for 1 h, protected from light. The caspase activity was measured as Relative Light Units per second (RLU/s) in a Luminoscan Ascent instrument (Thermo Labsystems). Data were correlated and plotted relative to an average value of the mock samples, which was set to 1. See FIG. 6.

### 6.10. Example 10

### In Vitro Inhibition of Proliferation Using LNA Oligonucleotides

HUH7 cells were transfected and harvested into a single cell suspension as described in Example 9. SEQ ID NO: 235 served as a scrambled control. Following harvesting, 100 μl of the cell suspension was added to each well of a 96-well plate (“Orange Scientific”) for MTS assay (four plates were prepared, for measurement at different time points). The plates were then incubated at 37° C., 95% humidity and 5% CO₂ until the assays were performed.

### Measurement of Proliferating Viable Cells (MTS Assay)

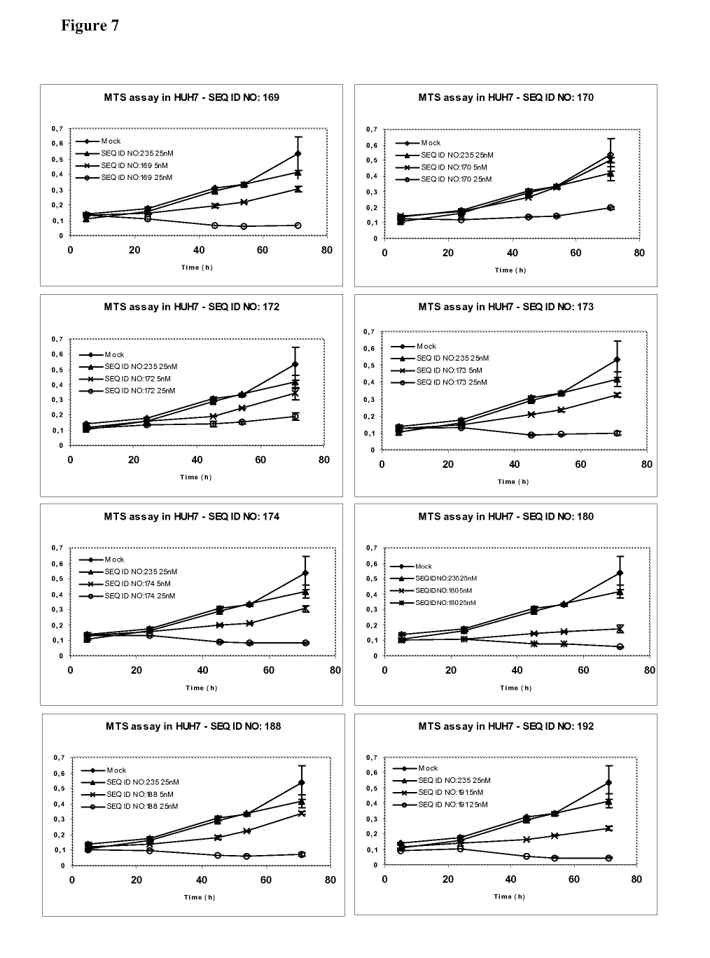

For the proliferation assay, 10 μl CellTiter 96® AQueous One Solution Cell Proliferation Assay (Promega, G3582) were added to the medium of each well of the 96-well plate, the plate was carefully shaken, and incubated at 37° C., 95% humidity and 5% CO₂ for 1 h before measurement. The absorbance was measured at 490 nm in a spectrophotometer and background for the assay was subtracted from wells containing only medium. The absorbance at 490 nm is proportional to the number of viable cells and was plotted over time for the mock transfected cells and for cells transfected with oligomers. See FIG. 7.

### 6.11. Example 11

### Evaluation of Target mRNA Knockdown In Vivo

To evaluate the knockdown efficacy of the HER3 oligomeric compounds in vivo, the female nude mice bearing 15PC3 xenografts developed by subcutaneous injection of 5×10⁶ cells/mouse into the right axillary flank, were injected intravenously with the oligomers at various doses and injection schedules (i.e. single dose, qd, q3d, q4d). Scrambled oligomer SEQ ID NO: 236 served as a negative control. 24 hours after the last injection, the mice were euthanized and liver and tumor tissues were collected in RNAlater solution (Ambion). Total RNA was purified from the tissues and the levels of HER3 mRNA were determined by quantitative reverse transcription-real time PCR (qRT-PCR) using the QuantiTect Probe RT-PCR kit (Cat#: 204443; Qiagen). GAPDH mRNA served as an internal control.

Mouse HER3:
probe: cca cac ctg gtc ata gcg gtg a,
primer-1: ctg ttt agg cca agc aga gg,
primer-2: att ctg aat cct gcg tcc ac.
Human HER3:
probe: cat tgc cca acc tcc gcg tg,
primer-1: tgc agt gga ttc gag aag tg,
primer-2: ggc aaa ctt ccc atc gta ga.
Human GAPDH:
probe: act ggc gct gcc aag gct gt,
primer-1: cca ccc aga aga ctg tgg at,
primer-2: ttc agc tca ggg atg acc tt.
Mouse GAPDH:
probe: agc tgt ggc gtg atg gcc gt,
primer-1: aac ttt ggc att gtg gaa gg,
primer-2: gga tgc agg gat gat gtt ct

200 ng of total RNA was used in the PCR reaction. The data analyses were performed by using the ABI-7500 PCR Fast System included software. See Table 5.

Data in Table 5 are presented as % HER3 mRNA levels relative to saline treated controls in liver and tumor samples after i.v. dosing of animals on 5 consecutive days with oligonucleotides in the doses indicated.

TABLE 5
Inhibition of HER3 mRNA in mouse liver and tumor

| | Dosage | HER3 mRNA | |
|---|---|---|---|
| | (mg/kg, i.v., | Liver (% of | |
| LNA ID | qd × 5) | Sal ctrl) | Tumor (%) |
| SEQ ID | 76.3 | 78 ± 17 | 100 ± 10.5 |
| NO: 236 | 60 | 86.5 ± 9.9 | 95.5 ± 12.7 |
| | 30 | 87.6 ± 19 | 101.2 ± 21.1 |
| | 22.9 | 81.4 ± 6.5 | 119.3 ± 24.9 |
| SEQ ID | 85.3 | 1 ± 0.3 | 25.8 ± 4.1 |
| NO: 180 | 66 | 6 ± 5.3 | 32.3 ± 9.7 |
| | 31.3 | 1.6 ± 0.3 | 37 ± 5.8 |
| | 25.6 | 3 ± 0.3 | 65 ± 20.2 |
| | 19.8 | 1.7 ± 0.6 | 83.1 ± 19.5 |
| SEQ ID | 37.7 | 20.7 ± 9.8 | 77 ± 10 |
| NO: 169 | 11.3 | 10.2 ± 5.5 | ND |
| SEQ ID | 32.4 | 7.4 ± 5.2 | 78.1 ± 15.3 |
| NO: 172 | 9.7 | 12.2 ± 5.9 | ND |

### 6.12. Example 12

### Evaluation of Tumor Growth Inhibition

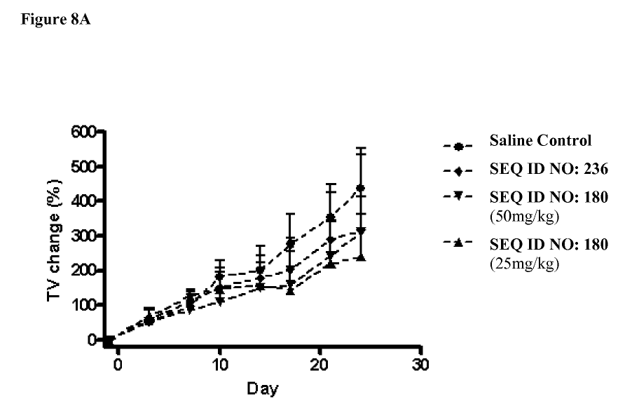

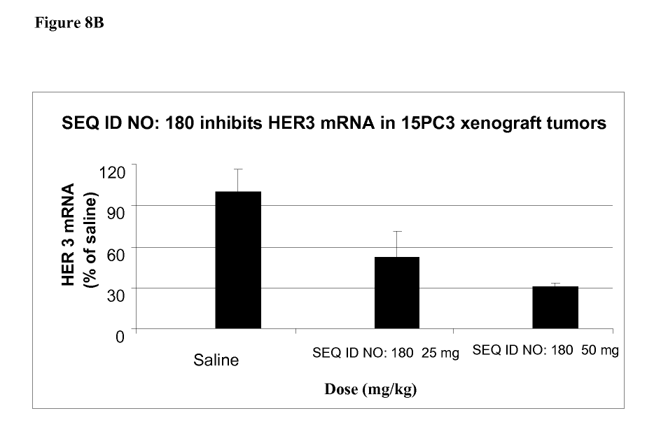

The ability of the HER3 specific LNAs to inhibit tumor growth in vivo was evaluated in nude female mice bearing 15PC3 xenografts. 15PC3 human prostate tumor model was developed by subcutaneously injection of 5×10⁶ cells/mouse into the right axillary flank. The tumor volume was determined by measuring two dimensions with callipers and calculated using the formula: tumor volume=(length×width)/2). When the tumors reached an average volume of 70-100 mm³, the mice bearing tumors were divided into treatment and control groups. The mice were injected intravenously with 25 and 50 mg/kg of SEQ ID NO: 180 respectively, with a q3d×10 schedule. Saline or scrambled oligonucleotide having SEQ ID NO: 236 served as a control. The body weights and tumor sizes of the mice were measured twice weekly. The toxicity was estimated by clinical observation, clinical chemistry and histopathological examination. Tumor HER3 mRNA was measured by QPCR as described in Example 11. See FIGS. 8A and 8B.

### 6.13. Example 13

### Inhibition of HER3 mRNA in Mouse Liver

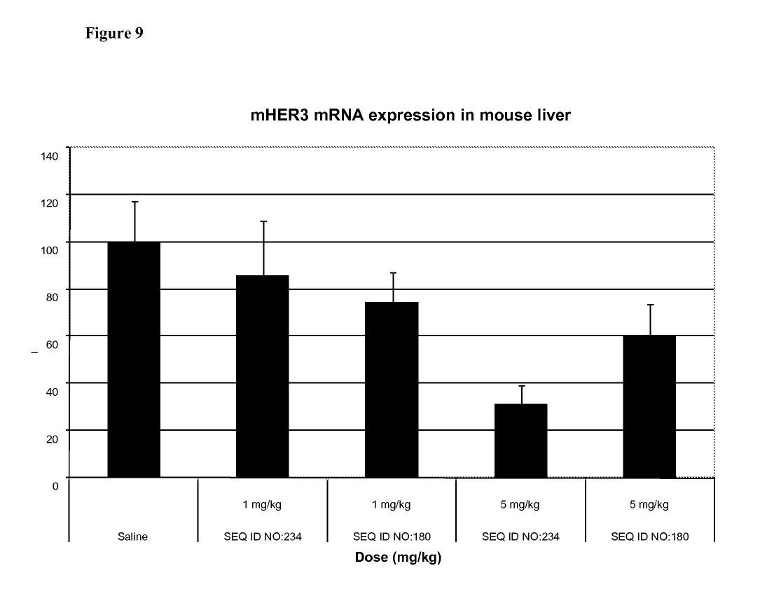

NMRI mice were dosed i.v. with 1 or 5 mg/kg oligonucleotides on three consecutive days (group size of 5 mice). The antisense oligonucleotides (SEQ ID NO: 180 and SEQ ID NO: 234) were dissolved in 0.9% saline (NaCl). Animals were sacrificed 24 h after last dosing and liver tissue was sampled and stored in RNA later (Ambion) until RNA extraction and QPCR analysis. Total RNA was extracted and HER3 mRNA expression in liver samples was measured by QPCR as described in Example 7 using a mouse HER3 QPCR assay (cat. no. Mm01159999_ml, Applied Biosystems). Results were normalized to mouse GAPDH (cat. no. 4352339E, Applied Biosystems) and plotted relative to saline treated controls (see FIG. 9).

### 6.14. Example 14

### Preparation of Conjugates of Oligomers with Polyethylene Glycol

The oligomers having sequences shown as SEQ ID NO: 141 or SEQ ID NO: 152 are functionalized on the 5′ terminus by attaching an aminoalkyl group, such as hexan-1-amine blocked with a blocking group such as Fmoc to the 5′ phosphate groups of the oligomers using routine phosphoramidite chemistry, oxidizing the resultant compounds, deprotecting them and purifying them to achieve the functionalized oligomers, respectively, having the formulas (IA) and (IB):

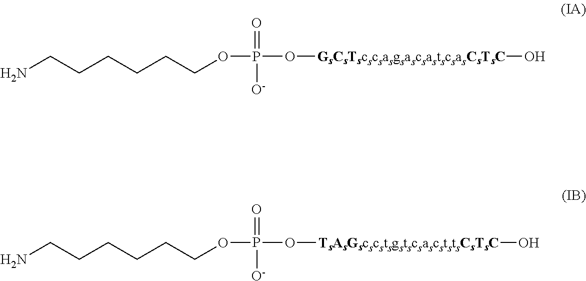

wherein the bold uppercase letters represent nucleoside analogue monomers, lowercase letters represent DNA monomers, and the subscript “s” represents a phosphorothioate linkage.

A solution of activated PEG, such as the one shown in formula (II):

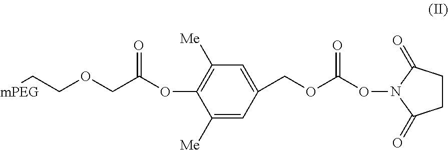

wherein the PEG moiety has an average molecular weight of 12,000, and each of the compounds of formulas (IA) and (IB) in PBS buffer are stirred in separate vessels at room temperature for 12 hours. The reaction solutions are extracted three times with methylene chloride and the combined organic layers are dried over magnesium sulphate and filtered and the solvent is evaporated under reduced pressure. The resulting residues are dissolved in double distilled water and loaded onto an anion exchange column.

Unreacted PEG linker is eluted with water and the products are eluted with NH₄HCO₃ solution. Fractions containing pure products are pooled and lypophilized to yield the conjugates SEQ ID NOs: 141 and 152, respectively as show in formulas (IIIA) and (IIIB):

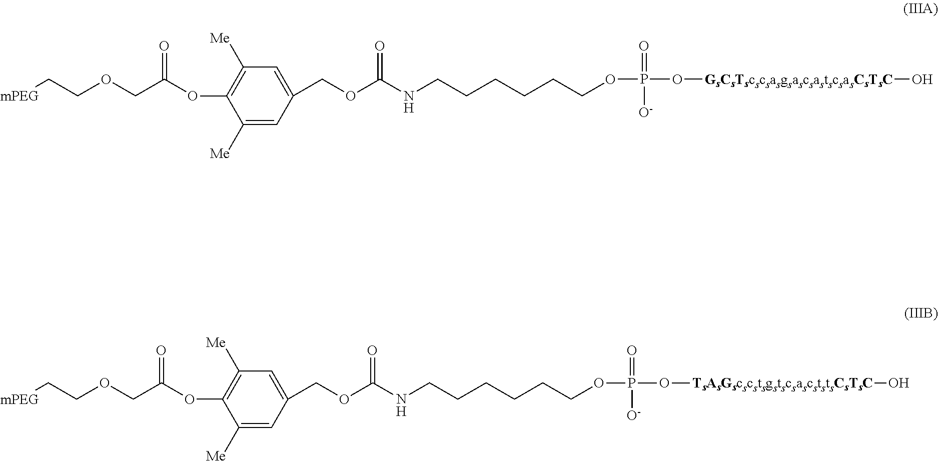

wherein each of the oligomers of SEQ ID NOs: 141 and 152 is attached to a PEG polymer having average molecular weight of 12,000 via a releasable linker.

Chemical structures of PEG polymer conjugates that can be made with oligomers having sequences shown in SEQ ID NOs: 169, 180 and 234 using the process described above are respectively shown in formulas (WA), (IVB) and (IVC):

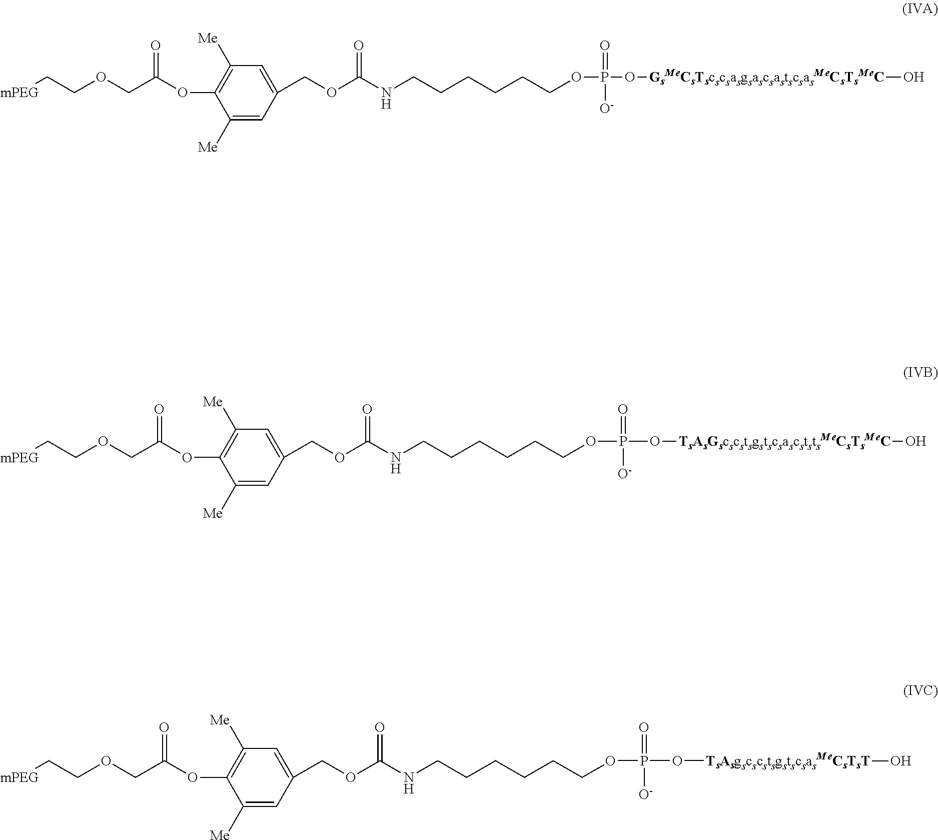

wherein bold uppercase letters represent beta-D-oxy-LNA monomers, lowercase letters represent DNA monomers, the subscript “s” represents a phosphorothioate linkage and ^{Me}C represent 5-methylcytosine.

Activated oligomers that can be used in this process to respectively make the conjugates shown in formulas (IVA), (IVB) and (IVC) have the chemical structures shown in formulas (VA), (VB) and (VC):

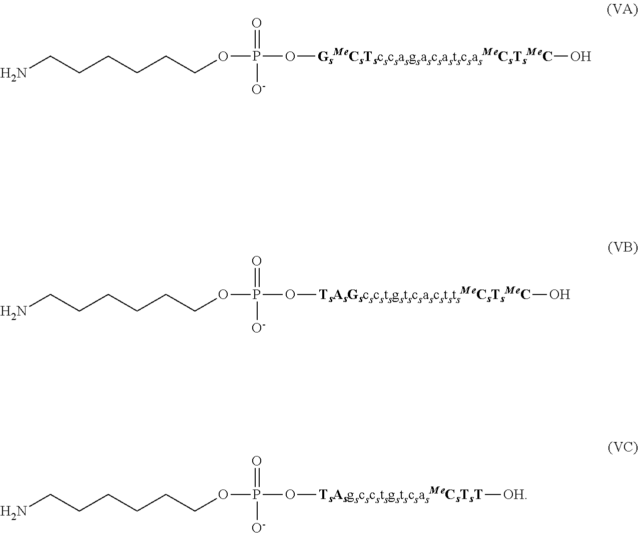

### 6.15. Example 15

### Evaluation of Target mRNA Knockdown In Vivo with Different Dosing Cycle

The knockdown efficacy of oligomers was evaluated in vivo in nude mice bearing xenograft tumors derived from 15PC3 cells or A549 cells (NSCLC) or N87 cells (gastric carcinoma) using a similar protocol to the one described above in Example 11. Oligomers were administered by injection every third day in 2-4 doses. Tissues were harvested 3 or 4 days after the last injection.

Data in Tables 6 and 7 are presented as % HER3 mRNA or HIF-1 alpha mRNA relative to saline treated controls in liver and tumor samples after i.v. dosing of animals with the indicated oligomers.

TABLE 6
Inhibition of ErbB3 mRNA in mouse liver and xenograft
tumor derived from 15PC3 cell (3-5 mice/group)

| | Dosage | Tumor | | Liver |
|---|---|---|---|---|
| Treatment | (mg/kg) | HER3 (%) | Hif1A (%) | HER3 (%) |
| Saline | 0 × 4 | 100 ± 10 | 100 ± 8 | 100 ± 18 |
| SEQ ID No: | 76.3 × 4 | 106 ± 6.6 | 101 ± 13.8 | 115.9 ± 26.3 |
| 236 | | | | |
| SEQ ID No: | 37.7 × 4 | 81.6 ± 12.7 | 94.6 ± 19.6 | 39 ± 4.6 |
| 169 | | | | |
| SEQ ID No: | 32.4 × 4 | 107.3 ± 17 | 100.3 ± 7.5 | 44.3 ± 10.6 |
| 172 | | | | |
| SEQ ID No: | 60.2 × 2 or 3 | 47.1 ± 2.2 | 101 ± 7.3 | 6.9 ± 3.6 |
| 180 | 60.2 × 4 | 54.2 ± 9.1 | ND | 31.8 ± 5 |

The observed knockdown effects of the oligomers having the sequences of SEQ ID NO: 169 and SEQ ID NO: 180 are not unique to 15PC3 tumor cells, since similar effects were observed in the tumors derived from A549 (NSCLC) and N87 (gastric carcinoma) cells. See Table 7, below.

TABLE 7
Inhibition of ErbB3 mRNA in mouse liver and xenograft
tumor derived from N87 cell (3 mice/group)

| Xenograft | | Dosage | HER3 (% saline control) | |
|---|---|---|---|---|
| model | Treatment | mg/kg | Tumor | Liver |
| A549 | Saline | 0 × 3 | 100 ± 20.9 | 100 ± 4.8 |
| | SEQ ID No: | 35, q4d × 3 | 87.6 ± 11.9 | 97.5 ± 21.2 |
| | 236 | | | |
| | SEQ ID No: | 35, q4d × 3 | 54.6 ± 15.2 | 31.8 ± 5.7 |
| | 180 | | | |
| N87 | Saline | 0 × 5 | 100 ± 8.2 | 100 ± 9.4 |
| | SEQ ID No: | 25, q3d × 5 | 99.0 ± 8.9 | 123 ± 4.5 |
| | 249 | | | |
| | SEQ ID No: | 25, q3d × 5 | 46.6 ± 13.4 | 24.7 ± 3.1 |
| | 180 | | | |

### 6.16. Example 16

### Generation of a Gefitinib-Resistant Cell Line

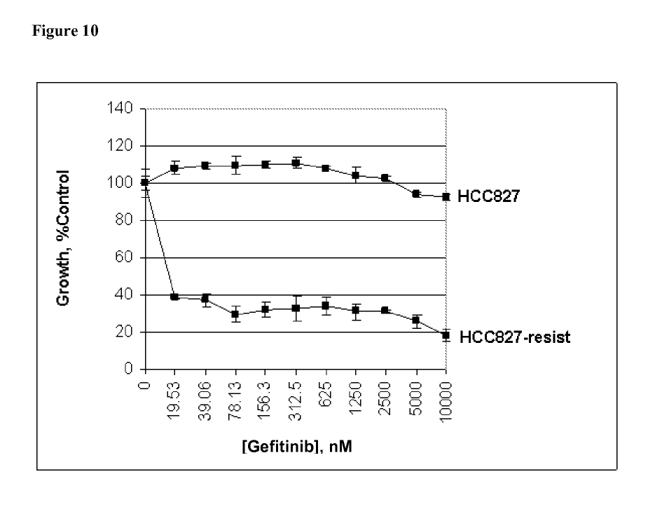

HCC827 lung adenocarcinoma cells (ATCC CRL-2868) were maintained at 37° C. in a humidified atmosphere of 5% CO₂ and 95% air in RPMI medium supplemented with 10% fetal bovine serum. To generate gefitinib resistance, cells were treated with increasing amounts of gefitinib (up to 500 nM) for a period of 3 months. At the end of the 3 month period, cell proliferation was tested comparing both the parental and HCC827R gefitinib resistant cells using an MTT ((3-(4,5-Dimethylthiazol-2-yl)-2,5-diphenyltetrazolium bromide) assay. The results show that HCC827R cells are resistant to gefitinib even at the highest concentration tested (10 μM). (FIG. 10)

### 6.17. Example 17

### Characterization of Gefitinib-Resistant Cell Line

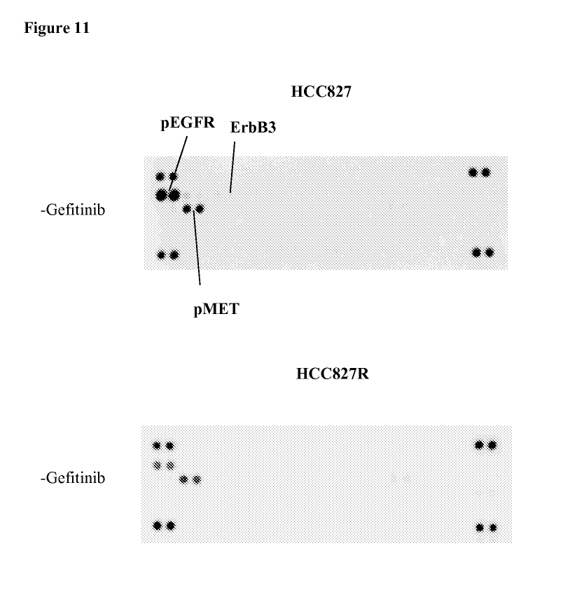

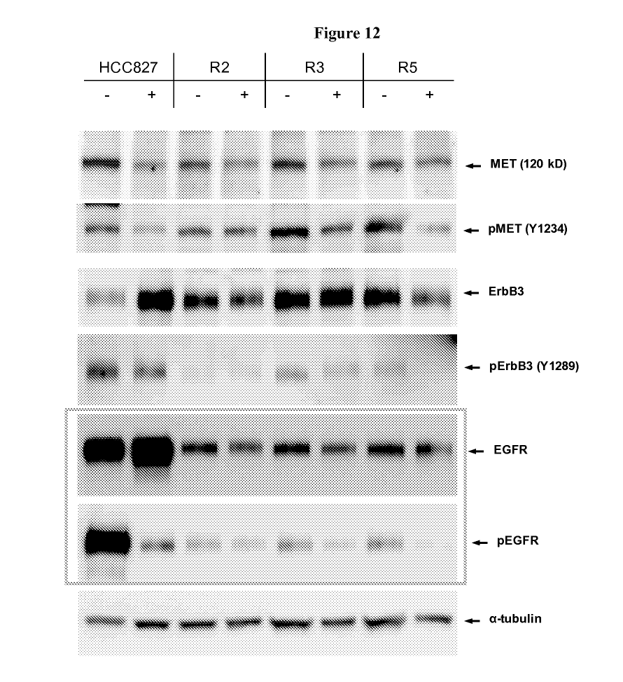

Expression levels and phosphorylation status of receptor tyrosine kinases (“RTK”) in HCC827 and the gefitinib-resistant cells, HCC827R, were profiled using the RTK Antibody Array kit (R&D Systems, Inc., Minneapolis, Minn.). Briefly, cells were solubilized in the lysis buffer and total protein concentration in the cell lysates was determined 500 ug of total protein was diluted in the array incubation buffer, incubated with the array membrane, and processed according to the protocol provided by the manufacturer. The final imaging result (FIG. 11) shows that phosphorylated EGFR levels in the HCC827R cells were much lower than those of the parent.

### Western Blot Analysis

HCC827 and gefitinib-resistant clones (R2, R3, and R5) were cultured in medium with (“+”) or without (“−”) 1 μM of gefitinib for 24 h. Cell lysates were then prepared and total protein concentration was determined Approximately 15 μg/lane of protein were electrophoresed in 8% SDS-PAGE gels and transferred to PVDF using a BioRad liquid transfer apparatus. The western analysis was performed with the appropriate horseradish peroxidase-conjugated secondary antibodies (Transduction Labs) and enhanced chemiluminescence reagents (SuperSignal, Pierce). The primary antibodies (Abs) used include: anti-Met monoclonal Ab (25H2), anti-phosphor-Met(Y1234) rabbit monoclonal Ab (D26), and anti-phosphor-ErbB3(Y1289) rabbit monoclonal Ab (21D3), from Cell Signaling; anti-ErbB3 Ab (sc285) from Santa Crutz; anti-phosphor-Met(Y1349) Ab (Ab47606R), anti-phosphor-EGFR rabbit monoclonal Ab (Ab40815), and anti-EGFR Ab, from Abcam; and a horseradish peroxidase-conjugated anti-tubulin Ab for loading control.

Data show that levels of unphosphorylated and phosphorylated EGFR are significantly reduced in HCC827 gefitinib-resistant clones, either in the presence (“+”) or absence (“−”) of gefitinib, as compared to the levels of unphosphorylated and phosphorylated EGFR in untreated (“−”) parent cells. In contrast, the levels of ErbB3 or MET, which are also involved in the EGFR signaling pathway, are not significantly decreased in the resistant clones compared to the parent cells. These findings indicate that down-regulation of EGFR may be a mechanism by which some cancer cells acquire resistance to gefitinib.

### 6.18. Example 18

### Effect of Oligomer on Gefitinib-Resistant Cells

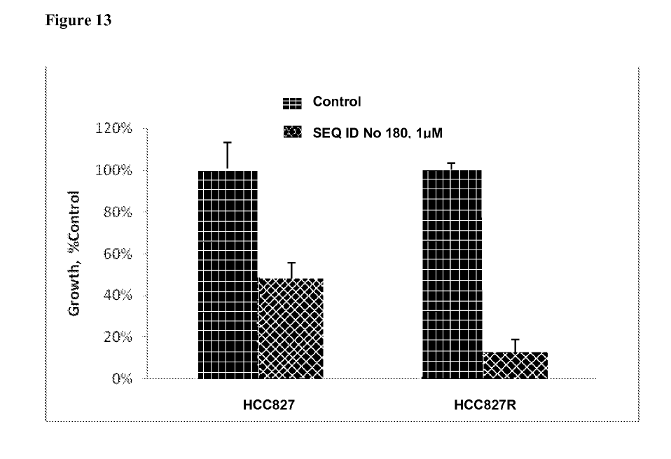

HCC287 and HCC287R cells were plated in duplicate at 200 cells/well of a 6-well plate and incubated for 24 hours. Cells were treated with 1 μM of ON180 (SEQ ID NO: 180) and incubated for 10 days, after which cells were stained with MTT and the number of colonies counted. Percent of control was calculated for both HCC827 and HCC727R cells. Results shown in FIG. 13 indicate that oligonucleotide ON180 is significantly more effective in down-regulating gefitinib-resistant cells (greater than 80% reduction in cell growth as compared to the untreated control) than in down-regulating growth of HCC287 gefitinib-sensitive cells (about 50% reduction in cell growth as compared to the untreated control).

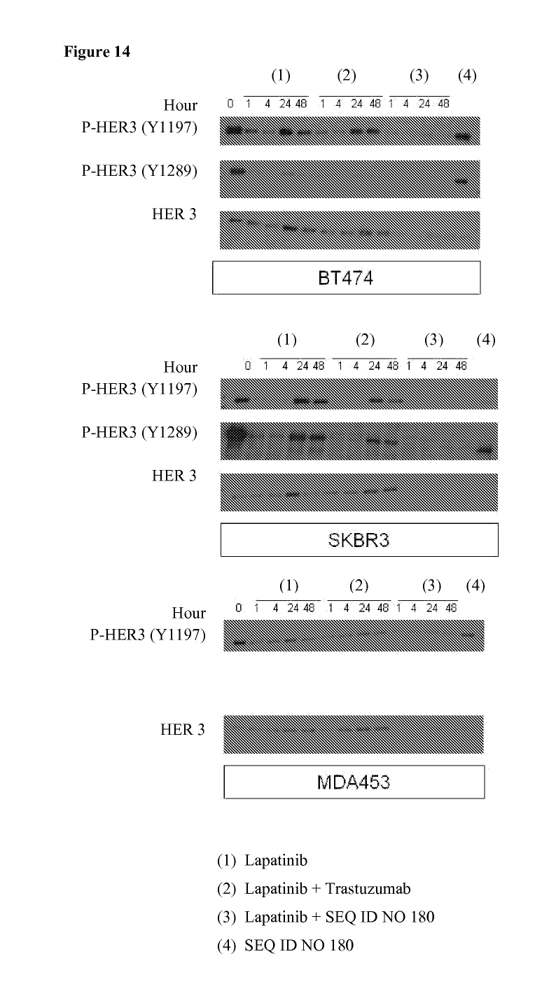

Still further aspects and embodiments of the invention are illustrated with respect to FIGS. 14-16.

FIG. 14 shows that HER3 expression-reducing LNA oligomer, but not trastuzumab, is able to prevent feedback upregulation of HER3 and P-HER3 expression by lapatinib in three human breast cancer cell lines, BT474, SKBR3 and MDA453. The expression level of HER3, P-HER3 (Y1197) and P-HER3 (Y1289) is shown at 0, 1, 4, 24 and 48 hours as indicated for lapitinib-only treated cells (1), lapatinib plus trastuzumab-treated cells (2), lapitinib plus SEQ ID NO: 180-treated cells (3) and SEQ ID NO: 180-only treated cells (4). Lapatinib was used at a concentration of 1 μM, trastuzumab at a concentration of 10 μg/ml, and SEQ ID NO: 180 at a concentration of 5 μM.

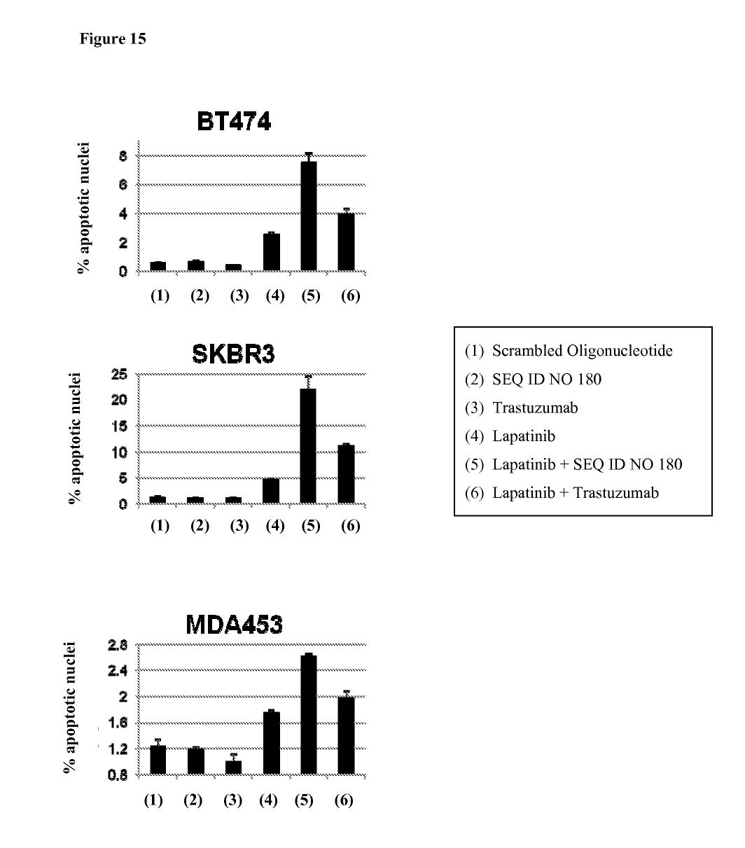

FIG. 15 shows that synergistic promotion of apoptosis in three human breast cancer cell lines is greater for a combination of lapatinib and a HER3 expression-reducing LNA oligomer than for a combination of lapatinib and trastuzumab. The figure shows the results of an ApoBrdU apoptosis assay performed for each of the three cells lines (same lines as in FIG. 14). Cells were treated at 48 hours with lapatinib and/or trastuzumab. At 72 hours, the cells were serum starved and treated with SEQ ID NO: 180 or a randomized control oligomer. For each of the cell lines, treatments were randomized oligonucleotide control-only (1), SEQ ID NO: 180-only (2), trastuzumab-only (3), lapatinib-only (4), lapatinib plus SEQ ID NO: 180 (5), and lapatinib plus trastuzumab (6). Lapatinib was used at a concentration of 1 μM, trastuzumab at a concentration of 10 μg/ml, and SEQ ID NO: 180 at a concentration of 5 μM.

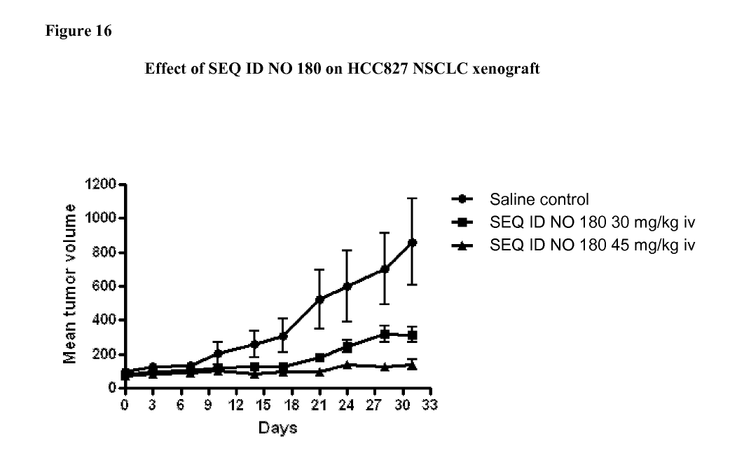

FIG. 16 shows that SEQ ID NO: 180 inhibits tumor growth in an in vivo mouse xenograft model of the human non-small cell lung cancer using the HCC827 human cell line. Mean tumor volume was reduced 65.5% vs. saline control for treatment with 30 mg/kg SEQ ID NO: 180 i.v. (intravenous) at approximately 31 days and was reduced 81.3% vs. saline control for treatment with 45 mg/kg SEQ ID NO: 180 i.v. at approximately 31 days. N=6.

### SPECIFIC EMBODIMENTS, CITATION OF REFERENCES

The present invention is not to be limited in scope by the specific embodiments described herein. Indeed, various modifications within the scope of the invention, in addition to those described herein, will become apparent to those skilled in the art from the foregoing description and accompanying figures. Moreover, features described in connection with one embodiment of the invention may be used in conjunction with other embodiments, even if not explicitly stated above.

Various references, including patent applications, patents, and scientific publications, are cited herein; the disclosure of each such reference is hereby incorporated herein by reference in its entirety.

1. A method for treating a cancer in a mammal, comprising:
administering a protein tyrosine kinase inhibitor to the mammal; and
administering to the mammal at least one antisense oligomer or conjugate thereof that reduces the expression of HER3,
wherein the inhibitory activity of the protein tyrosine kinase inhibitor and the reduction of expression of HER3 are temporally overlapping.

2. The method of claim 1, wherein the cancer is breast cancer.

3. The method of claim 1, wherein the cancer is at least partially resistant to treatment with the protein kinase inhibitor and said resistance is at least partially reversed by administering the at least one antisense oligomer or conjugate thereof that reduces the expression of HER3.

4. The method of claim 1, wherein the cancer is breast cancer.

5. The method of claim 1, wherein the protein tyrosine kinase inhibitor is selected from the group consisting of gefitinib, imatinib, erlotinib, lapatinib, canertinib and sorafenib.

6. The method of claim 1, wherein the at least one antisense oligomer or conjugate thereof comprises a therapeutically effective amount of
(SEQ ID NO: 180)
5′-T_{S}A_{S}G_{S}c_{S}c_{S}t_{S}g_{S}t_{S}c_{S}a_{S}c_{S}t_{S}t_{S}^{Me}C_{S}T_{S}^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base,
or a conjugate thereof.

7. (canceled)

8. (canceled)

9. A method for treating a cancer in a mammal, comprising:
administering a HER2 inhibitor to the mammal; and
administering to the mammal at least one antisense oligomer or conjugate thereof that reduces the expression of HER3,
wherein the inhibitory activity of the HER2 inhibitor and the reduction of expression of HER3 are temporally overlapping.

10. The method of claim 9, wherein the cancer is breast cancer.

11. The method of claim 9, wherein the cancer is at least partially resistant to treatment with the HER2 inhibitor and said resistance is at least partially reversed by administering the at least one antisense oligomer or conjugate thereof that reduces the expression of HER3.

12. The method of claim 9, wherein the cancer is breast cancer.

13. The method of claim 9, wherein the HER2 inhibitor is selected from the group consisting of trastuzumab and pertuzumab.

14. The method of claim 9, wherein the at least one antisense oligomer or conjugate thereof comprises a therapeutically effective amount of
(SEQ ID NO: 180)
5′-T_{S}A_{S}G_{S}c_{S}c_{S}t_{S}g_{S}t_{S}c_{S}a_{S}c_{S}t_{S}t_{S}^{Me}C_{S}T_{S}^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base,
or a conjugate thereof.

15. (canceled)

16. (canceled)

17. A method of treating cancer in a mammal, comprising administering to said mammal an effective amount of an oligomer consisting of 10 to 50 contiguous monomers wherein adjacent monomers are covalently linked by a phosphate group or a phosphorothioate group,
wherein said oligomer comprises a first region of at least 10 contiguous monomers;
wherein at least one monomer of said first region is a nucleoside analogue;
wherein the sequence of said first region is at least 80% identical to the reverse complement of the best-aligned target region of a mammalian HER3 gene or a mammalian HER3 mRNA; and
wherein the cancer is resistant to treatment with a protein tyrosine kinase inhibitor.

18. The method of claim 17, wherein the cancer is selected from non-Hodgkin's lymphoma, Hodgkin's lymphoma, acute lymphocytic leukemia, acute myelocytic leukemia, chronic myeloid leukemia, chronic lymphocytic leukemia, multiple myeloma, colon carcinoma, rectal carcinoma, pancreatic cancer, breast cancer, ovarian cancer, prostate cancer, renal cell carcinoma, hepatoma, bile duct carcinoma, choriocarcinoma, cervical cancer, testicular cancer, non-small cell lung cancer, bladder carcinoma, melanoma, head and neck cancer, brain cancer, cancers of unknown primary site, neoplasms, cancers of the peripheral nervous system, cancers of the central nervous system, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma.

19. The method according to claim 17, wherein the sequence of the first region of the oligomer is at least 80% identical to the sequence of a region of at least 10 contiguous monomers present in SEQ ID NOs: 1-140 and 169-234.

20. The method according to claim 19, wherein the sequence of the first region of the oligomer is at least 80% identical to the sequence of a region of at least 10 contiguous monomers present in SEQ ID NOs: 1, 54, 200 or 211.

21. The method according to claim 20, wherein the sequence of the first region of the oligomer is at least 80% identical to the sequence of a region of at least 10 contiguous monomers present in SEQ ID NO: 169 or 180.

22. The method according to claim 17, wherein the protein tyrosine kinase inhibitor is selected from the group consisting of gefitinib, imatinib, erlotinib, canertinib, vandetanib, lapatinib, sorafenib, AG-494, RG-13022, RG-14620, BIBW 2992, tyrphostin AG-825, tyrphostin 9, tyrphostin 23, tyrphostin 25, tyrphostin 46, tyrphostin 47, tyrphostin 53, butein, curcumin, AG-1478, AG-879, cyclopropanecarboxylic acid-(3-(6-(3-trifluoromethyl-phenylamino)-pyrimidin-4-ylamino)-phenyl)-amide, N8-(3-Chloro-4-fluorophenyl)-N2-(1-methylpiperidin-4-yl)-pyrimido[5,4-d]pyrimidine-2,8-diamine, 2HCl (CAS 196612-93-8), 4-(4-benzyloxyanilino)-6,7-dimethoxyquinazoline, N-(4-((3-Chloro-4-fluorophenyl)amino)pyrido[3,4-d]pyrimidin-6-yl)2-butynamide (CAS 881001-19-0), EKB-569, HKI-272, and HKI-357.

23. The method according to claim 17, wherein the at least one monomer in the first region of the oligomer is a nucleoside analog selected from the group consisting of an LNA monomer, a monomer containing a 2′-O-alkyl-ribose sugar, a monomer containing a 2′-O-methyl-ribose sugar, a monomer containing a 2′-amino-deoxyribose sugar, and a monomer containing a 2′fluoro-deoxyribose sugar.

24. The method according to claim 23, wherein the at least one monomer in the first region of the oligomer is an LNA monomer.

25. The method of claim 17, wherein the mammal was previously treated with a protein tyrosine kinase inhibitor.

26. The method of claim 17, wherein the mammal was not previously treated with a protein tyrosine kinase inhibitor.

27. The method of claim 17, wherein the protein tyrosine kinase inhibitor is gefitinib.

28. A method of treating cancer in a mammal, comprising administering to said mammal an effective amount of an oligomer consisting of the sequence:
(SEQ ID NO: 180)
5′-T_{S}A_{S}G_{S}c_{S}c_{S}t_{S}g_{S}t_{S}c_{S}a_{S}c_{S}t_{S}t_{S}^{Me}C_{S}T_{S}^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base, and
wherein the cancer is resistant to treatment with a protein tyrosine kinase inhibitor.

29. The method of claim 28, wherein the protein tyrosine kinase inhibitor is gefitinib.

30. (canceled)

31. The method according to claim 17, wherein said conjugate is a conjugate of an oligomer consisting of the sequence:
(SEQ ID NO: 180)
5′-T_{S}A_{S}G_{S}c_{S}c_{S}t_{S}g_{S}t_{S}c_{S}a_{S}c_{S}t_{S}t_{S}^{Me}C_{S}T_{S}^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base.

32. A method of inhibiting the proliferation of a mammalian cancer cell comprising contacting said cell with an effective amount of an oligomer consisting of 10 to 50 contiguous monomers wherein adjacent monomers are covalently linked by a phosphate group or a phosphorothioate group,
wherein said oligomer comprises a first region of at least 10 contiguous monomers;
wherein at least one monomer of said first region is a nucleoside analog;
wherein the sequence of said first region is at least 80% identical to the reverse complement of the best-aligned target region of a mammalian HER3 gene or a mammalian HER3 mRNA; and
wherein proliferation of the mammalian cancer cell is not inhibited by a protein tyrosine kinase inhibitor.

33. The method of claim 32, wherein said oligomer consists of the sequence:
(SEQ ID NO: 180)
5′-T_{S}A_{S}G_{S}c_{S}c_{S}t_{S}g_{S}t_{S}c_{S}a_{S}c_{S}t_{S}t_{S}^{Me}C_{S}T_{S}^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base.

34. The method of claim 33, wherein the proliferation of said cell is inhibited by at least 50% when compared to the proliferation of an untreated cell of the same type.

35. The method of claim 32, wherein the mammalian cancer cell is a non-small cell lung cancer cell.


SEQUENCE LISTING (249 sequences)

SEQ ID NO: 1 - DNA, 16 bp, Artificial Sequence
  Description of Artificial Sequence Synthetic oligonucleotide
gctccagacatcactc

SEQ ID NO: 2 - DNA, 15 bp, Artificial Sequence
  Description of Artificial Sequence Synthetic oligonucleotide
gctccagacatcact

SEQ ID NO: 3 - DNA, 15 bp, Artificial Sequence
  Description of Artificial Sequence Synthetic oligonucleotide
ctccagacatcactc

SEQ ID NO: 4 - DNA, 14 bp, Artificial Sequence
  Description of Artificial Sequence Synthetic oligonucleotide
gctccagacatcac

SEQ ID NO: 5 - DNA, 14 bp, Artificial Sequence
  Description of Artificial Sequence Synthetic oligonucleotide
ctccagacatcact

SEQ ID NO: 6 - DNA, 14 bp, Artificial Sequence
  Description of Artificial Sequence Synthetic oligonucleotide
tccagacatcactc

SEQ ID NO: 7 - DNA, 13 bp, Artificial Sequence
  Description of Artificial Sequence Synthetic oligonucleotide
gctccagacatca

SEQ ID NO: 8 - DNA, 13 bp, Artificial Sequence
  Description of Artificial Sequence Synthetic oligonucleotide
ctccagacatcac

SEQ ID NO: 9 - DNA, 13 bp, Artificial Sequence
  Description of Artificial Sequence Synthetic oligonucleotide
tccagacatcact

SEQ ID NO: 10 - DNA, 13 bp, Artificial Sequence
  Description of Artificial Sequence Synthetic oligonucleotide
ccagacatcactc

SEQ ID NO: 11 - DNA, 12 bp, Artificial Sequence
  Description of Artificial Sequence Synthetic oligonucleotide
gctccagacatc

SEQ ID NO: 12 - DNA, 12 bp, Artificial Sequence
  Description of Artificial Sequence Synthetic oligonucleotide
ctccagacatca

SEQ ID NO: 13 - DNA, 12 bp, Artificial Sequence
  Description of Artificial Sequence Synthetic oligonucleotide
tccagacatcac

SEQ ID NO: 14 - DNA, 12 bp, Artificial Sequence
  Description of Artificial Sequence Synthetic oligonucleotide
ccagacatcact

SEQ ID NO: 15 - DNA, 12 bp, Artificial Sequence
  Description of Artificial Sequence Synthetic oligonucleotide
cagacatcactc

SEQ ID NO: 16 - DNA, 16 bp, Artificial Sequence
  Description of Artificial Sequence Synthetic oligonucleotide
ctccagacatcactct

SEQ ID NO: 17 - DNA, 16 bp, Artificial Sequence
  Description of Artificial Sequence Synthetic oligonucleotide
cagacatcactctggt

SEQ ID NO: 18 - DNA, 16 bp, Artificial Sequence
  Description of Artificial Sequence Synthetic oligonucleotide
agacatcactctggtg

SEQ ID NO: 19 - DNA, 16 bp, Artificial Sequence
  Description of Artificial Sequence Synthetic oligonucleotide
atagctccagacatca

SEQ ID NO: 20 - DNA, 15 bp, Artificial Sequence
  Description of Artificial Sequence Synthetic oligonucleotide
atagctccagacatc

SEQ ID NO: 21 - DNA, 15 bp, Artificial Sequence
  Description of Artificial Sequence Synthetic oligonucleotide
tagctccagacatca

SEQ ID NO: 22 - DNA, 14 bp, Artificial Sequence
  Description of Artificial Sequence Synthetic oligonucleotide
atagctccagacat

SEQ ID NO: 23 - DNA, 14 bp, Artificial Sequence
  Description of Artificial Sequence Synthetic oligonucleotide
tagctccagacatc

SEQ ID NO: 24 - DNA, 14 bp, Artificial Sequence
  Description of Artificial Sequence Synthetic oligonucleotide
agctccagacatca

SEQ ID NO: 25 - DNA, 13 bp, Artificial Sequence
  Description of Artificial Sequence Synthetic oligonucleotide
atagctccagaca

SEQ ID NO: 26 - DNA, 13 bp, Artificial Sequence
  Description of Artificial Sequence Synthetic oligonucleotide
tagctccagacat

SEQ ID NO: 27 - DNA, 13 bp, Artificial Sequence
  Description of Artificial Sequence Synthetic oligonucleotide
agctccagacatc

SEQ ID NO: 28 - DNA, 13 bp, Artificial Sequence
  Description of Artificial Sequence Synthetic oligonucleotide
gctccagacatca

SEQ ID NO: 29 - DNA, 12 bp, Artificial Sequence
  Description of Artificial Sequence Synthetic oligonucleotide
atagctccagac

SEQ ID NO: 30 - DNA, 12 bp, Artificial Sequence
  Description of Artificial Sequence Synthetic oligonucleotide
tagctccagaca

SEQ ID NO: 31 - DNA, 12 bp, Artificial Sequence
  Description of Artificial Sequence Synthetic oligonucleotide
agctccagacat

SEQ ID NO: 32 - DNA, 12 bp, Artificial Sequence
  Description of Artificial Sequence Synthetic oligonucleotide
gctccagacatc

SEQ ID NO: 33 - DNA, 12 bp, Artificial Sequence
  Description of Artificial Sequence Synthetic oligonucleotide
ctccagacatca

SEQ ID NO: 34 - DNA, 16 bp, Artificial Sequence
  Description of Artificial Sequence Synthetic oligonucleotide
tcacaccatagctcca

SEQ ID NO: 35 - DNA, 15 bp, Artificial Sequence
  Description of Artificial Sequence Synthetic oligonucleotide
tcacaccatagctcc

SEQ ID NO: 36 - DNA, 15 bp, Artificial Sequence
  Description of Artificial Sequence Synthetic oligonucleotide
cacaccatagctcca

SEQ ID NO: 37 - DNA, 14 bp, Artificial Sequence
  Description of Artificial Sequence Synthetic oligonucleotide
tcacaccatagctc

SEQ ID NO: 38 - DNA, 14 bp, Artificial Sequence
  Description of Artificial Sequence Synthetic oligonucleotide
cacaccatagctcc

SEQ ID NO: 39 - DNA, 14 bp, Artificial Sequence
  Description of Artificial Sequence Synthetic oligonucleotide
acaccatagctcca

SEQ ID NO: 40 - DNA, 13 bp, Artificial Sequence
  Description of Artificial Sequence Synthetic oligonucleotide
tcacaccatagct

SEQ ID NO: 41 - DNA, 13 bp, Artificial Sequence
  Description of Artificial Sequence Synthetic oligonucleotide
cacaccatagctc

SEQ ID NO: 42 - DNA, 13 bp, Artificial Sequence
  Description of Artificial Sequence Synthetic oligonucleotide
acaccatagctcc

SEQ ID NO: 43 - DNA, 13 bp, Artificial Sequence
  Description of Artificial Sequence Synthetic oligonucleotide
caccatagctcca

SEQ ID NO: 44 - DNA, 12 bp, Artificial Sequence
  Description of Artificial Sequence Synthetic oligonucleotide
tcacaccatagc

SEQ ID NO: 45 - DNA, 12 bp, Artificial Sequence
  Description of Artificial Sequence Synthetic oligonucleotide
cacaccatagct

SEQ ID NO: 46 - DNA, 12 bp, Artificial Sequence
  Description of Artificial Sequence Synthetic oligonucleotide
acaccatagctc

SEQ ID NO: 47 - DNA, 12 bp, Artificial Sequence
  Description of Artificial Sequence Synthetic oligonucleotide
caccatagctcc

SEQ ID NO: 48 - DNA, 12 bp, Artificial Sequence
  Description of Artificial Sequence Synthetic oligonucleotide
accatagctcca

SEQ ID NO: 49 - DNA, 16 bp, Artificial Sequence
  Description of Artificial Sequence Synthetic oligonucleotide
catccaacacttgacc

SEQ ID NO: 50 - DNA, 16 bp, Artificial Sequence
  Description of Artificial Sequence Synthetic oligonucleotide
atccaacacttgacca

SEQ ID NO: 51 - DNA, 16 bp, Artificial Sequence
  Description of Artificial Sequence Synthetic oligonucleotide
caatcatccaacactt

SEQ ID NO: 52 - DNA, 16 bp, Artificial Sequence
  Description of Artificial Sequence Synthetic oligonucleotide
tcaatcatccaacact

SEQ ID NO: 53 - DNA, 16 bp, Artificial Sequence
  Description of Artificial Sequence Synthetic oligonucleotide
catgtagacatcaatt

SEQ ID NO: 54 - DNA, 16 bp, Artificial Sequence
  Description of Artificial Sequence Synthetic oligonucleotide
tagcctgtcacttctc

SEQ ID NO: 55 - DNA, 16 bp, Artificial Sequence
  Description of Artificial Sequence Synthetic oligonucleotide
agatggcaaacttccc

SEQ ID NO: 56 - DNA, 16 bp, Artificial Sequence
  Description of Artificial Sequence Synthetic oligonucleotide
caaggctcacacatct

SEQ ID NO: 57 - DNA, 16 bp, Artificial Sequence
  Description of Artificial Sequence Synthetic oligonucleotide
aagtccaggttgccca

SEQ ID NO: 58 - DNA, 16 bp, Artificial Sequence
  Description of Artificial Sequence Synthetic oligonucleotide
cattcaagttcttcat

SEQ ID NO: 59 - DNA, 16 bp, Artificial Sequence
  Description of Artificial Sequence Synthetic oligonucleotide
cactaatttccttcag

SEQ ID NO: 60 - DNA, 15 bp, Artificial Sequence
  Description of Artificial Sequence Synthetic oligonucleotide
cactaatttccttca

SEQ ID NO: 61 - DNA, 15 bp, Artificial Sequence
  Description of Artificial Sequence Synthetic oligonucleotide
actaatttccttcag

SEQ ID NO: 62 - DNA, 14 bp, Artificial Sequence
  Description of Artificial Sequence Synthetic oligonucleotide
cactaatttccttc

SEQ ID NO: 63 - DNA, 14 bp, Artificial Sequence
  Description of Artificial Sequence Synthetic oligonucleotide
actaatttccttca

SEQ ID NO: 64 - DNA, 14 bp, Artificial Sequence
  Description of Artificial Sequence Synthetic oligonucleotide
ctaatttccttcag

SEQ ID NO: 65 - DNA, 13 bp, Artificial Sequence
  Description of Artificial Sequence Synthetic oligonucleotide
cactaatttcctt

SEQ ID NO: 66 - DNA, 13 bp, Artificial Sequence
  Description of Artificial Sequence Synthetic oligonucleotide
actaatttccttc

SEQ ID NO: 67 - DNA, 13 bp, Artificial Sequence
  Description of Artificial Sequence Synthetic oligonucleotide
ctaatttccttca

SEQ ID NO: 68 - DNA, 13 bp, Artificial Sequence
  Description of Artificial Sequence Synthetic oligonucleotide
taatttccttcag

SEQ ID NO: 69 - DNA, 12 bp, Artificial Sequence
  Description of Artificial Sequence Synthetic oligonucleotide
cactaatttcct

SEQ ID NO: 70 - DNA, 12 bp, Artificial Sequence
  Description of Artificial Sequence Synthetic oligonucleotide
actaatttcctt

SEQ ID NO: 71 - DNA, 12 bp, Artificial Sequence
  Description of Artificial Sequence Synthetic oligonucleotide
ctaatttccttc

SEQ ID NO: 72 - DNA, 12 bp, Artificial Sequence
  Description of Artificial Sequence Synthetic oligonucleotide
taatttccttca

SEQ ID NO: 73 - DNA, 12 bp, Artificial Sequence
  Description of Artificial Sequence Synthetic oligonucleotide
aatttccttcag

SEQ ID NO: 74 - DNA, 16 bp, Artificial Sequence
  Description of Artificial Sequence Synthetic oligonucleotide
gcccagcactaatttc

SEQ ID NO: 75 - DNA, 16 bp, Artificial Sequence
  Description of Artificial Sequence Synthetic oligonucleotide
ctttgccctctgccac

SEQ ID NO: 76 - DNA, 16 bp, Artificial Sequence
  Description of Artificial Sequence Synthetic oligonucleotide
cacacactttgccctc

SEQ ID NO: 77 - DNA, 15 bp, Artificial Sequence
  Description of Artificial Sequence Synthetic oligonucleotide
cacacactttgccct

SEQ ID NO: 78 - DNA, 15 bp, Artificial Sequence
  Description of Artificial Sequence Synthetic oligonucleotide
acacactttgccctc

SEQ ID NO: 79 - DNA, 14 bp, Artificial Sequence
  Description of Artificial Sequence Synthetic oligonucleotide
cacacactttgccc

SEQ ID NO: 80 - DNA, 14 bp, Artificial Sequence
  Description of Artificial Sequence Synthetic oligonucleotide
acacactttgccct

SEQ ID NO: 81 - DNA, 14 bp, Artificial Sequence
  Description of Artificial Sequence Synthetic oligonucleotide
cacactttgccctc

SEQ ID NO: 82 - DNA, 13 bp, Artificial Sequence
  Description of Artificial Sequence Synthetic oligonucleotide
cacacactttgcc

SEQ ID NO: 83 - DNA, 13 bp, Artificial Sequence
  Description of Artificial Sequence Synthetic oligonucleotide
acacactttgccc

SEQ ID NO: 84 - DNA, 13 bp, Artificial Sequence
  Description of Artificial Sequence Synthetic oligonucleotide
cacactttgccct

SEQ ID NO: 85 - DNA, 13 bp, Artificial Sequence
  Description of Artificial Sequence Synthetic oligonucleotide
acactttgccctc

SEQ ID NO: 86 - DNA, 12 bp, Artificial Sequence
  Description of Artificial Sequence Synthetic oligonucleotide
cacacactttgc

SEQ ID NO: 87 - DNA, 12 bp, Artificial Sequence
  Description of Artificial Sequence Synthetic oligonucleotide
acacactttgcc

SEQ ID NO: 88 - DNA, 12 bp, Artificial Sequence
  Description of Artificial Sequence Synthetic oligonucleotide
cacactttgccc

SEQ ID NO: 89 - DNA, 12 bp, Artificial Sequence
  Description of Artificial Sequence Synthetic oligonucleotide
acactttgccct

SEQ ID NO: 90 - DNA, 12 bp, Artificial Sequence
  Description of Artificial Sequence Synthetic oligonucleotide
cactttgccctc

SEQ ID NO: 91 - DNA, 16 bp, Artificial Sequence
  Description of Artificial Sequence Synthetic oligonucleotide
cagttccaaagacacc

SEQ ID NO: 92 - DNA, 16 bp, Artificial Sequence
  Description of Artificial Sequence Synthetic oligonucleotide
tggcaatttgtactcc

SEQ ID NO: 93 - DNA, 15 bp, Artificial Sequence
  Description of Artificial Sequence Synthetic oligonucleotide
tggcaatttgtactc

SEQ ID NO: 94 - DNA, 15 bp, Artificial Sequence
  Description of Artificial Sequence Synthetic oligonucleotide
ggcaatttgtactcc

SEQ ID NO: 95 - DNA, 14 bp, Artificial Sequence
  Description of Artificial Sequence Synthetic oligonucleotide
tggcaatttgtact

SEQ ID NO: 96 - DNA, 14 bp, Artificial Sequence
  Description of Artificial Sequence Synthetic oligonucleotide
ggcaatttgtactc

SEQ ID NO: 97 - DNA, 14 bp, Artificial Sequence
  Description of Artificial Sequence Synthetic oligonucleotide
gcaatttgtactcc

SEQ ID NO: 98 - DNA, 13 bp, Artificial Sequence
  Description of Artificial Sequence Synthetic oligonucleotide
tggcaatttgtac

SEQ ID NO: 99 - DNA, 13 bp, Artificial Sequence
  Description of Artificial Sequence Synthetic oligonucleotide
ggcaatttgtact

SEQ ID NO: 100 - DNA, 13 bp, Artificial Sequence
  Description of Artificial Sequence Synthetic oligonucleotide
gcaatttgtactc

SEQ ID NO: 101 - DNA, 13 bp, Artificial Sequence
  Description of Artificial Sequence Synthetic oligonucleotide
caatttgtactcc

SEQ ID NO: 102 - DNA, 12 bp, Artificial Sequence
  Description of Artificial Sequence Synthetic oligonucleotide
tggcaatttgta

SEQ ID NO: 103 - DNA, 12 bp, Artificial Sequence
  Description of Artificial Sequence Synthetic oligonucleotide
ggcaatttgtac

SEQ ID NO: 104 - DNA, 12 bp, Artificial Sequence
  Description of Artificial Sequence Synthetic oligonucleotide
gcaatttgtact

SEQ ID NO: 105 - DNA, 12 bp, Artificial Sequence
  Description of Artificial Sequence Synthetic oligonucleotide
caatttgtactc

SEQ ID NO: 106 - DNA, 12 bp, Artificial Sequence
  Description of Artificial Sequence Synthetic oligonucleotide
aatttgtactcc

SEQ ID NO: 107 - DNA, 16 bp, Artificial Sequence
  Description of Artificial Sequence Synthetic oligonucleotide
gtgtgtgtatttccca

SEQ ID NO: 108 - DNA, 15 bp, Artificial Sequence
  Description of Artificial Sequence Synthetic oligonucleotide
gtgtgtgtatttccc

SEQ ID NO: 109 - DNA, 15 bp, Artificial Sequence
  Description of Artificial Sequence Synthetic oligonucleotide
tgtgtgtatttccca

SEQ ID NO: 110 - DNA, 14 bp, Artificial Sequence
  Description of Artificial Sequence Synthetic oligonucleotide
gtgtgtgtatttcc

SEQ ID NO: 111 - DNA, 14 bp, Artificial Sequence
  Description of Artificial Sequence Synthetic oligonucleotide
tgtgtgtatttccc

SEQ ID NO: 112 - DNA, 14 bp, Artificial Sequence
  Description of Artificial Sequence Synthetic oligonucleotide
gtgtgtatttccca

SEQ ID NO: 113 - DNA, 13 bp, Artificial Sequence
  Description of Artificial Sequence Synthetic oligonucleotide
gtgtgtgtatttc

SEQ ID NO: 114 - DNA, 13 bp, Artificial Sequence
  Description of Artificial Sequence Synthetic oligonucleotide
tgtgtgtatttcc

SEQ ID NO: 115 - DNA, 13 bp, Artificial Sequence
  Description of Artificial Sequence Synthetic oligonucleotide
gtgtgtatttccc

SEQ ID NO: 116 - DNA, 13 bp, Artificial Sequence
  Description of Artificial Sequence Synthetic oligonucleotide
tgtgtatttccca

SEQ ID NO: 117 - DNA, 12 bp, Artificial Sequence
  Description of Artificial Sequence Synthetic oligonucleotide
gtgtgtgtattt

SEQ ID NO: 118 - DNA, 12 bp, Artificial Sequence
  Description of Artificial Sequence Synthetic oligonucleotide
tgtgtgtatttc

SEQ ID NO: 119 - DNA, 12 bp, Artificial Sequence
  Description of Artificial Sequence Synthetic oligonucleotide
gtgtgtatttcc

SEQ ID NO: 120 - DNA, 12 bp, Artificial Sequence
  Description of Artificial Sequence Synthetic oligonucleotide
tgtgtatttccc

SEQ ID NO: 121 - DNA, 12 bp, Artificial Sequence
  Description of Artificial Sequence Synthetic oligonucleotide
gtgtatttccca

SEQ ID NO: 122 - DNA, 16 bp, Artificial Sequence
  Description of Artificial Sequence Synthetic oligonucleotide
ccctctgatgactctg

SEQ ID NO: 123 - DNA, 15 bp, Artificial Sequence
  Description of Artificial Sequence Synthetic oligonucleotide
ccctctgatgactct

SEQ ID NO: 124 - DNA, 15 bp, Artificial Sequence
  Description of Artificial Sequence Synthetic oligonucleotide
cctctgatgactctg

SEQ ID NO: 125 - DNA, 14 bp, Artificial Sequence
  Description of Artificial Sequence Synthetic oligonucleotide
ccctctgatgactc

SEQ ID NO: 126 - DNA, 14 bp, Artificial Sequence
  Description of Artificial Sequence Synthetic oligonucleotide
cctctgatgactct

SEQ ID NO: 127 - DNA, 14 bp, Artificial Sequence
  Description of Artificial Sequence Synthetic oligonucleotide
ctctgatgactctg

SEQ ID NO: 128 - DNA, 13 bp, Artificial Sequence
  Description of Artificial Sequence Synthetic oligonucleotide
ccctctgatgact

SEQ ID NO: 129 - DNA, 13 bp, Artificial Sequence
  Description of Artificial Sequence Synthetic oligonucleotide
cctctgatgactc

SEQ ID NO: 130 - DNA, 13 bp, Artificial Sequence
  Description of Artificial Sequence Synthetic oligonucleotide
ctctgatgactct

SEQ ID NO: 131 - DNA, 13 bp, Artificial Sequence
  Description of Artificial Sequence Synthetic oligonucleotide
tctgatgactctg

SEQ ID NO: 132 - DNA, 12 bp, Artificial Sequence
  Description of Artificial Sequence Synthetic oligonucleotide
ccctctgatgac

SEQ ID NO: 133 - DNA, 12 bp, Artificial Sequence
  Description of Artificial Sequence Synthetic oligonucleotide
cctctgatgact

SEQ ID NO: 134 - DNA, 12 bp, Artificial Sequence
  Description of Artificial Sequence Synthetic oligonucleotide
ctctgatgactc

SEQ ID NO: 135 - DNA, 12 bp, Artificial Sequence
  Description of Artificial Sequence Synthetic oligonucleotide
tctgatgactct

SEQ ID NO: 136 - DNA, 12 bp, Artificial Sequence
  Description of Artificial Sequence Synthetic oligonucleotide
ctgatgactctg

SEQ ID NO: 137 - DNA, 16 bp, Artificial Sequence
  Description of Artificial Sequence Synthetic oligonucleotide
catactcctcatcttc

SEQ ID NO: 138 - DNA, 16 bp, Artificial Sequence
  Description of Artificial Sequence Synthetic oligonucleotide
ccaccacaaagttatg

SEQ ID NO: 139 - DNA, 16 bp, Artificial Sequence
  Description of Artificial Sequence Synthetic oligonucleotide
catcactctggtgtgt

SEQ ID NO: 140 - DNA, 16 bp, Artificial Sequence
  Description of Artificial Sequence Synthetic oligonucleotide
gacatcactctggtgt

SEQ ID NO: 141 - DNA, 16 bp, Artificial Sequence
  Description of Artificial Sequence Synthetic oligonucleotide
gctccagacatcactc

SEQ ID NO: 142 - DNA, 16 bp, Artificial Sequence
  Description of Artificial Sequence Synthetic oligonucleotide
ctccagacatcactct

SEQ ID NO: 143 - DNA, 16 bp, Artificial Sequence
  Description of Artificial Sequence Synthetic oligonucleotide
cagacatcactctggt

SEQ ID NO: 144 - DNA, 16 bp, Artificial Sequence
  Description of Artificial Sequence Synthetic oligonucleotide
agacatcactctggtg

SEQ ID NO: 145 - DNA, 16 bp, Artificial Sequence
  Description of Artificial Sequence Synthetic oligonucleotide
atagctccagacatca

SEQ ID NO: 146 - DNA, 16 bp, Artificial Sequence
  Description of Artificial Sequence Synthetic oligonucleotide
tcacaccatagctcca

SEQ ID NO: 147 - DNA, 16 bp, Artificial Sequence
  Description of Artificial Sequence Synthetic oligonucleotide
catccaacacttgacc

SEQ ID NO: 148 - DNA, 16 bp, Artificial Sequence
  Description of Artificial Sequence Synthetic oligonucleotide
atccaacacttgacca

SEQ ID NO: 149 - DNA, 16 bp, Artificial Sequence
  Description of Artificial Sequence Synthetic oligonucleotide
caatcatccaacactt

SEQ ID NO: 150 - DNA, 16 bp, Artificial Sequence
  Description of Artificial Sequence Synthetic oligonucleotide
tcaatcatccaacact

SEQ ID NO: 151 - DNA, 16 bp, Artificial Sequence
  Description of Artificial Sequence Synthetic oligonucleotide
catgtagacatcaatt

SEQ ID NO: 152 - DNA, 16 bp, Artificial Sequence
  Description of Artificial Sequence Synthetic oligonucleotide
tagcctgtcacttctc

SEQ ID NO: 153 - DNA, 16 bp, Artificial Sequence
  Description of Artificial Sequence Synthetic oligonucleotide
agatggcaaacttccc

SEQ ID NO: 154 - DNA, 16 bp, Artificial Sequence
  Description of Artificial Sequence Synthetic oligonucleotide
caaggctcacacatct

SEQ ID NO: 155 - DNA, 16 bp, Artificial Sequence
  Description of Artificial Sequence Synthetic oligonucleotide
aagtccaggttgccca

SEQ ID NO: 156 - DNA, 16 bp, Artificial Sequence
  Description of Artificial Sequence Synthetic oligonucleotide
cattcaagttcttcat

SEQ ID NO: 157 - DNA, 16 bp, Artificial Sequence
  Description of Artificial Sequence Synthetic oligonucleotide
cactaatttccttcag

SEQ ID NO: 158 - DNA, 16 bp, Artificial Sequence
  Description of Artificial Sequence Synthetic oligonucleotide
gcccagcactaatttc

SEQ ID NO: 159 - DNA, 16 bp, Artificial Sequence
  Description of Artificial Sequence Synthetic oligonucleotide
ctttgccctctgccac

SEQ ID NO: 160 - DNA, 16 bp, Artificial Sequence
  Description of Artificial Sequence Synthetic oligonucleotide
cacacactttgccctc

SEQ ID NO: 161 - DNA, 16 bp, Artificial Sequence
  Description of Artificial Sequence Synthetic oligonucleotide
cagttccaaagacacc

SEQ ID NO: 162 - DNA, 16 bp, Artificial Sequence
  Description of Artificial Sequence Synthetic oligonucleotide
tggcaatttgtactcc

SEQ ID NO: 163 - DNA, 16 bp, Artificial Sequence
  Description of Artificial Sequence Synthetic oligonucleotide
gtgtgtgtatttccca

SEQ ID NO: 164 - DNA, 16 bp, Artificial Sequence
  Description of Artificial Sequence Synthetic oligonucleotide
ccctctgatgactctg

SEQ ID NO: 165 - DNA, 16 bp, Artificial Sequence
  Description of Artificial Sequence Synthetic oligonucleotide
catactcctcatcttc

SEQ ID NO: 166 - DNA, 16 bp, Artificial Sequence
  Description of Artificial Sequence Synthetic oligonucleotide
ccaccacaaagttatg

SEQ ID NO: 167 - DNA, 16 bp, Artificial Sequence
  Description of Artificial Sequence Synthetic oligonucleotide
catcactctggtgtgt

SEQ ID NO: 168 - DNA, 16 bp, Artificial Sequence
  Description of Artificial Sequence Synthetic oligonucleotide
gacatcactctggtgt

SEQ ID NO: 169 - DNA, 16 bp, Artificial Sequence
  Description of Artificial Sequence Synthetic oligonucleotide
gctccagacatcactc

SEQ ID NO: 170 - DNA, 16 bp, Artificial Sequence
  Description of Artificial Sequence Synthetic oligonucleotide
ctccagacatcactct

SEQ ID NO: 171 - DNA, 16 bp, Artificial Sequence
  Description of Artificial Sequence Synthetic oligonucleotide
cagacatcactctggt

SEQ ID NO: 172 - DNA, 16 bp, Artificial Sequence
  Description of Artificial Sequence Synthetic oligonucleotide
agacatcactctggtg

SEQ ID NO: 173 - DNA, 16 bp, Artificial Sequence
  Description of Artificial Sequence Synthetic oligonucleotide
atagctccagacatca

SEQ ID NO: 174 - DNA, 16 bp, Artificial Sequence
  Description of Artificial Sequence Synthetic oligonucleotide
tcacaccatagctcca

SEQ ID NO: 175 - DNA, 16 bp, Artificial Sequence
  Description of Artificial Sequence Synthetic oligonucleotide
catccaacacttgacc

SEQ ID NO: 176 - DNA, 16 bp, Artificial Sequence
  Description of Artificial Sequence Synthetic oligonucleotide
atccaacacttgacca

SEQ ID NO: 177 - DNA, 16 bp, Artificial Sequence
  Description of Artificial Sequence Synthetic oligonucleotide
caatcatccaacactt

SEQ ID NO: 178 - DNA, 16 bp, Artificial Sequence
  Description of Artificial Sequence Synthetic oligonucleotide
tcaatcatccaacact

SEQ ID NO: 179 - DNA, 16 bp, Artificial Sequence
  Description of Artificial Sequence Synthetic oligonucleotide
catgtagacatcaatt

SEQ ID NO: 180 - DNA, 16 bp, Artificial Sequence
  Description of Artificial Sequence Synthetic oligonucleotide
tagcctgtcacttctc

SEQ ID NO: 181 - DNA, 16 bp, Artificial Sequence
  Description of Artificial Sequence Synthetic oligonucleotide
agatggcaaacttccc

SEQ ID NO: 182 - DNA, 16 bp, Artificial Sequence
  Description of Artificial Sequence Synthetic oligonucleotide
caaggctcacacatct

SEQ ID NO: 183 - DNA, 16 bp, Artificial Sequence
  Description of Artificial Sequence Synthetic oligonucleotide
aagtccaggttgccca

SEQ ID NO: 184 - DNA, 16 bp, Artificial Sequence
  Description of Artificial Sequence Synthetic oligonucleotide
cattcaagttcttcat

SEQ ID NO: 185 - DNA, 16 bp, Artificial Sequence
  Description of Artificial Sequence Synthetic oligonucleotide
cactaatttccttcag

SEQ ID NO: 186 - DNA, 16 bp, Artificial Sequence
  Description of Artificial Sequence Synthetic oligonucleotide
gcccagcactaatttc

SEQ ID NO: 187 - DNA, 16 bp, Artificial Sequence
  Description of Artificial Sequence Synthetic oligonucleotide
ctttgccctctgccac

SEQ ID NO: 188 - DNA, 16 bp, Artificial Sequence
  Description of Artificial Sequence Synthetic oligonucleotide
cacacactttgccctc

SEQ ID NO: 189 - DNA, 16 bp, Artificial Sequence
  Description of Artificial Sequence Synthetic oligonucleotide
cagttccaaagacacc

SEQ ID NO: 190 - DNA, 16 bp, Artificial Sequence
  Description of Artificial Sequence Synthetic oligonucleotide
tggcaatttgtactcc

SEQ ID NO: 191 - DNA, 16 bp, Artificial Sequence
  Description of Artificial Sequence Synthetic oligonucleotide
gtgtgtgtatttccca

SEQ ID NO: 192 - DNA, 16 bp, Artificial Sequence
  Description of Artificial Sequence Synthetic oligonucleotide
ccctctgatgactctg

SEQ ID NO: 193 - DNA, 16 bp, Artificial Sequence
  Description of Artificial Sequence Synthetic oligonucleotide
catactcctcatcttc

SEQ ID NO: 194 - DNA, 16 bp, Artificial Sequence
  Description of Artificial Sequence Synthetic oligonucleotide
ccaccacaaagttatg

SEQ ID NO: 195 - DNA, 16 bp, Artificial Sequence
  Description of Artificial Sequence Synthetic oligonucleotide
catcactctggtgtgt

SEQ ID NO: 196 - DNA, 16 bp, Artificial Sequence
  Description of Artificial Sequence Synthetic oligonucleotide
gacatcactctggtgt

SEQ ID NO: 197 - DNA, 5511 bp, homo sapiens
acacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatcca

SEQ ID NO: 198 - DNA, 5616 bp, homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 199 - DNA, 4624 bp, homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 200 - DNA, 24 bp, Artificial Sequence
  Description of Artificial Sequence Synthetic oligonucleotide
catagctccagacatcactctggt

SEQ ID NO: 201 - DNA, 24 bp, Artificial Sequence
  Description of Artificial Sequence Synthetic oligonucleotide
atagctccagacatcactctggtg

SEQ ID NO: 202 - DNA, 24 bp, Artificial Sequence
  Description of Artificial Sequence Synthetic oligonucleotide
gctccagacatcactctggtgtgt

SEQ ID NO: 203 - DNA, 24 bp, Artificial Sequence
  Description of Artificial Sequence Synthetic oligonucleotide
ctccagacatcactctggtgtgtg

SEQ ID NO: 204 - DNA, 24 bp, Artificial Sequence
  Description of Artificial Sequence Synthetic oligonucleotide
caccatagctccagacatcactct

SEQ ID NO: 205 - DNA, 24 bp, Artificial Sequence
  Description of Artificial Sequence Synthetic oligonucleotide
actgtcacaccatagctccagaca

SEQ ID NO: 206 - DNA, 24 bp, Artificial Sequence
  Description of Artificial Sequence Synthetic oligonucleotide
caatcatccaacacttgaccatca

SEQ ID NO: 207 - DNA, 24 bp, Artificial Sequence
  Description of Artificial Sequence Synthetic oligonucleotide
aatcatccaacacttgaccatcac

SEQ ID NO: 208 - DNA, 24 bp, Artificial Sequence
  Description of Artificial Sequence Synthetic oligonucleotide
tcatcaatcatccaacacttgacc

SEQ ID NO: 209 - DNA, 24 bp, Artificial Sequence
  Description of Artificial Sequence Synthetic oligonucleotide
ctcatcaatcatccaacacttgac

SEQ ID NO: 210 - DNA, 24 bp, Artificial Sequence
  Description of Artificial Sequence Synthetic oligonucleotide
tcaccatgtagacatcaattgtgc

SEQ ID NO: 211 - DNA, 24 bp, Artificial Sequence
  Description of Artificial Sequence Synthetic oligonucleotide
gacatagcctgtcacttctcgaat

SEQ ID NO: 212 - DNA, 24 bp, Artificial Sequence
  Description of Artificial Sequence Synthetic oligonucleotide
acgaagatggcaaacttcccatcg

SEQ ID NO: 213 - DNA, 24 bp, Artificial Sequence
  Description of Artificial Sequence Synthetic oligonucleotide
cccacaaggctcacacatcttgag

SEQ ID NO: 214 - DNA, 24 bp, Artificial Sequence
  Description of Artificial Sequence Synthetic oligonucleotide
cagaaagtccaggttgcccaggat

SEQ ID NO: 215 - DNA, 24 bp, Artificial Sequence
  Description of Artificial Sequence Synthetic oligonucleotide
gtgacattcaagttcttcatgatc

SEQ ID NO: 216 - DNA, 24 bp, Artificial Sequence
  Description of Artificial Sequence Synthetic oligonucleotide
ccagcactaatttccttcagggat

SEQ ID NO: 217 - DNA, 24 bp, Artificial Sequence
  Description of Artificial Sequence Synthetic oligonucleotide
atacgcccagcactaatttccttc

SEQ ID NO: 218 - DNA, 24 bp, Artificial Sequence
  Description of Artificial Sequence Synthetic oligonucleotide
cacactttgccctctgccacgcag

SEQ ID NO: 219 - DNA, 24 bp, Artificial Sequence
  Description of Artificial Sequence Synthetic oligonucleotide
gggtcacacactttgccctctgcc

SEQ ID NO: 220 - DNA, 24 bp, Artificial Sequence
  Description of Artificial Sequence Synthetic oligonucleotide
tgcacagttccaaagacacccgag

SEQ ID NO: 221 - DNA, 24 bp, Artificial Sequence
  Description of Artificial Sequence Synthetic oligonucleotide
cccttggcaatttgtactccccag

SEQ ID NO: 222 - DNA, 24 bp, Artificial Sequence
  Description of Artificial Sequence Synthetic oligonucleotide
tctggtgtgtgtatttcccaaagt

SEQ ID NO: 223 - DNA, 24 bp, Artificial Sequence
  Description of Artificial Sequence Synthetic oligonucleotide
atgcccctctgatgactctgatgc

SEQ ID NO: 224 - DNA, 24 bp, Artificial Sequence
  Description of Artificial Sequence Synthetic oligonucleotide
tattcatactcctcatcttcatct

SEQ ID NO: 225 - DNA, 24 bp, Artificial Sequence
  Description of Artificial Sequence Synthetic oligonucleotide
tgatccaccacaaagttatgggga

SEQ ID NO: 226 - DNA, 24 bp, Artificial Sequence
  Description of Artificial Sequence Synthetic oligonucleotide
cagacatcactctggtgtgtgtat

SEQ ID NO: 227 - DNA, 24 bp, Artificial Sequence
  Description of Artificial Sequence Synthetic oligonucleotide
tccagacatcactctggtgtgtgt

SEQ ID NO: 228 - DNA, 15 bp, Artificial Sequence
  Description of Artificial Sequence Synthetic oligonucleotide
tagcctgtcacttct

SEQ ID NO: 229 - DNA, 15 bp, Artificial Sequence
  Description of Artificial Sequence Synthetic oligonucleotide
agcctgtcacttctc

SEQ ID NO: 230 - DNA, 14 bp, Artificial Sequence
  Description of Artificial Sequence Synthetic oligonucleotide
tagcctgtcacttc

SEQ ID NO: 231 - DNA, 14 bp, Artificial Sequence
  Description of Artificial Sequence Synthetic oligonucleotide
agcctgtcacttct

SEQ ID NO: 232 - DNA, 13 bp, Artificial Sequence
  Description of Artificial Sequence Synthetic oligonucleotide
tagcctgtcactt

SEQ ID NO: 233 - DNA, 12 bp, Artificial Sequence
  Description of Artificial Sequence Synthetic oligonucleotide
tagcctgtcact

SEQ ID NO: 234 - DNA, 13 bp, Artificial Sequence
  Description of Artificial Sequence Synthetic oligonucleotide
tagcctgtcactt

SEQ ID NO: 235 - DNA, 16 bp, Artificial
  LNA oligomers
cgtcagtatgcgaatc

SEQ ID NO: 236 - DNA, 16 bp, Artificial
  LNA oligomers
cgcagattagaaacct

SEQ ID NO: 237 - DNA, 22 bp, Artificial
  probe sequence
ccacacctggtcatagcggtga

SEQ ID NO: 238 - DNA, 20 bp, Artificial
  probe sequence
ctgtttaggccaagcagagg

SEQ ID NO: 239 - DNA, 20 bp, Artificial
  probe sequence
attctgaatcctgcgtccac

SEQ ID NO: 240 - DNA, 20 bp, Artificial
  probe sequence
cattgcccaacctccgcgtg

SEQ ID NO: 241 - DNA, 20 bp, Artificial
  probe sequence
tgcagtggattcgagaagtg

SEQ ID NO: 242 - DNA, 20 bp, Artificial
  probe sequence
ggcaaacttcccatcgtaga

SEQ ID NO: 243 - DNA, 20 bp, Artificial
  probe sequence
actggcgctgccaaggctgt

SEQ ID NO: 244 - DNA, 20 bp, Artificial
  probe sequence
ccacccagaagactgtggat

SEQ ID NO: 245 - DNA, 20 bp, Artificial
  probe sequence
ttcagctcagggatgacctt

SEQ ID NO: 246 - DNA, 20 bp, Artificial
  probe sequence
agctgtggcgtgatggccgt

SEQ ID NO: 247 - DNA, 20 bp, Artificial
  probe sequence
aactttggcattgtggaagg

SEQ ID NO: 248 - DNA, 20 bp, Artificial
  probe sequence
ggatgcagggatgatgttct

SEQ ID NO: 249 - DNA, 16 bp, Artificial
  LNA oligomers
tagcctttgacctctc